(19) United States
(12) Patent Application Publication
(10) Pub. No.: US 2006/0277632 A1
(43) Pub. Date: Dec. 7, 2006

(54) Methods for production of chitin and chitosan

(75) Inventors: Brian Carr, Raleigh, NC (US); Philip E. Hammer, Cary, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Athenix Corporation, Durham, NC (US)

(21) Appl. No.: 11/434,526

(22) Filed: May 15, 2006

Related U.S. Application Data
(60) Provisional application No. 60/680,942, filed on May 13, 2005.

(51) Int. Cl. A01H 1/00; C08B 37/08; C07H 21/04; C12P 19/28; C12N 1/21; C12N 1/16; C12N 15/74

(52) U.S. Cl. 800/284; 435/85; 435/252.33; 435/254.3; 435/484; 435/488; 536/20; 536/23.2

(57) ABSTRACT

Compositions and methods for producing chitin and chitosan are provided. The compositions comprise genetically modified organisms, including fungi, yeast, bacterial and plant organisms that have been engineered to express heterologous genes involved in chitin and chitosan synthesis. Microorganisms and plants that have been modified for production of chitin and/or chitosan within the vacuole of a cell are encompassed. Methods for production of chitin also comprise culturing the genetically engineered organisms in conditions that allow for chitin production. Further methods include converting the chitin to chitosan by a chemical process. Production of chitosan also comprises culturing organisms that are genetically modified to produce chitosan without the need for chemical modification. Methods for in vitro chitosan production are also encompassed.

### CROSS REFERENCE TO RELATED APPLICATION

This application claims the benefit of U.S. Provisional Application Ser. No. 60/680,942, filed May 13, 2005, the contents of which are herein incorporated by reference in its entirety.

### FIELD OF THE INVENTION

This invention relates to the field of molecular biology. Provided are compositions and methods for producing chitin and/or chitosan.

### BACKGROUND OF THE INVENTION

Chitin is a natural polysaccharide present in various marine and terrestrial organisms, including crustacea, insects, mollusks, and microorganism or plants, such as fungi. Chitin is typically an amorphous solid that is largely insoluble in water, dilute acids, and alkali. Although chitin has various commercial applications, greater commercial utility is found by converting chitin to the deacetylated product chitosan.

Chitosan can be created by N-deacetylation of the chitin polymer. It is an amorphous solid that is largely insoluble in water, but is soluble in aqueous organic acids, such as formic and acetic acids. Chitosan has many industrial, medical, pharmaceutical, and nutritional uses, including those requiring a biodegradable, non-toxic polymer. For example, chitosan is used as a polyelectrolytic coagulant and a sludge dewatering aid in wastewater treatment. Medical, pharmaceutical, and nutritional uses often require a higher quality chitosan for functional and aesthetic reasons. These uses include applications as anticoagulants, antiviral agents, drug carriers, cosmetic additives, dialysis membranes, orthopedic materials, wound dressings, food stabilizers and thickeners, flavor and nutrient carriers, and dietary fiber.

Commercially produced chitosan is currently harvested from shellfish by a lengthy extraction process in which chitin is chemically deacetylated to chitosan using strong alkali treatment. The resulting chitosan is then isolated as an acid soluble material. Though chitosan has numerous industrial uses, the requirement for raw material and the lengthy extraction process contribute to high production costs that limit actual industrial use of this polymer. In fact, the potential industrial use of chitosan exceeds the production capacity of many traditional production schemes. Given the industrial high demand for chitosan, methods aimed at improving its production are needed.

Chitin and chitosan are related polymers that are produced by several types of fungi and yeasts as part of their cell wall. One approach to improving commercial chitosan production methods would be to engineer existing fermentation strains to produce more chitin/chitosan as a value-added product. For example, Rhizopus oryzae or Aspergillus niger strains utilized for citric acid production contain substantial amounts of chitin/chitosan, and thus are an attractive source for a value-added approach. Alternatively, a fermentative yeast can be engineered to produce chitosan as a component of its cell walls. Regardless of the approach, when chitin/chitosan are to be produced as a value-added product in an existing fermentation system, care must be taken to ensure that the yield of the primary fermentation product is not reduced and that the processing of the primary fermentation product is not altered in the process. An attractive approach would be to develop a cell expression system that is dedicated to production of chitin/chitosan.

Numerous publications report use of fungal biomass for production and recovery of chitosan. Methods of chitosan production from microbial biomass, such as filamentous fungi, were disclosed in U.S. Pat. No. 4,806,474, International application No. WO 01/68714, and other publications (Synowiecki and Al-Khateeb (2003) Crit Rev Food Sci Nutr 43(2): 145-171; Pochanavanich and Suntomsuk (2002) Lett Appl Microbiol 35(1):17-21). However, these processes yield relatively expensive chitosan as compared to product extracted from shellfish. The yield of extracted chitosan is limited by the chitin and chitosan contents in the biomass.

Other methods of producing chitosan involve recovery from microbial biomass, such as the methods taught by U.S. Pat. No. 4,806,474 and U.S. Patent Application No. 20050042735, herein incorporated by reference. Another method, taught by U.S. Pat. No. 4,282,351, teaches only how to create a chitosan-beta-glucan complex.

Methods for reducing the cost of chitosan production are needed in order to realize the industrial potential for this polymer.

### BRIEF SUMMARY OF THE INVENTION

Compositions and methods for producing chitin and chitosan are provided. The compositions comprise genetically modified organisms, including fungi, yeast, and bacterial organisms that have been mutated or engineered to express heterologous genes involved in chitin and chitosan synthesis. Genes including chitin synthase, glutamine-fructose-6-phosphate aminotransferase (GFA), and chitin deacetylase can be mutated, for example, to allow production of chitin as an insoluble polymer within a cell (including within vacuole compartments), to have improved processivity, increased reaction velocity, a modified Kₘ for a substrate, a substrate preference for UDP-glucosamine, or to allow for secretion of chitin from the cell. Alternatively, heterologous genes from viral, fungal, insect, or other organisms may be expressed in the organism of choice to produce increased amounts of chitin, or to produce chitosan directly, without the need for chemical modification of chitin.

Compositions also include polynucleotides encoding enzymes or polypeptides involved in the production of chitin and/or chitosan (“chitin/chitosan-related sequences”), vectors comprising those polynucleotides, and host cells comprising the vectors. Compositions comprising a coding sequence for one or more polypeptides involved in the production of chitin and/or chitosan are provided. Compositions of the present invention further include synthetic polynucleotides encoding enzymes or polypeptides involved in the production of chitin and/or chitosan. The coding sequences can be used in DNA constructs or expression cassettes for transformation and expression in organisms, including microorganisms and plants. Compositions also comprise transformed fungi, bacteria, plants, plant cells, tissues, and seeds. In addition, methods are provided for producing the polypeptides encoded by the synthetic nucleotides of the invention.

In particular, isolated polynucleotides corresponding to chitin/chitosan-related sequences are provided. Additionally, amino acid sequences corresponding to the polynucleotides are encompassed. In particular, the present invention provides for isolated polynucleotides comprising variants or fragments of the the polynucleotide sequence set forth in SEQ ID NOS:1, 3, 5, 7, 9, 11, 13, 19, 21, 23, 25, 27, 29, 31, 33, 35, 37, 39, 41, and 43 or polynucleotide sequences encoding variants and fragments of the amino acid sequence shown in SEQ ID NOS:2, 4, 6, 8, 10, 12, 14, 15, 16, 17, 18, 20, 22, 24, 26, 28, 30, 32, 34, 36, 38, 40, 42 and 44. Nucleotide sequences that are complementary to a nucleotide sequence of the invention, or that hybridize to a sequence of the invention are also encompassed.

Methods for production of chitin comprise culturing the genetically engineered organisms in conditions that allow for chitin production, and isolating the chitin. Methods for production of chitosan comprise culturing the genetically engineered organisms in conditions that allow for chitin production, isolating the chitin, and converting the chitin to chitosan by a chemical process. Production of chitosan can also comprise culturing organisms that are genetically modified to produce chitosan without the need for chemical modification. Methods for in vitro chitosan production comprise expressing chitin synthase in a first organism, isolating and purifying the synthase, expressing chitin deacetylase in a second organism, isolating and purifying the deacetylase, linking the synthase and deacetylase to a resin, contacting the resin with a permeabilized cell that expresses GFA, and isolating the chitosan. Additional embodiment include generating a modified synthase enzyme that utilizes UDP-N-acetylglucosamine as well as UDP-glucosamine-1-phosphate as substrate, culturing a cell expressing the modified synthase enzyme in the presence of UDP-N-acetylglucosamine and UDP-glucosamine-1-phosphate, and isolating the chitin and/or chitosan produced by the cell.

### BRIEF DESCRIPTION OF THE DRAWING

FIG. 1 shows the enzymatic process of chitin and chitosan production from UDP-N-acetyl-glucosamine.

### DETAILED DESCRIPTION OF THE INVENTION

I. Overview

The present invention is directed to compositions and methods for chitin and chitosan production. The compositions are fingi, yeast, bacteria, and plants that have been genetically engineered to enhance production of particular enzyme substrates in the chitin/chitosan pathway and to increase the rate of enzyme reactions such that increased amounts of chitin and/or chitosan are produced. Methods of improving chitin and/or chitosan production comprise culturing these genetically engineered organisms under conditions suitable for expression of the chitin and/or chitosan and isolating the desired polymer. The chitin can be converted to chitosan by methods known in the art, for example, by treatment with strong alkali, or can be converted to chitosan directly in the cell by modified enzymes. These modified enzymes can include chitin deacetylase enzymes that have been modifed to improve expression or activity or both, and/or can include a chitin synthase that has been modifed to catalyze the polymerization of both chitin and/or chitosan.

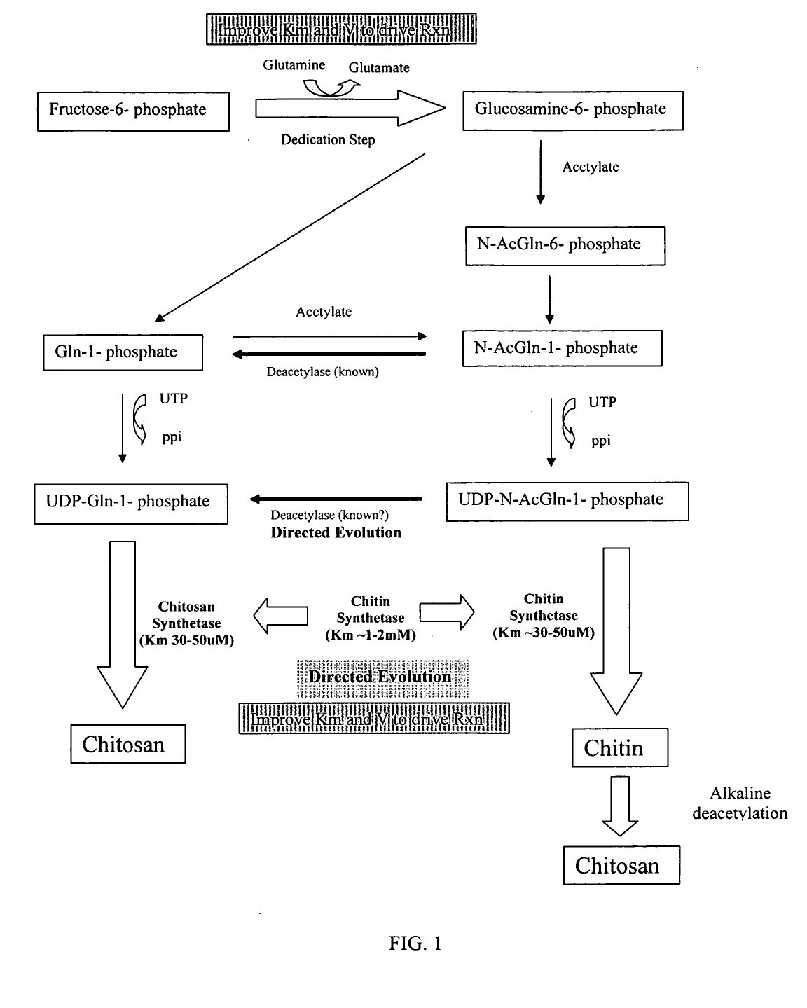

Chitin is produced by the polymerization of UDP-N-acetyl-glucosamine (referred to herein as “UDP-NAc-Gln”) by the enzyme chitin synthase (see FIG. 1). Chitin synthases (also referred to as “chitin synthases”) are large, membrane-associated enzymes that polymerize UDP-NAc-Gln into linear chains of chitin (NAc-Gln, see FIG. 1).

The first dedicated step in chitin and chitosan pathway in fungi is the isomerization and amination of fructose-6-P to form glucosamine-6-P, a reaction catalyzed by glutamine-fructose-6-P amidotransferase (Gfalp; Enzyme Commission No. 2.6.1.16) encoded by GFA1. Glucosamine-6-P is N-acetylated to form N-acetylglucosamine-6-P by glucosamine-6-P acetyltransferase (EC 2.3.1.4) encoded by GNA1. Phospho-N-acetylglucosamine mutase (EC 5.4.2.3), encoded by AGMI converts N-acetylglucosamine-6-P to N-acetylglucosamine-1-P, which is further converted to UDP-N-acetylglucosamine by UDP-N-acetylglucosaminepyrophosphorylase (EC 2.7.7.23), encoded by UAP1. The enzyme chitin synthase (EC 2.4.1.16) catalyzes polymerization of N-acetylglucosaminyl units by using UDP-N-acetylglucosamine as substrate. The reaction takes place on the plasma membrane. The enzyme utilizes UDP-N-acetylglucosamine present in the cytoplasm. The elongated polymer chains are extruded through the plasmalemma to the cell exterior. An UDP-N-acetylglucosamine transporter encoded by YEA4 is localized in the endoplasmic reticulum and it appears to be important to chitin synthesis. Chitin deacetylases (EC 3.5.1.41), encoded by CDA1 and CDA2, convert nascent chitin to chitosan by hydrolyzing the acetyl group from amino sugar units; the enzyme is inactive with preformed chitin as substrate.

In this pathway, chitin synthase is a key enzyme for many reasons. First, as the synthase, chitin synthase has a large affect on the chain length, rate of synthesis, and composition of the polymers formed. For example, the processivity of the enzyme (i.e., the average chain length produced) is an interplay between the affinity of the enzyme for its substrate, the rate of the reaction, and the average residence time on any one polymer. Thus, by utilizing a synthase with a high processivity, one can increase the chain length of chitin formed. Chitin synthase also controls the sugar composition of the polymer formed. In nature, chitin synthases incorporate only NAc-Gln into polymers. However, alteration of the specificity of the synthase to incorporate glucosamine at a higher frequency opens the possibility for de novo synthesis of chitosan.

Chitin synthases have been isolated from many organisms, including Aspergillus, Rhizopus, yeast, shellfish, and insects. Many fungi have multiple chitin synthases, only a subset of which appear to be critical in the production of large amounts of cell wall chitin. The number of chitin synthase isoenzymes varies from one copy in the yeast, S. pombe, to seven copies in some filamentous fungi, such as A. fumigatus. The yeast, S. cerevisiae, contains three chitin synthases: Chs1p (SEQ ID NO:2) encoded by CHS1 (SEQ ID NO: 1), Chs2p (SEQ ID NO:4) encoded by CHSII (SEQ ID NO:3) and Chs3p (SEQ ID NO:6) encoded by CHSIII (SEQ ID NO:5). These synthases differ with respect to the peptide sequences, temporal and spatial expression patterns, and enzyme characteristics such as optimum pH, metal specificity, and susceptibility to inhibitors. Chs3p is responsible for the synthesis of 90 to 95% of the cellular chitin in yeast. Its optimal activity requires the involvement of four other regulatory proteins, encoded by CHS4 to CHS7, in its translocation and localization. Yeast strains defective in any of these genes have drastically reduced chitin synthesis.

Studies of UDP-NAc-Gln synthesis as early as the 1950's suggest that formation of glucosamine-6 phosphate from fructose-6-phosphate (by glutamine-fructose 6-phosphate aminotransferase, “GFA”) is a key commitment step in the synthesis of amino sugars. Genetic evidence in yeast, as well studies of a chitin-producing virus (CVK2), provide direct support for the importance of GFA in regulating the flow of precursors to amino sugar biosynthesis. Another potentially key enzyme regulating chitin production is the UDP transferase that primes NAc-Gln for incorporation into polymer.

Ultimately the extent of polymer formation is an interplay between the rate of polymer synthesis, the availability of precursor(s), and the rate of degradation of a polymer. Thus, for chitin synthesis, the activity and processivity of the chitin synthase, as well as the rate of formation of UDP-NAc-Gln are critical to achieving high chitin/chitosan levels. To achieve substantial chitosan, not only is a high rate of chitin synthesis required, but chitin deacetylase must be of sufficient activity that it can deacetylate a substantial portion of nascent chains.

Chitosan and chitin are often found in fungal and yeast cell walls. Chitosan is produced by the action of chitin deacetylase (see FIG. 1). During polymerization of chitin by chitin synthase, the activity of a chitin deacetylase (if present) can lead to accumulation of deacetylated monomers within the growing chain. Sufficient deacetylase activity yields molecules with a substantial percentage of deacetylated monomers, yielding chitosan. Experiments with chitin deacetylase have shown that this enzyme does not deacetylate monomeric UDP-NAc-Gln, and has little activity on fully formed chitin. Rather the deacetylase acts on the growing chitin chain.

Genes and their products involved in the metabolic pathways leading to chitin and chitosan formation have been characterized in some microorganism or plants (Farkas (1979) Microbiol Rev 43(2):117-144). Chitin synthesis in the yeast cell wall has been investigated most extensively. FIG. 1 illustrates the general metabolic pathway for chitin and chitosan synthesis. The nomenclature of different enzymes and their alternative names can be found at the enzyme site of the ExPASy Molecular Biology Servers of the Swiss Institute of Bioinformatics. The nucleotide sequences of the identified or cloned genes and the amino acid sequences of their expression products are available in the databases at the National Center for Biotechnology Information and or in the ExPASy database.

It is known in the art that the enzymes having the same biological activity may have different names depending on from what organism the enzyme is derived. The following is a general listing and discussion of alternate names for many of the enzymes referenced herein and specific names of genes encoding such enzymes from some organisms. The enzyme names can be used interchangeably, or as appropriate for a given sequence or organism, although the invention intends to encompass enzymes of a given function from any organism. Therefore, for example, while glucosamine-fructose-6-phosphate aminotransferase is the name typically used to refer to an enzyme in yeast and other fungi, general reference to “a glucosamine-fructose-6-phosphate aminotransferase” will be intended to refer to structural/functional homologues of the yeast enzyme from other types of microorganism or plants, plants and animals that are known in the art or to structural/functional homologues that are synthetically produced or produced by classical mutagenesis. For example, in bacteria, glucosamine-fructose-6-phosphate aminotransferase is commonly called glucosamine-6-phosphate synthase or glucosamine-6-phosphate synthetase. However, a general reference herein to glucosamine-fructose-6-phosphate aminotransferase without specifically identifying the source can include a bacterial glucosamine-6-phosphate synthase.

For example, the enzyme generally referred to herein as “glucosamine-6-phosphate synthase” catalyzes the formation of glucosamine-6-phosphate and glutamate from fructose-6-phosphate and glutamine. The enzyme is also known as glucosamine-fructose-6-phosphate aminotransferase (isomerizing); hexosephosphate aminotransferase; D-fructose-6-phosphate amidotransferase; glucosamine-6-phosphate isomerase (glutamine-forming); L-glutamine-fructose-6-phosphate amidotransferase; and GlcN6P synthase. The glucosamine-6-phosphate synthase from E. coli and other bacteria is generally referred to as GlmS. The glucosamine-6-phosphate synthase from yeast and other sources is generally referred to as GFA1 or GFAT.

Glucosamine-fructose-6-phosphate aminotransferases from a variety of organisms are known in the art and are contemplated for use in the genetic engineering strategies of the present invention. For example, the glucosamine-fructose-6-phosphate aminotransferase (GFA1) from Saccharomyces cerevisiae is described herein, and which has an amino acid sequence represented herein by SEQ ID NO:8, encoded by a polynucleotide sequence represented herein by SEQ ID NO:7. The glucosamine-fructose-6-phosphate aminotransferase from Escherichia coli is also described herein, which in bacteria is termed glucosamine-6-phosphate synthase. The glucosamine-6-phosphate synthase from E. coli has an amino acid sequence represented herein by SEQ ID NO:10, which is encoded by a polynucleotide sequence represented herein by SEQ ID NO:9. Also described herein is the glucosamine-6-phosphate synthase from Bacillus subtilis, which has an amino acid sequence represented herein by SEQ ID NO:12, encoded by a polynucleotide sequence represented herein by SEQ ID NO:11. Glucosamine-fructose-6-phosphate aminotransferases (GFA1) from other microorganism or plants are also known in the art, such as from Candida albicans, which has an amino acid sequence represented herein by SEQ ID NO:14, encoded by a polynucleotide sequence represented herein by SEQ ID NO:13. Also included in the invention are glucosamine-fructose-6-phosphate aminotransferases which have one or more genetic modifications that lead to an increase in the production of chitin and/or chitosan. In general, according to the present invention, an increase or a decrease in a given characteristic of a mutant or modified enzyme is made with reference to the same characteristic of a wild-type (i.e., normal, not modified) enzyme from the same organism which is measured or established under the same or equivalent conditions (discussed in more detail below). An “increase in the production of chitin and/or chitosan” can refer to an increase in the synthesis of chitin and/or chitosan, can refer to an increase in the deacetylation of chitin to form chitosan, or can refer to both an increase in the synthesis of chitin and/or chitosan and an increase in the deacetylation of chitin to form chitosan. A genetic modification that leads to or results in an increase in the production of chiton and/or chitosan includes any genetic modification that causes any detectable or measurable change or modification in the chitin and/or chitosan biosynthetic pathway expressed by the organism as compared to in the absence of the genetic modification. A detectable change or modification in the chitin and/or chitosan biosynthetic pathway can include, but is not limited to, a detectable change in the production of at least one product in the chitin and/or chitosan biosynthetic pathway, or a detectable change in the production of chitin and/or chitosan by the microorganism or plant in which it is expressed. A detectable change can include an increase in chitin and/or chitosan production of about 10%, about 20%, about 30%, 40%, 50%, 75%, 100%, 150%, 200%, 250%, 300%, about 400% or greater compared to production in an organism that has not been modified.

The enzyme generally referred to herein as chitin synthase catalyzes the polymerization of N-acetylglucosamine using UDP-N-acetylglucosamine as donor. Chitin synthase can also be referred to as chitin-UDP acetyl-glucosaminyl transferase. Chitin synthase from a variety of organisms are known in the art and are contemplated for use in the genetic engineering strategies of the present invention. Numerous forms of chitin synthase enzymes and their nucleotide sequences have been identified in many different organisms, especially yeast and fungi. The amino acid and nucleotide sequences can be found in the NCBI and ExPASy databases. These include, but are not limited to, Saccharomyces cerevisiae CHS1 (SEQ ID NOS:2 and 1; GENEBANK® Accession Nos. P08004 and M14045, respectively), CHS3 (SEQ ID NO:6; Accession No. P29465), CHS4 (also known as SKT5, SEQ ID NO:15; Accession No. NP_{—}009492), CHS5 (SEQ ID NO:16; Accession No. NP_{—}013434), CHS6 (SEQ ID NO:17; Accession No. NP_{—}012436), and CHS7 (SEQ ID NO:18; Accession No. NP_{—}012011); Aspergillus niger CHS1-ASPNG (SEQ ID NO:19 and 20; Accession No. P30581) and CHS2-ASPNG (SEQ ID NO:21 and 22; Accession No. P30582); A. fumigatus CHSC_ASPFU (SEQ ID NOS:24 and 23; Accession Nos. Q92197 and X94245, respectively), CHSD_ASPFU (SEQ ID NO:25 and 26; Accession No. P78746), and CHSG_ASPFU (SEQ ID NO:27 and 28; Accession No. P54267); and Aspergillus orzae chitin synthase (SEQ ID NOS:30 and 29; Accession Nos. AAK31732.1 and AY029261, respectively), chsZ (SEQ ID NO:31 and 32; Accession No. AB081655), and chsY (SEQ ID NO:33 and 34; Accession No. AB081655). Also included in the invention are chitin synthases that have a genetic modification that, when expressed in a cell, results in an increase in the production of chitin and/or chitosan.

The enzyme generally referred to herein as chitin deacetylase hydrolyses the N-acetyl group from amino sugar units of the nascent chitin to form chitosan (EC. 3.5.1.41). Chitin deacetylases from a variety of organisms are known in the art and are contemplated for use in the genetic engineering strategies of the present invention. For example, two chitin deacetylases from Saccharomyces cerevisiae are described herein. The chitin deacetylase from S. cerevisiae known as CDA1 has an amino acid sequence represented herein by SEQ ID NO:36, which is encoded by a polynucleotide sequence represented herein by SEQ ID NO:35. The chitin deacetylase from S. cerevisiae known as CDA2 has an amino acid sequence represented herein by SEQ ID NO:38, which is encoded by a polynucleotide sequence represented herein by SEQ ID NO:37. The fungal chitin deacetylase amino acid and nucleotide sequence from Mucor rouxii are represented by SEQ ID NOS:40 and 39, respectively (Accession No. Z19109). The fungal chitin deacetylase amino acid and nucleotide sequences from Gongronella butleri are represented by SEQ ID NOS:42 and 41, respectively (Accession Nos. AAN65362 and AF411810). Also included in the invention are chitin deacetylases that have a genetic modification that, when expressed in a cell, results in an increase in the production of chitin and/or chitosan.

To the extent that genes, other polynucleotide sequences, and amino acid sequences from a particular microorganism or plant are discussed and/or exemplified below, it will be appreciated that other microorganism or plants have similar metabolic pathways, as well as genes and proteins having similar structure and function within such pathways. As such, the principles discussed below with regard to any particular microorganism or plant, either as a source of genetic material or a host cell to be modified, are applicable to other microorganism or plants and are expressly encompassed by the present invention.

II. Compositions

A. Genetically Modified Organisms

In general, a microorganism or plant having a genetically modified (also referred to as genetically engineered) metabolic pathway for the production of chitin and/or chitosan has at least one genetic modification, as discussed in detail below, which results in a change in one or more genes, enzymatic reactions, or pathways as described above as compared to a wild-type microorganism or plant cultured under the same conditions. Such a modification in a microorganism or plant changes the ability of the microorganism or plant to produce chitin and/or chitosan. As discussed in detail below, according to the present invention, a genetically modified microorganism or plant preferably has an enhanced ability to produce chitin and/or chitosan as compared to a wild-type microorganism or plant of the same species (and preferably the same strain), which is cultured under the same or equivalent conditions. Equivalent conditions are culture conditions which are similar, but not necessarily identical (e.g., some changes in medium composition, temperature, pH and other conditions can be tolerated), and which do not substantially change the effect on microbe growth or production of chitin or chitosan by the microbe.

In general, according to the present invention, an increase or a decrease in a given characteristic of a mutant or modified enzyme (e.g., enzyme activity) is made with reference to the same characteristic of a wild-type (i.e., normal, not modified) enzyme that is derived from the same organism (from the same source or parent sequence), which is measured or established under the same or equivalent conditions. Similarly, an increase or decrease in a characteristic of a genetically modified microorganism or plant (e.g., expression and/or biological activity of a protein, or production of a product) is made with reference to the same characteristic of a wild-type microorganism or plant of the same species, and preferably the same strain, under the same or equivalent conditions. Such conditions include the assay or culture conditions (e.g., medium components, temperature, pH, etc.) under which the activity of the protein (e.g., expression or biological activity) or other characteristic of the microorganism or plant is measured, as well as the type of assay used, the host microorganism or plant that is evaluated, etc. As discussed above, equivalent conditions are conditions (e.g., culture conditions) which are similar, but not necessarily identical (e.g., some conservative changes in conditions can be tolerated), and which do not substantially change the effect on microbe growth, enzyme expression or biological activity as compared to a comparison made under the same conditions.

Preferably, a genetically modified microorganism or plant that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism or plant, of at least about 5%, at least about 10%, at least about 15%, 20%, 25%, 30%, 35%, 40%, 45%, 50%, 55%, 60%, 65%, 70%, 75%, 80%, 85%, 90%, 95%, 96%, 97%, 98%, 99%, 100% or greater. The same differences are preferred when comparing an isolated modified polynucleotide or protein directly to the isolated wild-type polynucleotide or protein (e.g., if the comparison is done in vitro as compared to in vivo).

In another aspect of the invention, a genetically modified microorganism or plant that has a genetic modification that increases or decreases the activity of a given protein (e.g., an enzyme) has an increase or decrease, respectively, in the activity (e.g., expression, production and/or biological activity) of the protein, as compared to the activity of the wild-type protein in a wild-type microorganism or plant, of at least about 2-fold, at least about 5-fold, 10-fold, 20-fold, 30-fold, 40-fold, 50-fold, 75-fold, 100-fold, 125-fold, 150-fold, or greater.

For genetic engineering to increase chitin and chitosan content, suitable fungal hosts include, but are not limited to, Ascomycetes, Zygomycetes and Deuteromycetes. Suitable genera include, but are not limited to, Aspergillus, Absidia, Gongronella, Lentinus, Mucor, Phycomyces, Rhizopus, Chrysosporium, Neurospora and Trichoderma. Suitable fungal species include, but are not limited to, Aspergillus niger, Aspergillus terrreus, A. nidulans, Aspergillus orzae, Absidia coerulea, Absidia repens, Absidia blakesleeana, Gongronella butleri, Lentinus endodes, Mucor rouxii, Phycomyces blakesleenaus, Rhizopus oryzae, Chrysosporium lucknowense, Neurospora crassa, N. intermedia and Trichoderm reesei.

For genetic engineering to increase chitin and chitosan content, suitable genera of yeast include, but are not limited to, Saccharomyces, Schizosaccharomyces, Candida, Hansenula, Pichia, Kluyveromyces, and Phaffia. Suitable yeast species include, but are not limited to, Saccharomyces cerevisiae, Schizosaccharomyces pombe, Candida albicans, Candida guillermondii, Hansenula polymorpha, Pichia pastoris, P. canadensis, Kluyveromyces marxianus and Phaffia rhodozyma.

The present invention may also be used for producing chitin and/or chitosan in any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

This invention is particularly suitable for any member of the monocot plant family including, but not limited to, maize, rice, barley, oats, wheat, sorghum, rye, sugarcane, pineapple, yams, onion, banana, coconut, and dates.

B. Isolated Polynucleotides, and Variants and Fragments Thereof

One aspect of the invention pertains to isolated polynucleotides comprising nucleotide sequences encoding chitin/chitosan-related proteins and polypeptides or biologically active portions thereof, as well as polynucleotides sufficient for use as hybridization probes to identify chitin/chitosan-related polynucleotides. As used herein, the term “polynucleotide” is intended to include DNA molecules (e.g., cDNA or genomic DNA) and RNA molecules (e.g., mRNA) and analogs of the DNA or RNA generated using nucleotide analogs. The polynucleotides can be single-stranded or double-stranded, but preferably are double-stranded DNA.

Nucleotide sequences encoding the proteins of the present invention include the variants and fragments of the sequences set forth in SEQ ID NOS:1, 3, 5, 7, 9, 11, 13, 19, 21, 23, 25, 27, 29, 31, 33, 35, 37, 39, 41, and 43, wherein the variant or fragment is a chitin/chitosan-related polynucleotide that encodes a polypeptide that has been modified to increase the production of chitin and/or chitosan in a host cell or suitable reaction media. By “complement” is intended a polynucleotide sequence that is sufficiently complementary to a given nucleotide sequence such that it can hybridize to the given nucleotide sequence to thereby form a stable duplex. The invention also encompasses polynucleotides comprising nucleotide sequences encoding partial-length chitin/chitosan-related proteins, and complements thereof.

An “isolated” or “purified” polynucleotide or protein, or biologically active portion thereof, is substantially free of other cellular material, or culture medium when produced by recombinant techniques, or substantially free of chemical precursors or other chemicals when chemically synthesized. Preferably, an “isolated” polynucleotide is free of sequences (preferably protein encoding sequences) that naturally flank the polynucleotide (i.e., sequences located at the 5′ and 3′ ends of the polynucleotide) in the genomic DNA of the organism from which the polynucleotide is derived. For purposes of the invention, “isolated” when used to refer to polynucleotides excludes isolated chromosomes. For example, in various embodiments, the isolated chitin/chitosan-related polynucleotide can contain less than about 5 kb, 4 kb, 3 kb, 2 kb, 1 kb, 0.5 kb, or 0.1 kb of nucleotide sequence that naturally flanks the polynucleotide in genomic DNA of the cell from which the polynucleotide is derived. A chitin/chitosan-related protein that is substantially free of cellular material includes preparations of protein having less than about 30%, 20%, 10%, or 5% (by dry weight) of non-chitin/chitosan-related protein (also referred to herein as a “contaminating protein”).

Polynucleotides that are fragments of these chitin/chitosan-related nucleotide sequences are also encompassed by the present invention. By “fragment” is intended a portion of a nucleotide sequence encoding a chitin/chitosan-related protein. A fragment of a nucleotide sequence may encode a biologically active portion of a chitin/chitosan-related protein, or it may be a fragment that can be used as a hybridization probe or PCR primer using methods disclosed below. Polynucleotides that are fragments of a chitin/chitosan-related nucleotide sequence comprise at least about 15, 20, 50, 75, 100, 200, 300, 350, 400, 450, 500, 550, 600, 650, 700, 750, 800, 850, 900, 950, 1000, 1050, 1100, 1150, 1200, 1250, 1300, 1350 contiguous nucleotides, or up to the number of nucleotides present in a full-length chitin/chitosan-related nucleotide sequence disclosed herein. By “contiguous” nucleotides is intended nucleotide residues that are immediately adjacent to one another.

Fragments of the nucleotide sequences of the present invention generally will encode protein fragments that retain the biological activity of the full-length chitin/chitosan-related protein; i.e., activity associated with the production of chitin and/or chitosan. By “retains chitin/chitosan-related activity” is intended that the fragment will have at least about 30%, at least about 50%, at least about 70%, or at least about 80% or greater of the chitin/chitosan-related activity of the full-length chitin/chitosan-related protein disclosed herein as SEQ ID NOS:2, 4, 6, 8, 10, 12, 14, 15, 16, 17, 18, 20, 22, 24, 26, 28, 30, 32, 34, 36, 38, 40, 42 or 44. The activity of enzymes and polypeptides involved in the production of chitin and/or chitosan can be measured using standard assays, or can be measured based on the production of chitin and/or chitosan. Methods for measuring chitin and/or chitosan are described in Lehmann and White (1975) Infection and Immunity 12(5):987-992.

A fragment of a chitin/chitosan-related nucleotide sequence that encodes a biologically active portion of a protein of the invention will encode at least about 15, 25, 30, 50, 75, 100, 125, 150, 175, 200, 250, 300, 350, 400 contiguous amino acids, or up to the total number of amino acids present in a full-length chitin/chitosan-related protein of the invention.

Preferred chitin/chitosan-related proteins of the present invention are encoded by a nucleotide sequence sufficiently identical to the nucleotide sequence of SEQ ID NOS: 1, 3, 5, 7, 9, 11, 13, 19, 21, 23, 25, 27, 29, 31, 33, 35, 37, 39, 41, or 43. The term “sufficiently identical” is intended an amino acid or nucleotide sequence that has at least about 60% or 65% sequence identity, about 70% or 75% sequence identity, about 80% or 85% sequence identity, or about 90%, 91%, 92%, 93%, 94%, 95%, 96%, 97%, 98% or 99% sequence identity compared to a reference sequence using one of the alignment programs described herein using standard parameters. One of skill in the art will recognize that these values can be appropriately adjusted to determine corresponding identity of proteins encoded by two nucleotide sequences by taking into account codon degeneracy, amino acid similarity, reading frame positioning, and the like.

To determine the percent identity of two amino acid sequences or of two polynucleotides, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. BLAST nucleotide searches can be performed with the BLASTN program, score=100, wordlength=12, to obtain nucleotide sequences homologous to polynucleotides of the invention. BLAST protein searches can be performed with the BLASTX program, score=50, wordlength=3, to obtain amino acid sequences homologous to chitin/chitosan-related protein molecules of the invention. To obtain gapped alignments for comparison purposes, Gapped BLAST can be utilized as described in Altschul et al. (1997) Polynucleotides Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. See www.ncbi.nlm.nih.gov. Another non-limiting example of a mathematical algorithm utilized for the comparison of sequences is the ClustalW algorithm (Higgins et al. (1994) Polynucleotides Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ (Karl Nicholas) allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package (available from Accelrys, Inc., 9865 Scranton Rd., San Diego, Calif., USA). When utilizing the ALIGN program for comparing amino acid sequences, a PAM120 weight residue table, a gap length penalty of 12, and a gap penalty of 4 can be used.

Unless otherwise stated, GAP Version 10, which uses the algorithm of Needleman and Wunsch (1970) J. Mol. Biol. 48(3):443-453, will be used to determine sequence identity or similarity using the following parameters: % identity and % similarity for a nucleotide sequence using GAP Weight of 50 and Length Weight of 3, and the nwsgapdna.cmp scoring matrix; % identity or % similarity for an amino acid sequence using GAP weight of 8 and length weight of 2, and the BLOSUM62 scoring program. Equivalent programs may also be used. By “equivalent program” is intended any sequence comparison program that, for any two sequences in question, generates an alignment having identical nucleotide residue matches and an identical percent sequence identity when compared to the corresponding alignment generated by GAP Version 10.

The invention also encompasses variant polynucleotides. “Variants” of the chitin/chitosan-related nucleotide sequences include those sequences that encode a chitin/chitosan-related protein disclosed herein (e.g., ones that have been modified to increase the production of chitin and/or chitosan) but that differ conservatively because of the degeneracy of the genetic code, as well as those that are sufficiently identical as discussed above. Naturally occurring allelic variants can be identified with the use of well-known molecular biology techniques, such as polymerase chain reaction (PCR) and hybridization techniques as outlined below. Variant nucleotide sequences also include synthetically derived nucleotide sequences that have been generated, for example, by using site-directed mutagenesis but which still encode the chitin/chitosan-related proteins disclosed in the present invention as discussed below. Variant polynucleotides of the present invention encode polypeptides that are biologically active, that is, they retain the desired biological activity of the native protein, that is, chitin/chitosan-related activity. By “retains chitin/chitosan-related activity” is intended that the variant polynucleotide will encode a polypeptide that will have at least about 30%, at least about 50%, at least about 70%, or at least about 80% of the chitin/chitosan-related activity of the native protein. In some embodiments, the variant polynucleotides encode polypeptides with enhanced biological activities when compared to the native protein (including, for example, increased expression, stability, enzyme processivity or substrate affinity).

The skilled artisan will further appreciate that changes can be introduced by mutation into the nucleotide sequences of the invention thereby leading to changes in the amino acid sequence of the encoded chitin/chitosan-related protein, without altering the biological activity of the protein. Thus, variant isolated polynucleotides can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, conservative amino acid substitutions may be made at one or more predicted, preferably nonessential amino acid residues. A “nonessential” amino acid residue is a residue that can be altered from the wild-type sequence of a chitin/chitosan-related protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Amino acid substitutions may be made in nonconserved regions that retain function. In general, such substitutions would not be made for conserved amino acid residues, or for amino acid residues residing within a conserved motif, where such residues are essential for protein activity or where maintenance of a particular activity is desired (e.g., activity of the corresponding native protein). However, one of skill in the art would understand that functional variants may have minor conserved or nonconserved alterations in the conserved residues. The identification of conserved and nonconserved residues in a polypeptide can be done by various alignment and sequence comparison methods well known to those of skill in the art.

Alternatively, variant nucleotide sequences can be made by introducing mutations randomly along all or part of the coding sequence, such as by saturation mutagenesis, and the resultant mutants can be screened for ability to produce chitin and/or chitosan to identify mutants that retain activity. Following mutagenesis, the encoded protein can be expressed recombinantly, and the activity of the protein can be determined using standard assay techniques.

Using methods such as PCR, hybridization, and the like, corresponding chitin/chitosan-related sequences can be identified, such sequences having substantial identity to the sequences of the invention. See, for example, Sambrook J., and Russell, D. W. (2001) Molecular Cloning: A Laboratory Manual. (Cold Spring Harbor Laboratory Press, Cold Spring Harbor, N.Y.) and Innis, et al. (1990) PCR Protocols: A Guide to Methods and Applications (Academic Press, NY).

In a hybridization method, all or part of the chitin/chitosan-related nucleotide sequence can be used to screen cDNA or genomic libraries. Methods for construction of such cDNA and genomic libraries are generally known in the art and are disclosed in Sambrook and Russell, 2001, supra. The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known chitin/chitosan-related nucleotide sequences disclosed herein. Degenerate primers designed on the basis of conserved nucleotides or amino acid residues in the nucleotide sequences or encoded amino acid sequences can additionally be used. The probe typically comprises a region of nucleotide sequence that hybridizes under stringent conditions to at least about 12, preferably about 25, at least about 50, 75, 100, 125, 150, 175, 200, 250, 300, 350, 400, 500, 600, 700, 800, 900, 1000, 1200, 1300 consecutive nucleotides of a chitin/chitosan-related nucleotide sequence of the invention or a fragment or variant thereof. Methods for the preparation of probes for hybridization are generally known in the art and are disclosed in Sambrook and Russell, 2001, supra and Sambrook et al. (1989) Molecular Cloning: A Laboratory Manual (2d ed., Cold Spring Harbor Laboratory Press, Cold Spring Harbor, N.Y.), both of which are herein incorporated by reference.

For example, an entire chitin/chitosan-related sequence disclosed herein, or one or more portions thereof, may be used as a probe capable of specifically hybridizing to corresponding chitin/chitosan-related sequences and messenger RNAs. To achieve specific hybridization under a variety of conditions, such probes include sequences that are unique and are at least about 10 nucleotides in length, or at least about 20 nucleotides in length. Such probes may be used to amplify corresponding chitin/chitosan-related sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Cold Spring Harbor, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of polynucleotides is found in Tijssen (1993) Laboratory Techniques in Biochemistry and Molecular Biology—Hybridization with Polynucleotide Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Cold Spring Harbor, N.Y.).

C. Isolated Proteins and Variants and Fragments Thereof

Another aspect of the invention pertains to isolated biologically-active variants and fragments of chitin/chitosan-related proteins, wherein the variants and fragments, when expressed in a cell, result in an increase in the production of chitin and/or chitosan when compared to a cell that does not express the variant or fragment of the chitin/chitosan-related protein. By “chitin/chitosan-related protein” is intended a protein that is involved in the production of chitin and/or chitosan.

“Fragments” or “biologically active portions” include polypeptide fragments comprising a portion of an amino acid sequence encoding a chitin/chitosan-related protein as set forth in SEQ ID NOS:2, 4, 6, 8, 10, 12, 14, 15, 16, 17, 18, 20, 22, 24, 26, 28, 30, 32, 34, 36, 38, 40, 42 or 44, and that retains chitin/chitosan-related activity (e.g., leads to the production of chitin and/or chitosan). A biologically active portion or fragment of a chitin/chitosan-related protein can be a polypeptide that is, for example, 10, 25, 50, 100 or more amino acids in length. Such biologically active portions can be prepared by recombinant techniques and evaluated for chitin/chitosan-related activity. Methods for measuring chitin/chitosan-related activity are well known in the art and are discussed elsewhere herein. As used here, a fragment comprises at least 8 contiguous amino acids of SEQ ID NOS:2, 4, 6, 8, 10, 12, 14, 15, 16, 17, 18, 20, 22, 24, 26, 28, 30, 32, 34, 36, 38, 40, 42 or 44. The invention encompasses other fragments, however, such as any fragment in the protein greater than about 10, 20, 30, 50, 100, 150, 200, 250, 300, 350, or 400 amino acids.

By “variants” is intended proteins or polypeptides having an amino acid sequence that is at least about 60%, 65%, about 70%, 75%, 80%, 85%, or 90%, 91%, 92%, 93%, 94%, 95%, 96%, 97%, 98% or 99% identical to the amino acid sequence of SEQ ID NOS:2, 4, 6, 8, 10, 12, 14, 15, 16, 17, 18, 20, 22, 24, 26, 28, 30, 32, 34, 36, 38, 40, 42 or 44. Variants also include polypeptides encoded by a polynucleotide that hybridizes to the polynucleotide of SEQ ID NOS:1, 3, 5, 7, 9, 11, 13, 19, 21, 23, 25, 27, 29, 31, 33, 35, 37, 39, 41, or 43, or a complement thereof, under stringent conditions. Variants include polypeptides that differ in amino acid sequence due to mutagenesis. Variant polypeptides encompassed by the present invention are biologically active, that is they continue to possess the desired biological activity of the native protein, that is, production of chitin and/or chitosan. In some embodiments, the variant polypeptides have enhanced biological activities when compared to the native protein (including, for example, increased expression, stability, enzyme processivity or substrate affinity).

III. Methods

Methods of the invention involve introducing one or more chitin/chitosan-related polynucleotides into a microorganism or plant. By “introducing” is intended to present to the microorganism or plant the polynucleotide(s) in such a manner that the polynucleotide(s) gains access to the interior of a cell of the microorganism or plant. The methods of the invention do not require that a particular method for introducing a polynucleotide is used, only that the polynucleotide gains access to the interior of at least one cell of the microorganism or plant.

A. Overexpression of Chitin/Chitosan-Related Sequences

In some embodiments, a genetic modification that results in an increase in the production of chitin and/or chitosan encompasses any modification that results in an increase in gene expression, including amplification, overproduction, overexpression, or up-regulation of a gene. More specifically, reference to increasing gene expression of enzymes or other polypeptides discussed herein generally refers to any genetic modification in the microorganism or plant in question which results in increased expression of the enzymes or polypeptides, and includes (but is not limited to) reduced inhibition or degradation of the enzymes (i.e., stability) and overexpression of the enzymes. For example, gene copy number can be increased, expression levels can be increased by use of a heterologous promoter that gives higher levels of expression than that of the native promoter, or a gene can be altered by directed evolution or classical mutagenesis to increase the stability of an enzyme. Combinations of some of these modifications are also possible.

An increase in expression of a chitin/chitosan-related gene of the invention can result, for example, from the introduction of a non-native promoter upstream of at least one gene encoding an enzyme or other protein of interest in the chitin/chitosan pathway described herein. Preferably the 5′ upstream sequence of an endogenous gene is replaced by a constitutive promoter, an inducible promoter, or a promoter with optimal expression under the growth conditions used. This method is especially useful when the endogenous gene is not active or is not sufficiently active under the growth conditions used.

The promoter may be native or analogous, or foreign or heterologous, to the plant host and/or to the DNA sequence of the invention. Additionally, the promoter may be the natural sequence or alternatively a synthetic sequence. Where the promoter is “native” or “homologous” to the plant host, it is intended that the promoter is found in the native plant into which the promoter is introduced. Where the promoter is “foreign,” “heterologous” or “non-native” to the DNA sequence of the invention, it is intended that the promoter is not the native or naturally occurring promoter for the operably linked DNA sequence of the invention. “Heterologous” generally refers to the polynucleotide sequences that are not endogenous to the cell or part of the native genome in which they are present, and have been added to the cell by infection, transfection, microinjection, electroporation, microprojection, or the like. By “operably linked” is intended a functional linkage between a promoter and a second sequence, wherein the promoter sequence initiates and mediates transcription of the DNA sequence corresponding to the second sequence. Generally, operably linked means that the polynucleotide sequences being linked are contiguous and, where necessary to join two protein coding regions, contiguous and in the same reading frame.

Often, such constructs will also contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, vacuole, or Golgi apparatus, or to be secreted. For example, the gene can be engineered to contain a signal peptide to facilitate transfer of the peptide to the vacuole. By “signal sequence” is intended a sequence that is known or suspected to result in cotranslational or post-translational peptide transport across the cell membrane. By “leader sequence” is intended any sequence that when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids (including chloroplasts), mitochondria, and the like. It may also be preferable to engineer the plant expression cassette to contain an intron, such that mRNA processing of the intron is required for expression.

In some embodiments, expression can be enhanced by the introduction of 3′ or 5′ untranslated elements. By “3′ untranslated region” is intended a nucleotide sequence located downstream of a coding sequence. Polyadenylation signal sequences and other sequences encoding regulatory signals capable of affecting the addition of polyadenylic acid tracts to the 3′ end of the mRNA precursor are 3′ untranslated regions. By “5′ untranslated region” is intended a nucleotide sequence located upstream of a coding sequence.

Other upstream or downstream untranslated elements include enhancers. Enhancers are nucleotide sequences that act to increase the expression of a promoter region. Enhancers are well known in the art and include, but are not limited to, the SV40 enhancer region and the ³⁵S enhancer element.

Where appropriate, the gene(s) may be optimized for increased expression in the transformed host cell. That is, the genes can be synthesized using host cell-preferred codons for improved expression, or may be synthesized using codons at a host-preferred codon usage frequency. Generally, the GC content of the gene will be increased. See, for example, Campbell and Gowri (1990) Plant Physiol. 92: 1-11 for a discussion of host-preferred codon usage. Methods are known in the art for synthesizing host-preferred genes. See, for example, U.S. Pat. Nos. 6,320,100; 6,075,185; 5,380,831; and 5,436,391, U.S. Published Application Nos. 20040005600 and 20010003849, and Murray et al. (1989) Polynucleotides Res. 17:477-498, herein incorporated by reference.

B. Vacuole Targeting

With increased chitin production, maintenance of cellular membrane integrity is a consideration. In one embodiment of the invention, the issue of cell membrane integrity is addressed by engineering a chitin synthase such that it no longer produces chitin as an integral part of the cell wall or cell membrane, but as an insoluble polymer within the cell (much like PHA production in bacteria). This allows very high accumulation of chitin/chitosan, similar to that achieved by some PHA production strains. In another embodiment of the invention, the properties of the enzymes involved in chitin synthesis are altered such that overexpression of chitin results in secretion of the chitin as microfibrils outside the cell membrane.

In yet another embodiment of the invention, chitin/chitosan production is targeted to the vacuole, for example, by targeting the chitin and/or chitosan synthase to the vacuolar membrane. By targeting the product of chitin and/or chitosan to a vacuole, one can achieve the high level chitin/chitosan production desired while still maintaining cell viability.

As such, the present invention provides novel chimeric polypeptides comprising vacuole membrane targeting sequences operably linked to chitin/chitosan-related sequences. The term “vacuole targeting sequence” as used herein refers to a sequence operable to direct or sort a selected non-vacuolar protein to which such sequence is linked to a vacuolar membrane.

The vacuolar targeting polypeptide sequences of the invention function to direct or sort the protein products directed by the expression of genes to which they are operably linked from the cytoplasm to the vacuole of a cell. Since vacuoles have a storage function, proteins directed there remain there, continually increasing in abundance, unless subject to degradation by vacuolar proteinases. The vacuolar proteins are also isolated from the major metabolic processes in the cell and thus will not interfere with cell growth and development. Thus by targeting the synthase enzymes involved in the production of chitin and/or chitosan to the vacuolar membrane, one can alleviate problems associated with cellular membrane integrity that may result from an increased production of chitin and/or chitosan in an organism.

In the preferred embodiment, only the synthase enzymes involved in the production of chitin are targeted to the vacuole. This will result in accumulation of chitin and/or chitosan either in the vacuolar membrane or the vacuole. Chitin and/or chitosan can be collected by harvest and lysis of whole cells or by isolation and lysis of vacuole compartments by methods well known in the art.

Plant vacuolar targeting sequences include any such targeting sequences as are known in the art that effect proper vacuole targeting in plant hosts. These include polypeptides targeting barley lectin (Bednarek et al. (1990) Plant Cell 2(12):1145-1155), sweet potato sporamin (Matsuoka et al. (1990) J Biol Chem 265(32): 19750-7), tobacco chitinase (Neuhaus et al. (1991) Proc Natl Acad Sci USA 88(22):10362-6), bean phytohemagglutinin (Tague et al. (1990) Plant Cell 2(6):533-46), 2S albumin (Saalbach et al. (1996) Plant Physiol 112(3):975-85), aleurain (Holwerda et al., 1992). Vacuolar targeting in plants has been widely studied (for example see Chrispeels, 1991; Chrispeels & Raikhel, 1992; Dromboski & Raikhel, 1996; Kirsch et al., 1994; Nakamura & Matsuoka, 1993; Neilsen et at., 1996; Rusch & Kendall, 1995; Schroder et al., 1993; Vitale & Chrispeels, 1992; von Heijne, 1983). Other sequences are described, for example, in U.S. Pat. No. 5,436,394, U.S. Pat. No. 5,792,923, U.S. Pat. No. 5,360,726, U.S. Pat. No. 5,525,713 and U.S. Pat. No. 5,576,428 incorporated herein by reference.

Scott et al. (2000) J. Biol. Chem. 275(33):25840-25849 and Wang Y X, et al. (1998) J Cell Biol 140(5):1063-74 describe vacuole targeting proteins in yeast, including Vac8 and Apgl3. In fungi, vacuolar targeting proteins such as vacuolaralkaline phosphatase (ALP) and the syntaxin Vam3p are known (Cowles et al. (1997) Cell 91:109-118; Piper et al. (1997) Cell Biol. 138:531-545; Stepp et al. (1997) J. Cell Biol. 139:1761-1774; Darsow et al. (1998) J. Cell Biol. 142:913-922.

Further embodiments include vacuole membrane targeted enzymes generated as fusion proteins wherein, for example, the C-terminal region of a vacuole protein is in operable linkage with any desired protein molecule (e.g., chitin/chitosan-related polypeptides of the present invention) to ensure that the proteins associated with those peptide fragments are directed specifically into the vacuole (see, for example, U.S. Pat. No. 6,054,637). When membrane insertion is desired (such as for chitin synthase), fusion proteins are generated using vacuole membrane proteins. Vacuole membrane proteins are known in the art and include, for example, sec17, phytepsin, plasmepsin, the FYVE domain or Vps27, and the PX domain of vacuole morphogenesis protein VAM7.

C. Modification of Enzyme Activity

The ability to engineer efficient enzymes into a microorganism or plant, and to overcome any internal down-regulation of chitin biosynthesis is important for high-level production of chitin/chitosan. Development of very efficient enzymes is likely to be a key to this success. The development of enzymes with high rates of reaction and a strong affinity for their substrates allows a modest protein expression to achieve very substantial metabolic flow from fructose-6-phosphate to chitosan.

Thus, in one embodiment of the invention, chitin/chitosan yield is optimized by assessing the fermentative production of a microorganism, for example, Aspergillus niger, in culture using methods known in the art. Assays for detecting chitin/chitosan synthesis, as well as for detecting improvements in chitosan synthesis are also well known in the art and discussed elsewhere herein. Mutant strains with improved chitin/chitosan yield can be isolated after mutagenesis and screening techniques that are known to one of skill in the art. In addition, the individual enzymes can be modified by using techniques such as in vitro mutagenesis. Modified strains can be engineered with a heterologous or mutated chitin synthase, GFA, and/or chitin deacetylase. All desired constructs can then be integrated into the genome of the microorganism or plant by known molecular biology techniques.

Therefore, a gene encoding modified enzyme or other protein useful in the present invention can be a mutated (i.e., genetically modified) gene, for example, and can be produced by any suitable method of genetic modification. For example, a recombinant polynucleotide encoding the enzyme can be modified by any method for inserting, deleting, and/or substituting nucleotides, such as by error-prone PCR or directed evolution strategies. In error-prone PCR methods, the gene is amplified under conditions that lead to a high frequency of misincorporation errors by the DNA polymerase used for the amplification. As a result, a high frequency of mutations is obtained in the PCR products. Additionally, computer-based protein engineering can be used for genetic modification of a gene. See for example, Maulik et al. (1997) Molecular Biotechnology: Therapeutic Applications and Strategies (Wiley-Liss, Inc., Wilmington, Del.) which is incorporated herein by reference in its entirety.

In other embodiments of the invention, chitin and/or chitosan are produced using plants or microorganisms that have modified chitin synthase, GFA, and/or chitin deacetylase with improved processivity, increased reaction velocity, and/or a modified Kₘ for a substrate. By improving the catalytic efficiency of the key catabolic enzymes, massive redirection of sugar towards synthesis of chitin may be acquired. By developing highly efficient enzymes for the generation of precursor, the synthesis of chitin, and the deacetylation of nascent chitin to chitosan, and by manipulating the genetic background of microorganisms or plants, in combination with traditional mutagenesis and improvement techniques, one can develop a plant or microbial strain with high chitosan production. In another embodiment, the modified or mutated chitin synthase has a substrate preference for UDP-glucosamine-1-phosphate (UDP-Gln). A substrate preference of chitin synthase for UDP-Gln will allow increased production of chitosan compared to chitin.

In another embodiment, enzymes and polypeptides of the chitin/chitosan production pathway are modified (e.g., using classical mutagenesis or directed evolution strategies) to acquire enzymes with extremely high activity and processivity. Most enzymes are not extremely efficient in either binding or catalyzing reactions with their substrates. In fact, many important and industrially valuable enzymes are, in fact, quite poor at performing catalysis. Kₘ's of over 1 mM for substrate are quite typical for such enzymes. For example, chitin synthase from the fungus Mucor rouxii has a Kₘ for its substrate, NAc-Gln, of 1.8 mM (Davis and Garcia (1984) Biochemistry 23:1065-1073).

Kₘs on the order of 30-50 μM are desirable. It is also likely that overall reaction rate (V) can be improved substantially. For example, many hydrolytic enzymes (including synthases) have a poor V due to poor reaction of water with the enzyme-product intermediate. Mutations that improve the water accessibility of the second reaction result in substantial catalytic efficiency. Furthermore, improvements in protein folding are often obtained, and result in further improvements in reaction velocity (V). Thus, the prognosis for large improvement in catalytic efficiency of a hydrolytic or synthetic enzyme is quite good.

Development of a chitin synthase that efficiently catalyzes synthesis of chitin, a chitin deacetylase that rapidly deacetylates nascent chitin chains to yield chitosan, and a GFA that rapidly shunts fructose 6-phosphate to generation of UDP-NAc-Gln is desirable. The result of this is an enzyme system that efficiently converts fructose-6-phosphate to chitosan. Using directed evolution (for example, by the in vitro mutagenesis technique described below), the possibility also exists to directly evolve a chitin synthase to utilize UDP-glucosamine-1-phosphate as a substrate, and to produce chitosan directly (“a chitosan synthase”). This system can then be introduced into a yeast, for example Saccharomyces cerevisiae, a fungus, for example Aspergillus niger, a bacteria, for example E. coli, or a plant to serve as a chitosan factory. In some embodiments, the “chitosan synthase” utilizes both UDP-NAc-Gln and UDP-glucosamine-1-phosphate as substrate. Such an enzyme is capable of de novo synthesis of both chitin and chitosan, depending on the availability of substrate.

The processivity of a polymer-forming enzyme (i.e., the number of monomers incorporated before dissociating from the polymer) is a reflection of a number of factors, mainly the rate of dissociation (K_{off}), the rate of binding to polymer vs. monomer (starting a new chain), and the total monomer incorporated per polymerization “round” (number of polymerizations carried out on average between binding and dissociation). Directed evolution strategies can allow identification of mutant synthases with a higher degree of processivity by alteration in any of these factors. A more processive enzyme is likely to extrude a longer chain length chitin polymer, leading to a higher molecular weight chitosan. Secondly, it is likely that a highly processive synthase will create very long chains of chitin that will accumulate in places other than the cell wall, and ideally be extruded into the medium. This can allow very high chitin levels to be achieved, with reduced possibility of a deleterious effect to the cells.

In general, directed evolution strategies may provide improvements in the velocity of reaction (V), often through either improved folding or by increasing the relative number of available active sites. For example, amino acid sequence variants of the targeted enzymes can be prepared by mutations in the polynucleotides encoding the amino acid sequence. This may be accomplished by one of several forms of mutagenesis and/or in directed evolution. In some aspects, the changes encoded in the amino acid sequence will not substantially affect the function of the protein. However, the ability of the enzymes to produce chitin/chitosan may be improved by the use of such techniques. For example, one may express the target enzyme in host cells that exhibit high rates of base misincorporation during DNA replication, such as XL-1 Red (Stratagene, La Jolla, Calif.). After propagation in such strains, one can isolate the enzyme DNA (for example by preparing plasmid DNA, or by amplifying by PCR and cloning the resulting PCR fragment into a vector), culture the enzyme mutations in a non-mutagenic strain, and identify mutated enzyme genes with enhanced activity, for example, by performing an assay to test for enzymes with increased processivity, or altered Kₘ for a substrate. Methods for assaying enzyme activity are discussed elsewhere herein.

A high efficiency chitin deacetylase is needed to deacetylate chitin oligomers at a rate matching the synthesis of chitin. Thus, as more efficient chitosan synthases are developed, the activity of chitin deacetylase will also need to be improved. Chitin deacetylase has a Kₘ for oligochitin of approximately 40 μM (Tokuyasu et al. (1999) FEBS Letters 458:23-26). Thus, one would expect catalytic improvements to not only improve this affinity, but also improve the overall rate of reaction, which has a V of ˜50 s⁻¹ (op cit).

In another embodiment of the invention, chitin synthesis yields are dramatically improved by increasing the flow of precursor materials to chitin synthase. Chitin yield appears to be limited (regulated) by the availability of its substrate (UDP-NAc-Gln). The key enzyme in the regulation of UDP-NAc-Gln production appears to be the glucosamine-fructose-6 phosphate aminotransferase (GFA). This enzyme is a key candidate for directed evolution for several reasons. Increased expression of GFA results in higher chitin yield in yeast. GFA1 from yeast has a Kₘ for its two substrates of 300 μM (glutamine) and 700-800 μM (fructose-6-phosphate). Genetic and biochemical evidence suggests that synthesis of glucosamine 6-phosphate by GFA is a key commitment step in amino sugar biosynthesis. By increasing the shunting of fructose-6phosphate to the formation of UDP-NAc-Gln, one should increase chitin yields dramatically.

Chitosan is currently derived from chitin by either action of chitin deacetylase upon nascent chitin chains, or chemical deacetylation after cell harvest. The application of directed evolution technologies opens the possibility to develop a “chitosan synthase” that will directly synthesize glutamine polymers. FIG. 1 shows the steps involved in synthesis of amino sugars in most biological systems. It is important to note that the pathways for synthesis of UDP-Nac-Gln and UDP glucosamine are linked at several places, offering several opportunities to develop a chitin-synthesizing strain into a chitosan-synthesizing strain. The ultimate key to this approach is to improve the specificity of the chitin synthase to favor incorporation of UDP-Gln over UDP-NAc-Gln.

D. Expression in Bacterial Systems

Another approach for producing chitin/chitosan involves development of a bacterial chitin/chitosan production system. Achieving production of chitin/chitosan in E. coli, or a gram-positive bacterium, will lead to a cost effective production system, and may allow accumulation of chitin at levels matching PHA production in bacteria (up to 70% of cell weight). In one embodiment, this approach involves genetically modifying a bacterium to express a bacterial chitin synthase that has been modified (e.g., by classical mutagenesis or directed evolution strategies) to have increased processivity. Previous work has shown that the NodC gene from Rhizobium is capable of synthesizing gram-scale quantities of chitin oligomer in E. coli (Bettler et al. (1999) Glycoconj J 16(3):205-212). However, the natural preference of this enzyme is to produce short oligomers four to five sugars long. By genetically modifying the bacterial chitin synthase to increase its processivity, longer-chained chitin polymers can be produced, resulting in a gram-scale chitin production system in bacteria.

In another embodiment, the present invention describes a bacterium that expresses one or more heterologous genes involved in the production of chitin and/or chitosan. Further embodiments include expression by the bacterium of one or more genes involved in the production of chitin and/or chitosan that has been modified according the methods of the present invention, wherein the modification results in an increase in the production of chitin and/or chitosan in the bacterium or the media in which the bacterium is cultured/fermented.

Bacteria (such as E. coli) produce UDP-Nac-Gln and incorporate it as a component of their cell walls. Thus, a chitosan production system based on bacterial expression of a heterologous chitin synthase in tandem with a heterologous chitin deacetylase would be desirable. Such a system would be attractive as bacteria are easy to ferment, and many strains, such as Bacillus strains, can yield high biomass levels in fermentation. Bacterial expression would also allow tight control of expression of chitosan production.

Thus, in another embodiment, a bacterium is genetically modified to express a chitin synthase that allows production of chitin as an insoluble polymer within at least one cell of the microorganism. By “allows production of chitin as an insoluble polymer” is intended that the enzyme has been modified such that it is capable of synthesizing or contributing to the synthesis of chitin that is not incorporated into or bound to the cell wall or cell membrane of the microorganism in which it is produced. For example, in one such embodiment, the genetically modified bacterium comprises a chitin synthase from the chlorovirus CVK2 (a virus that infects the algae Chlorella, represented in SEQ ID NO:33 and 34), which reportedly lacks obvious membrane-spanning domains. Alternatively, a eukaryotic chitin synthase (such as the chitin synthase of Mucor rouxii or an Aspergillus niger chitin synthase) is engineered for expression in the bacterial membrane. In some embodiments, the insoluble polymer is extruded from the cell.

E. In vitro Production of Chitin and/or Chitosan

A final approach involves an in vitro production system for chitosan. An in vitro system would allow tight control of chitosan synthesis. This will allow generation of very high purity material, which may be advantageous for certain applications, such as medical applications. It will also allow very tight control of the chain length and amount of acetylation and allow facile alteration of the polymer to suit different high value applications. Further, in vitro systems open the possibility to easily generate interesting copolymers with other UDP-sugars. Development of highly efficient enzymes for chitin production (synthases, deacetylases, and precursor generation enzymes) would open the door to such possibilities.

In this manner, the present invention provides an in vitro chitosan production system that has been developed using a combination of enzymes and permeabilized cells. This system utilizes a combination of eukaryotic cells to produce an easily purified (His-tagged) chitin synthase, and at least one bacterium as a source of NAc-Gln. In one embodiment, E. coli is used as the UDP-NAc-Gln production strain, and the chitin deacetylase is also produced in E. coli. In other embodiments, chitin synthase is expressed in yeast, and chitin deacetylase is expressed in E. coli. Both of these enzymes are then purified and linked to a resin such as agarose beads. The bacterium that has been metabolically engineered to serve as a UDP-NAc-Gln source is then permeabilized. The enzymes and permeabilized cells are mixed together in vitro, and the resulting chitin/chitosan is isolated.

F. Directed Evolution of Chitin/Chitosan-Related Enzymes

In one embodiment, a gene encoding a modified enzyme or other protein useful in the present invention can be produced using directed evolution strategies. Directed evolution of enzymes involved in the production of chitin and/or chitosan can be accomplished by generating random or targeted mutations in the protein coding sequence and screening the mutant library for functional improvements (e.g., improvement in the production of chitin and/or chitosan). With saturation mutagenesis, it is possible to create a library of mutants containing all possible mutations at one or more pre-determined target positions in a gene sequence. This method is used in directed evolution experiments to expand the number of amino acid substitutions accessible by random mutagenesis. The resulting mutants can then be screened to identify mutations which result in an increase in the production of chitin and/or chitosan.

G. Microbial Culture/Fermentation

As noted above, in the method for production of chitin and/or chitosan of the present invention, a microorganism or plant having a genetically modified chitin/chitosan pathway is cultured in a fermentation medium for production of chitin and/or chitosan. An appropriate, or effective, fermentation medium refers to any medium in which a genetically modified microorganism or plant of the present invention, when cultured, is capable of producing (accumulating) chitin and/or chitosan. Such a medium is typically an aqueous medium comprising assimilable carbon, nitrogen and phosphate sources. Such a medium can also include appropriate salts, minerals, metals and other nutrients. For example, a minimal-salts medium containing glucose, fructose, lactose, glycerol or a mixture of two or more different compounds as the sole carbon source is preferably used as the fermentation medium. The use of a minimal-salts-glucose medium is the most preferred medium for the chitin and/or chitosan fermentation and it will also facilitate recovery and purification of the products. One of ordinary skill in the art can readily determine the optimum culture medium for culturing a particular organism.

Sufficient oxygen must be added to the medium during the course of the fermentation to maintain cell growth during the initial growth phase and to maintain metabolism, and chitin and/or chitosan production. Oxygen is conveniently provided by agitation and aeration of the medium. Conventional methods, such as stirring or shaking, may be used to agitate and aerate the medium. The oxygen concentration of the medium can be monitored by conventional methods, such as with an oxygen electrode. Other sources of oxygen, such as undiluted oxygen gas and oxygen gas diluted with inert gas other than nitrogen, can be used.

Microorganism or plants of the present invention can be cultured in conventional fermentation bioreactors. The microorganism or plants can be cultured by any fermentation process which includes, but is not limited to, batch, fed-batch, cell recycle, and continuous fermentation. Preferably, microorganism or plants of the present invention are grown by batch or fed-batch fermentation processes.

Fermentation conditions can include culturing the microorganism or plants of the invention at any temperature between about 20° C. and about 40° C., in whole increments (i.e., 21° C., 22° C., etc.). It is noted that the optimum temperature for growth and chitin and/or chitosan production by a microorganism or plant of the present invention can vary according to a variety of factors. For example, the selection of a particular promoter for expression of a recombinant polynucleotide in the microorganism or plant can affect the optimum culture temperature. One of ordinary skill in the art can readily determine the optimum growth and chitin and/or chitosan production temperature for any microorganism or plant of the present invention using standard techniques.

In addition, suitable fermentation mediums and culture conditions for microorganism or plants of the present invention are described in detail in U.S. Pat. No. 6,372,457 and PCT Publication No. WO 2004/003175 A2, as well as in Berka and Barnett (1989) Biotechnol Adv 7(2):127-154.

H. Collection of Chitin/Chitosan

In another embodiment of the present invention, methods to collect, recover and purify chitin and chitosan from plant or microbial biomass produced by the methods of the present invention are included in the method of chitin or chitosan production. These methods are based on those described previously in U.S. Pat. No. 4,806,474; International Publication No. WO 01/68714 and other publications (e.g., Synowiecki and Al-Khateeb (2003) Crit Rev Food Sci Nutr 43(2):145-171; Pochanavanich and Suntomsuk (2002) Lett Appl Microbiol 35(1):17-21; Amorim et al. (2001) Braz JMicrobiol 32:20-23). Each of these publications is incorporated herein by reference in its entirety.

To “collect” a product such as chitin and/or chitosan can simply refer to collecting the biomass from a fermentation bioreactor, microbiological isolates, or plant extracts, and need not imply additional steps of separation, recovery, or purification. The term “recovering” or “recover,” as used herein with regard to recovering chitin and/or chitosan products, refers to performing additional processing steps on the plant or microbial biomass to obtain chitin and/or chitosan at any level of purity. These steps can be followed by further purification steps. For example, chitin and/or chitosan can be recovered from the biomass by a technique that includes, but is not limited to, the following steps: treatment of microorganism or plant cells with a hot alkaline solution, collection and washing of the remaining solids containing chitin or chitosan, resuspension of the washed solids in an acidic solution to solubilize the chitin or chitosan, and precipitation of the chitin or chitosan. Chitin and/or chitosan are preferably recovered in substantially pure forms. As used herein, “substantially pure” refers to a purity that allows for the effective use of the chitin and/or chitosan as a compound for commercial sale or use. In one embodiment, the chitin and/or chitosan products are preferably separated from the production organism and other fermentation medium constituents. Methods to accomplish such separation are well known in the art and are referenced above.

Preferably, by the method of the present invention, at least about 25% of product (i.e., chitin and/or chitosan) by weight is recovered from the plant or microbial biomass and/or collected as a dry weight of chitin and/or chitosan within the plant or microbial biomass. More preferably, by the method of the present invention, at least about 30%, at least about 40%, 45%, 50%, 60%, 70%, 75%, 80%, 85%, 95%, 96%, 97%, 98%, 99% or up to 100% of the product is recovered.

Preferably, using the method of the present invention, the microorganism or plant produces at least about 0.5% of its total biomass by dry weight as chitin or chitosan, at least about 1%, at least about 2%, 3%, 4%, 5%, 7%, 10%, 20%, 30%, 40% or higher.

In another embodiment, using the method of the present invention, the microorganism or plant produces at least about 0.1 gram of chitin or chitosan per liter of fermentation medium in which the biomass producing the chitin or chitosan is cultured, at least about 0.2 g/L, at least about 0.3 g/L, at least about 0.4, 0.5, 7.5, 10, 15, 20, 25, 50, 100, 200 g/L or higher.

I. Transfection of Microorganisms and Plants

In another aspect, the polynucleotides of the invention are expressed in a host cell to generate a genetically modified microorganism or plant. The host cell can include: (1) a host cell that does not express the particular enzyme or protein, or (2) a host cell that does express the particular enzyme or protein, wherein the introduced recombinant polynucleotide changes or enhances the activity of the enzyme or other protein in the microorganism or plant. The present invention intends to encompass any genetically modified microorganism or plant, wherein the microorganism or plant comprises at least one modification suitable for a fermentation process to produce chitin and/or chitosan according to the present invention.

A genetically modified microorganism or plant can be modified by recombinant technology, such as by introduction of an isolated polynucleotide into a microorganism or plant. For example, a genetically modified microorganism or plant can be transfected with a recombinant polynucleotide encoding a protein of interest, such as a protein for which increased expression is desired. The transfected polynucleotide can remain extrachromosomal or can integrate into one or more sites within a chromosome of the transfected (i.e., recombinant) host cell in such a manner that its ability to be expressed is retained. Preferably, once a host cell of the present invention is transfected with a polynucleotide, the polynucleotide is integrated into the host cell genome. A significant advantage of integration is that the polynucleotide is stably maintained in the cell. In a preferred embodiment, the integrated polynucleotide is operatively linked to a transcription control sequence (described above) that can be induced to control expression of the polynucleotide.

A polynucleotide can be integrated into the genome of the host cell either by random or targeted integration. Such methods of integration are known in the art. A genetically modified microorganism can also be produced by introducing polynucleotides into a recipient cell genome by a method such as by using a transducing bacteriophage. The use of recombinant technology and transducing bacteriophage technology to produce several different genetically modified microorganisms of the present invention is known in the art.

A recombinant cell is preferably produced by transforming a host cell (e.g., a yeast or other fungal cell) with one or more recombinant molecules, each comprising one or more polynucleotides operatively linked to an expression vector containing one or more transcription control sequences. The phrase “operatively linked” refers to insertion of a polynucleotide into an expression vector in a manner such that the molecule can be expressed when transformed into a host cell. As used herein, an expression vector is a DNA or RNA vector that is capable of transforming a host cell and of affecting expression of a specified polynucleotide. Preferably, the expression vector is also capable of replicating within the host cell. In the present invention, expression vectors are typically plasmids. Expression vectors of the present invention include any vectors that function (i.e., direct gene expression) in a host cell. Preferred host cells include, but are not limited to any suitable bacterium, a protist, a microalgae, a fungus, or other microbe, with fungi being particularly preferred.

Transformation of bacterial cells is accomplished by one of several techniques known in the art, including but not limited to electroporation or chemical transformation (see, for example, Ausubel, ed. (1994) Current Protocols in Molecular Biology, John Wiley and Sons, Inc., Indianapolis, Ind.). Markers conferring resistance to toxic substances are useful in identifying transformed cells (having taken up and expressed the test DNA) from non-transformed cells (those not containing or not expressing the test DNA).

Transformation of plant cells can be accomplished in similar fashion. By “plant” is intended whole plants, plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen). “Transgenic plants” or “transformed plants” or “stably transformed” plants or cells or tissues refer to plants that have incorporated or integrated exogenous polynucleotide sequences or DNA fragments into the plant cell. By “stable transformation” is intended that the nucleotide construct introduced into a plant integrates into the genome of the plant and is capable of being inherited by progeny thereof.

The chitin/chitosan-related genes of the invention may be modified to obtain or enhance expression in plant cells. The chitin/chitosan-related sequences of the invention may be provided in expression cassettes for expression in the plant of interest. “Plant expression cassette” includes DNA constructs that are capable of resulting in the expression of a protein from an open reading frame in a plant cell. The cassette will include in the 5′-3′ direction of transcription, a transcriptional initiation region (i.e., promoter) operably-linked to a DNA sequence of the invention, and a transcriptional and translational termination region (i.e., termination region) functional in plants. The cassette may additionally contain at least one additional gene to be cotransformed into the organism, such as a selectable marker gene. Alternatively, the additional gene(s) can be provided on multiple expression cassettes. Such an expression cassette is provided with a plurality of restriction sites for insertion of the chitin/chitosan-related sequence to be under the transcriptional regulation of the regulatory regions.

Often, such constructs will also contain 5′ and 3′ untranslated regions. Such constructs may contain a “signal sequence” or “leader sequence” to facilitate co-translational or post-translational transport of the peptide of interest to certain intracellular structures such as the chloroplast (or other plastid), endoplasmic reticulum, vacuole, or Golgi apparatus, or to be secreted. For example, the gene can be engineered to contain a signal peptide to facilitate transfer of the peptide to the vacuole. By “signal sequence” is intended a sequence that is known or suspected to result in cotranslational or post-translational peptide transport across the cell membrane. By “leader sequence” is intended any sequence that when translated, results in an amino acid sequence sufficient to trigger co-translational transport of the peptide chain to a sub-cellular organelle. Thus, this includes leader sequences targeting transport and/or glycosylation by passage into the endoplasmic reticulum, passage to vacuoles, plastids (including chloroplasts), mitochondria, and the like. It may also be preferable to engineer the plant expression cassette to contain an intron, such that mRNA processing of the intron is required for expression.

Typically this “plant expression cassette” will be inserted into a “plant transformation vector.” By “transformation vector” is intended a DNA molecule that is necessary for efficient transformation of a cell. Such a molecule may consist of one or more expression cassettes, and may be organized into more than one “vector” DNA molecule. For example, binary vectors are plant transformation vectors that utilize two non-contiguous DNA vectors to encode all requisite cis- and trans-acting functions for transformation of plant cells (Hellens and Mullineaux (2000) Trends in Plant Science 5:446-451). “Vector” refers to a polynucleotide construct designed for transfer between different host cells. “Expression vector” refers to a vector that has the ability to incorporate, integrate and express heterologous DNA sequences or fragments in a foreign cell.

This plant transformation vector may be comprised of one or more DNA vectors needed for achieving plant transformation. For example, it is a common practice in the art to utilize plant transformation vectors that are comprised of more than one contiguous DNA segment. These vectors are often referred to in the art as “binary vectors.” Binary vectors as well as vectors with helper plasmids are most often used for Agrobacterium-mediated transformation, where the size and complexity of DNA segments needed to achieve efficient transformation is quite large, and it is advantageous to separate functions onto separate DNA molecules. Binary vectors typically contain a plasmid vector that contains the cis-acting sequences required for T-DNA transfer (such as left border and right border), a selectable marker that is engineered to be capable of expression in a plant cell, and a “gene of interest” (a gene engineered to be capable of expression in a plant cell for which generation of transgenic plants is desired). Also present on this plasmid vector are sequences required for bacterial replication. The cis-acting sequences are arranged in a fashion to allow efficient transfer into plant cells and expression therein. For example, the selectable marker gene and the gene of interest are located between the left and right borders. Often a second plasmid vector contains the trans-acting factors that mediate T-DNA transfer from Agrobacterium to plant cells. This plasmid often contains the virulence functions (Vir genes) that allow infection of plant cells by Agrobacterium, and transfer of DNA by cleavage at border sequences and vir-mediated DNA transfer, as is understood in the art (Hellens and Mullineaux (2000) Trends in Plant Science, 5:446-451). Several types of Agrobacterium strains (e.g. LBA4404, GV3101, EHA101, EHA105, etc.) can be used for plant transformation. The second plasmid vector is not necessary for transforming the plants by other methods such as microprojection, microinjection, electroporation, polyethylene glycol, etc.

J. Plant Transformation

Methods of the invention involve introducing a nucleotide construct into a plant. By “introducing” is intended to present to the plant the nucleotide construct in such a manner that the construct gains access to the interior of a cell of the plant. The methods of the invention do not require that a particular method for introducing a nucleotide construct to a plant is used, only that the nucleotide construct gains access to the interior of at least one cell of the plant. Methods for introducing nucleotide constructs into plants are known in the art including, but not limited to, stable transformation methods, transient transformation methods, and virus-mediated methods.

In general, plant transformation methods involve transferring heterologous DNA into target plant cells (e.g. immature or mature embryos, suspension cultures, undifferentiated callus, protoplasts, etc.), followed by applying a maximum threshold level of appropriate selection (depending on the selectable marker gene) to recover the transformed plant cells from a group of untransformed cell mass. Explants are typically transferred to a fresh supply of the same medium and cultured routinely. Subsequently, the transformed cells are differentiated into shoots after placing on regeneration medium supplemented with a maximum threshold level of selecting agent. The shoots are then transferred to a selective rooting medium for recovering rooted shoot or plantlet. The transgenic plantlet then grow into mature plant and produce fertile seeds (e.g. Hiei et al. (1994) The Plant Journal 6:271-282; Ishida et al. (1996) Nature Biotechnology 14:745-750). Explants are typically transferred to a fresh supply of the same medium and cultured routinely. A general description of the techniques and methods for generating transgenic plants are found in Ayres and Park (1994) Critical Reviews in Plant Science 13:219-239 and Bommineni and Jauhar (1997) Maydica 42:107-120. Since the transformed material contains many cells, both transformed and non-transformed cells are present in any piece of subjected target callus or tissue or group of cells. The ability to kill non-transformed cells and allow transformed cells to proliferate results in transformed plant cultures. Often, the ability to remove non-transformed cells is a limitation to rapid recovery of transformed plant cells and successful generation of transgenic plants. Molecular and biochemical methods can then be used to confirm the presence of the integrated heterologous gene of interest in the genome of transgenic plant.

Generation of transgenic plants may be performed by one of several methods, including but not limited to introduction of heterologous DNA by Agrobacterium into plant cells (Agrobacterium-mediated transformation), bombardment of plant cells with heterologous foreign DNA adhered to particles, and various other non-particle direct-mediated methods (e.g. Hiei et al. (1994) The Plant Journal 6:271-282; Ishida et al. (1996) Nature Biotechnology 14:745-750; Ayres and Park (1994) Critical Reviews in Plant Science 13:219-239; Bommineni and Jauhar (1997) Maydica 42:107-120) to transfer DNA.

Methods for transformation of chloroplasts are known in the art. See, for example, Svab et al. (1990) Proc. Natl. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-bome transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

K. Evaluation of Plant Transformation

Following introduction of heterologous foreign DNA into plant cells, the transformation or integration of heterologous gene in the plant genome is confirmed by various methods such as analysis of polynucleotides, proteins and metabolites associated with the integrated gene.

PCR analysis is a rapid method to screen transformed cells, tissue or shoots for the presence of incorporated gene at the earlier stage before transplanting into the soil (Sambrook and Russell (2001) Molecular Cloning: A Laboratory Manual, Cold Spring Harbor Laboratory Press, Cold Spring Harbor, N.Y.). PCR is carried out using oligonucleotide primers specific to the gene of interest or Agrobacterium vector background, etc.

Plant transformation may be confirmed by Southern blot analysis of genomic DNA (Sambrook and Russell, 2001, supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled ³²P target DNA fragments to confirm the integration of the introduced gene in the plant genome according to standard techniques (Sambrook and Russell, 2001, supra).

In Northern analysis, RNA is isolated from specific tissues of transformant, fractionated in a formaldehyde agarose gel, blotted onto a nylon filter according to standard procedures that are routinely used in the art (Sambrook and Russell, 2001, supra). Expression of RNA encoded by polynucleotide sequences involved in the production of chitin and/or chitosan is then tested by hybridizing the filter to a radioactive probe derived from a polynucleotide of the invention, by methods known in the art (Sambrook and Russell, 2001, supra)

Western blot and biochemical assays and the like may be carried out on the transgenic plants to determine the presence of protein encoded by the chitin/chitosan-related gene by standard procedures (Sambrook and Russell, 2001, supra) using antibodies that bind to one or more epitopes present on the chitin/chitosan-related protein.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. A microorganism comprising at least one heterologous polynucleotide sequence selected from the group consisting of:
(a) (i) a polynucleotide having at least 90% sequence identity to the nucleotide sequence set forth in SEQ ID NOS:7, 9, 11, or 13;
(ii) a polynucleotide that hybridizes to the complement of the polynucleotide of (a)(i) under high stringency conditions;
(iii) a polynucleotide encoding a polypeptide having at least 90% sequence identity to SEQ ID NO: 8, 10, 12, or 14;
wherein said polynucleotide of (a)(i), (a)(ii), and (a)(iii) encodes a polypeptide with increased glutamine-fructose-6-phosphate aminotransferase (GFA) activity compared to a native GFA;
(b) (i) a polynucleotide having at least 90% sequence identity to the nucleotide sequence set forth in SEQ ID NOS:1, 3, 5, 19, 21, 23, 25, 27, 29, 31, 33, or 43;
(ii) a polynucleotide that hybridizes to the complement of the polynucleotide of (b)(i) under high stringency conditions;
(iii) a polynucleotide encoding a polypeptide having at least 90% sequence identity to SEQ ID NO: 2, 4, 6, 20, 22, 24, 26, 28, 30, 32, 34 or 44;
wherein said polynucleotide of (b)(i), (b)(ii), and (b)(iii) encodes a polypeptide with increased chitin synthase activity compared to a native chitin synthase; and,
(c) (i) a polynucleotide having at least 90% sequence identity to the nucleotide sequence set forth in SEQ ID NOS: 35, 37, 39, or 41;
(ii) a polynucleotide that hybridizes to the complement of the polynucleotide of (c)(i) under high stringency conditions;
(iii) a polynucleotide encoding a polypeptide having at least 90% sequence identity to SEQ ID NO: 36, 38, 40, or 42;
wherein said polynucleotide of (c)(i), (c)(ii), and (c)(iii) encodes a polypeptide with increased chitin deacetylase activity compared to a native chitin deacetylase.

2. The microorganism of claim 1, wherein said microorganism is a fungus.

3. The fungus of claim 2, wherein said polynucleotide of (a) encodes a polypeptide with a lower Km for a substrate compared to a native fungal GFA.

4. The fungus of claim 3, wherein said Km is about 30 μM to about 50 μM.

5. The fungus of claim 2, wherein said polynucleotide of (b) encodes a polypeptide with a lower Kₘ for a substrate compared to a native fungal chitin synthase.

6. The fungus of claim 2, wherein said polynucleotide of (c) encodes a polypeptide with a lower Kₘ for a substrate compared to a native fungal chitin deacetylase.

7. The fungus of claim 2, wherein said fungus is Aspergillus niger or Rhizopus oryza.

8. The fungus of claim 2, wherein said fungus is a yeast.

9. The microorganism of claim 1, wherein said microorganism is a bacterium.

10. The bacterium of claim 9, wherein said bacterium is E. coli.

11. The microorganism of claim 1, wherein said polynucleotide of (b) encodes a polypeptide with increased processivity compared to a native chitin synthase.

12. The microorganism of claim 1, wherein said polynucleotide of (b) encodes a polypeptide with increased reaction velocity compared to a native chitin synthase.

13. A microorganism comprising a heterologous polynucleotide encoding a chitin synthase enzyme that allows production of chitin as an insoluble polymer within at least one cell of the microorganism.

14. The microorganism of claim 13, wherein said polynucleotide is selected from the group consisting of:
(a) (i) a polynucleotide comprising the nucleotide sequence set forth in SEQ ID NO:43;
(ii) a polynucleotide having at least 90% sequence identity to the nucleotide sequence set forth in SEQ ID NOS:43;
(iii) a polynucleotide that hybridizes to the complement of the polynucleotide of (a)(i) under high stringency conditions;
(iv) a polynucleotide encoding the polypeptide set forth in SEQ ID NO:44; or,
(v) a polynucleotide encoding a polypeptide having at least 90% sequence identity to SEQ ID NO:44; and,
(b) (i) a polynucleotide comprising the nucleotide sequence set forth in SEQ ID NO:1, 3, 5, 19, 21, 23, 25, 27, 29, 31, or 33;
(ii) a polynucleotide having at least 80% sequence identity to the nucleotide sequence set forth in SEQ ID NOS:1, 3, 5, 19, 21, 23, 25, 27, 29, 31, or 33;
(iii) a polynucleotide that hybridizes to the complement of the polynucleotide of (b)(i) under high stringency conditions;
(iv) a polynucleotide encoding the polypeptide set forth in SEQ ID NO: 2, 4, 6, 20, 22, 24, 26, 28, 30, 32, or 34; or,
(v) a polynucleotide encoding a polypeptide having at least 80% sequence identity to SEQ ID NO: 2, 4, 6, 20, 22, 24, 26, 28, 30, 32, or 34;
wherein said polynucleotide of (b)(i), (b)(ii), (b)(iii), (b)(iv) and (b)(iv) encodes a polypeptide that allows production of chitin as an insoluble polymer within at least one cell of the microorganism.

15. The microorganism of claim 13, further comprising at least one heterologous polynucleotide sequence selected from the group consisting of:
(a) (i) a polynucleotide comprising the nucleotide sequence set forth in SEQ ID NO: 7, 9, 11, or 13;
(ii) a polynucleotide having at least 90% sequence identity to the nucleotide sequence set forth in SEQ ID NOS: 7, 9, 11, or 13;
(iii) a polynucleotide that hybridizes to the complement of the polynucleotide of (a)(i) or (a)(ii) under high stringency conditions;
(iv) a polynucleotide encoding the polypeptide set forth in SEQ ID NO: 8, 10, 12, or 14; or,
(v) a polynucleotide encoding a polypeptide having at least 90% sequence identity to SEQ ID NO: 8, 10, 12, or 14.
(b) (i) a polynucleotide comprising the nucleotide sequence set forth in SEQ ID NO: 35, 37, 39, or 41;
(ii) a polynucleotide having at least 90% sequence identity to the nucleotide sequence set forth in SEQ ID NOS: 35, 37, 39, or 41;
(iii) a polynucleotide that hybridizes to the complement of the polynucleotide of (b)(i) or (b)(ii) under high stringency conditions;
(iv) a polynucleotide encoding the polypeptide set forth in SEQ ID NO: 36, 38, 40, or 42; or,
(v) a polynucleotide encoding a polypeptide having at least 90% sequence identity to SEQ ID NO: 36, 38, 40, or 42.

16. The microorganism of claim 15, wherein said polynucleotide of (a)(i), (a)(ii), (a)(iii), (a)(iv), and (a)(v) encodes a polypeptide with increased GFA activity compared to a native GFA.

17. The microorganism of claim 15, wherein said polynucleotide of (b)(i), (b)(ii), (b)(iii), (b)(iv) and (b)(v) encodes a polypeptide with increased chitin deacetylase activity compared to a native chitin deacetylase.

18. The microorganism of claim 13, wherein said chitin comprises up to 70% of the total dry weight of said at least one cell of the microorganism.

19. The microorganism of claim 13, wherein said microorganism is a fungus.

20. The fungus of claim 19, wherein said fungus is Aspergillus niger or Rhizopus oryza.

21. The fungus of claim 19, wherein said fungus is a yeast.

22. The microorganism of claim 13, wherein said microorganism is a bacterium.

23. The bacterium of claim 22, wherein said bacterium is E. coli.

24. The microorganism of claim 13, wherein said chitin is secreted from cells of said microorganism.

25. A microorganism that has been modified to produce chitin in a vacuole of at least one cell of the microorganism, said modification comprising introduction of:
(a) (i) a heterologous polynucleotide comprising the nucleotide sequence set forth in SEQ ID NO: 7, 9, 11, or 13;
(ii) a heterologous polynucleotide having at least 90% sequence identity to the nucleotide sequence set forth in SEQ ID NOS: 7, 9, 11, or 13;
(iii) a heterologous polynucleotide that hybridizes to the complement of the polynucleotides of (a)(i) or (a)(ii) under high stringency conditions;
(iv) a heterologous polynucleotide encoding the polypeptide set forth in SEQ ID NO: 8, 10, 12, or 14; or,
(v) a heterologous polynucleotide encoding a polypeptide having at least 90% sequence identity to SEQ ID NO: 8, 10, 12, or 14;
wherein said polynucleotide of (a)(i), (a)(ii), (a)(iii), (a)(iv), and (a)(v) encodes a vacuole-targeted GFA polypeptide;
(b) (i) a heterologous polynucleotide comprising the nucleotide sequence set forth in SEQ ID NO: 1, 3, 5, 19, 21, 23, 25, 27, 29, 31, 33, or 43;
(ii) a heterologous polynucleotide having at least 90% sequence identity to the nucleotide sequence set forth in SEQ ID NOS: 1, 3, 5, 19, 21, 23, 25, 27, 29, 31, 33, or 43;
(iii) a heterologous polynucleotide that hybridizes to the complement of the polynucleotide of (b)(i) or (b)(ii) under high stringency conditions;
(iv) a heterologous polynucleotide encoding the polypeptide set forth in SEQ ID NO: 2, 4, 6, 20, 22, 24, 26, 28, 30, 32, 34 or 44; or,
(v) a heterologous polynucleotide encoding a polypeptide having at least 90% sequence identity to SEQ ID NO: 2, 4, 6, 20, 22, 24, 26, 28, 30, 32, 34 or 44;
wherein said polynucleotide of (b)(i), (b)(ii), (b)(iii), (b)(iv), and (b)(v) encodes a vacuole-targeted chitin synthase polypeptide; and,
(c) (i) a heterologous polynucleotide comprising the nucleotide sequence set forth in SEQ ID NO: 35, 37, 39, or 41;
(ii) a heterologous polynucleotide having at least 90% sequence identity to the nucleotide sequence set forth in SEQ ID NOS: 35, 37, 39, or 41;
(iii) a heterologous polynucleotide that hybridizes to the complement of the polynucleotide of (c)(i) or (c)(ii) under high stringency conditions;
(iv) a heterologous polynucleotide encoding the polypeptide set forth in SEQ ID NO: 36, 38, 40, or 42; or,
(v) a heterologous polynucleotide encoding a polypeptide having at least 90% sequence identity to SEQ ID NO: 36, 38, 40, or 42;
wherein said polynucleotide of (c)(i), (c)(ii), (c)(iii), (c)(iv), and (c)(v) encodes a vacuole-targeted chitin deacetylase polypeptide.

26. The microorganism of claim 25, wherein said polynucleotide of (a), (b), and (c) comprises a vacuole targeting signal.

27. The microorganism of claim 25, wherein said polynucleotide of (a), (b), and (c) encodes a fusion protein that is localized to said vacuole.

28. The microorganism of claim 25, wherein said microorganism is a fungus.

29. The fungus of claim 25, wherein said polynucleotide of (a) encodes a polypeptide with a lower Kₘ for a substrate compared to a native fungal GFA.

30. The fungus of claim 29, wherein said Kₘ is about 30 μM to about 50 μM.

31. The fungus of claim 28, wherein said polynucleotide of (b) encodes a polypeptide with a lower Kₘ for a substrate compared to a native fungal chitin synthase.

32. The fungus of claim 28, wherein said polynucleotide of (c) encodes a polypeptide with a lower Kₘ for a substrate compared to a native fungal chitin deacetylase.

33. The fungus of claim 28, wherein said fungus is Aspergillus niger or Rhizopus oryza.

34. The fungus of claim 28, wherein said fungus is a yeast.

35. The microorganism of claim 25, wherein said microorganism is a bacterium.

36. The bacterium of claim 35, wherein said bacterium is E. coli.

37. The microorganism of claim 25, wherein said polynucleotide of (b) encodes a polypeptide with increased processivity compared to a native chitin synthase.

38. The microorganism of claim 25, wherein said polynucleotide of (b) encodes a polypeptide with increased reaction velocity compared to a native chitin synthase.

39. A microorganism comprising a heterologous polynucleotide encoding a chitin synthase enzyme, wherein said chitin synthase has a substrate preference for UDP-glucosamine.

40. The microorganism of claim 39, further comprising at least one heterologous polynucleotide sequence selected from the group consisting of:
(a) (i) a polynucleotide comprising the nucleotide sequence set forth in SEQ ID NO: 7, 9, 11, or 13;
(ii) a polynucleotide having at least 90% sequence identity to the nucleotide sequence set forth in SEQ ID NOS:7, 9, 11, or 13;
(iii) a polynucleotide that hybridizes to the complement of the polynucleotides of (a)(i) or (a)(ii) under high stringency conditions;
(iv) a polynucleotide encoding the polypeptide set forth in SEQ ID NO: 8, 10, 12, or 14; or,
(v) a polynucleotide encoding a polypeptide having at least 90% sequence identity to SEQ ID NO: 8, 10, 12, or 14; and,
(b) (i) a polynucleotide comprising the nucleotide sequence set forth in SEQ ID NO: 1, 3, 5, 19, 21, 23, 25, 27, 29, 31, 33, or 43;
(ii) a polynucleotide having at least 90% sequence identity to the nucleotide sequence set forth in SEQ ID NOS: 1, 3, 5, 19, 21, 23, 25, 27, 29, 31, 33, or 43;
(iii) a polynucleotide that hybridizes to the complement of the polynucleotide of (b)(i) under high stringency conditions;
(iv) a polynucleotide encoding the polypeptide set forth in SEQ ID NO: 2, 4, 6, 20, 22, 24, 26, 28, 30, 32, 34 or 44; or,
(v) a polynucleotide encoding a polypeptide having at least 90% sequence identity to SEQ ID NO: 2, 4, 6, 20, 22, 24, 26, 28, 30, 32, 34 or 44.

41. The microorganism of claim 40, wherein said polynucleotide of (a)(i), (a)(ii), (a)(iii), (a)(iv) and (a)(v) encodes a polypeptide with increased GFA activity compared to a native GFA.

42. The microorganism of claim 40, wherein said polynucleotide of (b)(i), (b)(ii), (b)(iii), (b)(iv) and (b)(v) encodes a polypeptide with increased chitin synthase activity compared to a native chitin synthase.

43. The microorganism of claim 39, wherein said microorganism is a fungus.

44. The fungus of claim 43, wherein said fungus is Aspergillus niger or Rhizopus oryza.

45. The fungus of claim 43, wherein said fungus is a yeast.

46. The microorganism of claim 39, wherein said microorganism is a bacterium.

47. The bacterium of claim 46, wherein said bacterium is E. coli.

48. A method for producing chitin, comprising:
a) culturing the microorganism of claim 1 under conditions which result in chitin production; and,
b) isolating said chitin.

49. A method for producing chitin, comprising:
a) culturing the microorganism of claim 13 under conditions which result in chitin production; and,
b) isolating said chitin.

50. A method for producing chitin, comprising:
a) culturing the microorganism of claim 25 under conditions which result in chitin production; and,
b) isolating said chitin.

51. A method for producing chitosan, comprising:
a) culturing the microorganism of claim 1 under conditions which result in chitin production;
b) isolating said chitin; and,
c) converting said chitin to chitosan by a chemical process.

52. The method of claim 51, wherein said chemical process comprises strong alkaline treatment.

53. A method for producing chitosan, comprising:
a) culturing the microorganism of claim 13 under conditions which result in chitin production;
b) isolating said chitin; and,
c) converting said chitin to chitosan by a chemical process.

54. The method of claim 53, wherein said chemical process comprises strong alkaline treatment.

55. A method for producing chitosan, comprising:
a) culturing the microorganism of claim 25 under conditions which result in chitin production;
b) isolating said chitin; and,
c) converting said chitin to chitosan by a chemical process.

56. The method of claim 55, wherein said chemical process comprises strong alkaline treatment.

57. A method for producing chitosan, comprising:
a) culturing the microorganism of claim 39 under conditions which result in chitosan production; and,
b) isolating said chitosan.

58. A plant that has been modified to produce chitin, said modification comprising introduction of:
(a) (i) a polynucleotide comprising the nucleotide sequence set forth in SEQ ID NO: 7, 9,11, or 13;
(ii) a polynucleotide having at least 90% sequence identity to the nucleotide sequence set forth in SEQ ID NOS: 7, 9, 11, or 13;
(iii) a polynucleotide that hybridizes to the complement of the polynucleotides of (a)(i) or (a)(ii) under high stringency conditions;
(iv) a polynucleotide encoding the polypeptide set forth in SEQ ID NO: 8, 10, 12, or 14; or,
(v) a polynucleotide encoding a polypeptide having at least 90% sequence identity to SEQ ID NO: 8, 10, 12, or 14;
(b) (i) a polynucleotide comprising the nucleotide sequence set forth in SEQ ID NO: 1,3,5, 19,21,23,25,27,29,31,33, or 43;
(ii) a polynucleotide having at least 90% sequence identity to the nucleotide sequence set forth in SEQ ID NOS: 1, 3, 5, 19, 21, 23, 25, 27, 29, 31, 33, or 43;
(iii) a polynucleotide that hybridizes to the complement of the polynucleotide of (b)(i) or (b)(ii) under high stringency conditions;
(iv) a polynucleotide encoding the polypeptide set forth in SEQ ID NO: 2, 4, 6, 20, 22, 24, 26, 28, 30, 32, 34 or 44; or,
(v) a polynucleotide encoding a polypeptide having at least 90% sequence identity to SEQ ID NO: 2, 4, 6, 20, 22, 24, 26, 28, 30, 32, 34 or 44;
(c) (i) a polynucleotide comprising the nucleotide sequence set forth in SEQ ID NO: 35, 37, 39, or 41;
(ii) a polynucleotide having at least 90% sequence identity to the nucleotide sequence set forth in SEQ ID NOS: 35, 37, 39, or 41;
(iii) a polynucleotide that hybridizes to the complement of the polynucleotide of (c)(i) under high stringency conditions;
(iv) a polynucleotide encoding the polypeptide set forth in SEQ ID NO: 36, 38, 40, or 42; or,
(v) a polynucleotide encoding a polypeptide having at least 90% sequence identity to SEQ ID NO: 36, 38, 40, or 42.

59. The plant of claim 58, wherein said plant is selected from the group consisting of maize, sorghum, wheat, sunflower, tomato, crucifers, peppers, potato, cotton, rice, soybean, sugarbeet, sugarcane, tobacco, barley, and oilseed rape.

60. A transformed seed of the plant of claim 59.

61. A method of producing chitosan in vitro, comprising:
a) expressing chitin synthase in a first microorganism;
b) isolating and purifying said synthase;
c) expressing chitin deacetylase in a second microorganism;
d) isolating and purifying said chitin deacetylase;
e) linking said chitin synthase and said chitin deacetylase to a resin;
f) contacting said resin with permeabilized cells producing UDP-Gln or UDP-NAc-Gln and,
g) isolating said chitosan.


SEQUENCE LISTING (44 sequences)

SEQ ID NO: 1 - DNA, 4387 bp, Saccharomyces cerevisiae
  CDS (458)...(3853)
tgaaattacgtacatatacataagtagaagataattcgttgcgcatatgctacaagaacccttggtgaaacgaaatttgatacaagtaaatacatgcaggaaacatacattacctctgaaacaaagccgagacggggcccagcagtctttttttagaaatcgcgtggcttggtaacgcgataatgacgcgacacagccattagtgtgaaatttgattttcttggccaaactaggtataatatttgttacaaattattgattttaatatatatctcgggttcattttttacgtcggtactccaaaggatcaaaacacttacattttgaggcctaccggaccttgcagtactgcttgtttaaatacgcagtatacatttcttcttcttcttctctcttctttccttcctcgaagagtcactaaattaatactgggaagccaaaccaaaaaaactatacaatgagtgatcaaaataatcgatcgagaaatgaatatcactcaaaccggaagaatgaaccttcctatgaactccaaaatgcacatagcgggctatttcactcttctaatgaagaattaacaaacaggaaccaaagatataccaatcaaaatgccagcatgggttcattcactccagtccaatctttgcaatttccagaacaatctcagcaaacaaatatgctttataacggtgacgatggcaataataatactatcaatgataacgaacgagacatatatggaggttttgtcaaccaccatcgccagcgtcccccaccagcaactgcagaatacaatgacgtttttaatacgaatagtcaacagctaccgtcggaacatcaatacaataacgtaccttcatatccacttccttcgataaatgtgattcaaaccactccagaactcatacataacggctcacagactatggccacccccatcgaaaggcccttctttaacgaaaacgactactattataataacaggaactctaggacgtcaccgagtattgcttctagtagcgatggttatgcagatcaggaagctaggcccattttggagcaacccaacaataacatgaatagcggtaatattcctcaataccatgaccaaccttttggatacaacaatggttaccatggcctacaggcaaaagattactatgacgatccggagggtggttatattgatcagagaggagatgactatcagattaattcatatttgggtagaaacggtgaaatggttgatccttacgattatgaaaacagtttaagacatatgactcctatggagcgtagagaatatcttcatgatgatagcagacccgtaaacgatggaaaagaagaattagacagtgtgaaaagcggttactctcatagagacttgggggaatatgacaaggatgatttttcaagggatgacgagtacgatgatctcaacactattgataaattacagtttcaagctaatggtgtacctgcatcatcctcggtgtcttctatcggatctaaagaatccgacataatagtaagcaatgataacttaaccgcaaatagagcactaaagagaagcggtactgaaattaggaaattcaaactttggaatggtaattttgttttcgattctccaatcagtaagacgctattggaccaatacgctactacaacagaaaatgcaaacactttaccaaatgagtttaagtttatgagatatcaagcagttacttgcgaacctaatcaacttgcagagaagaatttcacggtgaggcagttgaagtatttaactccaagggaaacggaattgatgctagtagtcacaatgtataatgaagaccatatcctgttaggaagaactttgaaaggtattatggacaatgtcaaatatatggtgaaaaaaaaaaattcaagcacttgggggccggatgcatggaaaaagattgtcgtttgtatcatttcagatggtagatccaaaattaatgaacgctcgctagcattactaagttcgttaggttgttaccaggacgggtttgctaaggatgaaattaatgaaaaaaaagtggcaatgcatgtctacgaacatacgacaatgatcaacatcacaaatatttcggaatcagaggtttcattagaatgcaatcaaggtaccgttccaatacaacttttgttttgtttgaaagagcaaaatcagaaaaaaattaactcacatagatgggcatttgaaggctttgcagaattactgcgtcccaatatcgttacattgttagatgctggtaccatgccaggtaaagattctatttaccagttatggagagagttcaggaatccaaatgttggtggcgcatgtggtgaaataagaactgatttgggtaagagatttgtaaagcttttgaatcctttagttgcatcacagaatttcgaatacaaaatgtccaatattttagacaaaacaaccgagtctaactttggatttattactgttctaccgggggcattctctgcgtataggtttgaagctgtgagaggccaaccattacagaagtacttttatggtgaaattatggaaaatgaaggttttcattttttttcttccaatatgtatcttgctgaagatcgtattttatgctttgaagtggtcacaaaaaaaaattgtaattggattttgaaatactgcagaagttcttatgcttcaacagatgtaccggagagggtccctgaatttattcttcagaggaggcgttggttgaatggttcattttttgctagtgtatattccttttgtcatttttacagagtctggagcagtggtcataatattggtagaaaactccttttgacggttgaatttttttaccttttcttcaatacattgatttcatggttttcattgagttcatttttcctattctttagaattctcactgtttctattgcactggcataccattcagcatttaatgtgttgtccgtcatattcctgtggctttatgggatttgtaccttatcaacattcatactgtcattgggtaataaacctaaaagtactgagaaattttatgttctaacttgcgtcatttttgcggtgatgatgatttacatgatattctgcagtatattcatgagtgtcaaatccttccaaaatatattgaaaaacgataccatcagctttgagggtttgattaccacagaagctttcagggatattgttatctctctgggctccacttattgtttgtacctaatcagttcaattatctatttgcagccatggcatatgttgacaagttttattcagtatattttattgagtccttcttacatcaatgttttgaatatctatgcattttgtaatgtccacgacttatcatggggtacaaagggtgcaatggcaaatccgctgggtaagattaatactacagaagatggtacgttcaaaatggaagttctggtctctagttcagagattcaagcaaactacgataaatatttgaaagttttaaatgacttcgatccaaaatcagaatctcggcctactgagccatcttatgatgaaaaaaagactggctattatgcaaacgttagatctctcgtgattatcttttgggtcatcacaaatttcatcatcgttgctgttgtcttagaaaccggtgggattgcagattatattgctatgaaatccatatcaactgatgacactttagaaactgcaaagaaggcggaaattcccttaatgaccagtaaggcctcaatttattttaatgtaattttatggttagttgcattatcggcattaataaggttcattggttgctcaatatacatgatagtaaggttttttaaaaaggttacatttcgctaagtagattgattacccctttgttataaataagtatatgtgtttactatcatataataattaattagttgtaagtatggtatttttcaaagaaacatttagtattatttttaaagggtgcctttaccataaaaccaaaaaaaaatggcttctgagagaaaaacataaataattgataaaactgaaactcacaacaccaactcttccgctctaaacacccaaagctcaaaaaacgacatgtgcacatctatatccataaactttcaaaaaagtggttcattcgcaaattcatgaagtattatttaatacacagtaatctggcatttcatactatcattgctcaaattatccgccggacccttgaaactgtggcgctttttaagacgcatctccaaaaaaagaaaaagaacaaaagaaaaaaagctgcaaatatatatacaaaacagatttcacgtttattgcttgggagtaaggtaacgatccggaagccttgaagaaatttgaatgtatccaactaactattgaaaatcattcgtg

SEQ ID NO: 2 - protein, 1131 aa, Saccharomyces cerevisiae
MSDQNNRSRNEYHSNRKNEPSYELQNAHSGLFHSSNEELTNRNQRYTNQNASMGSFTPVQSLQFPEQSQQTNMLYNGDDGNNNTINDNERDIYGGFVNHHRQRPPPATAEYNDVFNTNSQQLPSEHQYNNVPSYPLPSINVIQTTPELIHNGSQTMATPIERPFFNENDYYYNNRNSRTSPSIASSSDGYADQEARPILEQPNNNMNSGNIPQYHDQPFGYNNGYHGLQAKDYYDDPEGGYIDQRGDDYQINSYLGRNGEMVDPYDYENSLRHMTPMERREYLHDDSRPVNDGKEELDSVKSGYSHRDLGEYDKDDFSRDDEYDDLNTIDKLQFQANGVPASSSVSSIGSKESDIIVSNDNLTANRALKRSGTEIRKFKLWNGNFVFDSPISKTLLDQYATTTENANTLPNEFKFMRYQAVTCEPNQLAEKNFTVRQLKYLTPRETELMLVVTMYNEDHILLGRTLKGIMDNVKYMVKKKNSSTWGPDAWKKIVVCIISDGRSKINERSLALLSSLGCYQDGFAKDEINEKKVAMHVYEHTTMINITNISESEVSLECNQGTVPIQLLFCLKEQNQKKINSHRWAFEGFAELLRPNIVTLLDAGTMPGKDSIYQLWREFRNPNVGGACGEIRTDLGKRFVKLLNPLVASQNFEYKMSNILDKTTESNFGFITVLPGAFSAYRFEAVRGQPLQKYFYGEIMENEGFHFFSSNMYLAEDRILCFEVVTKKNCNWILKYCRSSYASTDVPERVPEFILQRRRWLNGSFFASVYSFCHFYRVWSSGHNIGRKLLLTVEFFYLFFNTLISWFSLSSFFLFFRILTVSIALAYHSAFNVLSVIFLWLYGICTLSTFILSLGNKPKSTEKFYVLTCVIFAVMMIYMIFCSIFMSVKSFQNILKNDTISFEGLITTEAFRDIVISLGSTYCLYLISSIIYLQPWHMLTSFIQYILLSPSYINVLNIYAFCNVHDLSWGTKGAMANPLGKINTTEDGTFKMEVLVSSSEIQANYDKYLKVLNDFDPKSESRPTEPSYDEKKTGYYANVRSLVIIFWVITNFIIVAVVLETGGIADYIAMKSISTDDTLETAKKAEIPLMTSKASIYFNVILWLVALSALIRFIGCSIYMIVRFFKKVTFR

SEQ ID NO: 3 - DNA, 4123 bp, Saccharomyces cerevisiae
cttttctacgtttgctgttttttctgtttcccttgccttatacgaacatacgtttctaatgccgattataaacacaatagacacaattatactattttctcacttttctcttcgggagatcgctattgattattattcccgtttatttccttcaatttagggtctgaaaagaagatagtagggttagttttttggggtttaaaaccctagtataaacccctatactatttgtttatatatttcctatttgagtatgatagcatcgatattgaaatttacagaacttcaatttagtttcaattgggaattcgaggactctcccttgggatggccccagtctatttaactatcttccataatattactgttgatttcgtgactcagaagagaaaacagaggaagttacatatagacccaaataaaaaccaaagaaccacatatagaaatgacgagaaacccgtttatggtggaaccttcgaatggctctcctaatagacgtggtgcttcaaacctctccaaattttacgcaaacgctaacagcaactctcggtgggctaatcccagtgaggagagtttggaggatagctatgaccaatctaacgttttccaaggccttccggcatctccttcgagagctgcactaagatactccccagaccgtcgccatagaactcaattttaccgcgatagtgcccataactctccagttgctccgaacaggtatgctgctaatctacaagagtctcccaaaagagcaggcgaggctgtcatacatctaagtgaggggagtaacctttacccccgcgataatgcagatctaccggtagacccctaccatctatcaccccagcaacagcccagtaacaatctgtttggaagtggcagattgtattctcaaagctcgaaatacacgatgtctactacttccacaacggctccctctctggcagaagcagacgatgaaaaggaaaaatacctcacttcgactacttcctatgatgatcagtctacaattttctctgcagacactttcaatgaaacaaaatttgaactgaaccatccaacaagacagcagtatgtaagacgtgccaattctgagagtaagagaagaatggtctcagacttgcctcccccaagcaagaagaaggcactattgaaactagacaacccgataccaaaaggtctgttggatactttgcctcgtaggaactcccctgagtttacggaaatgagatatacagcctgcactgtggaacctgacgattttttgagagaaggttacactttgagattcgcagagatgaacagagaatgtcaaattgccatttgtattaccatgtacaatgaagataaatattcattggcaagaaccatccactccattatgaagaatgttgctcatctttgtaagcgtgaaaaatctcatgtttggggccccaatggctggaagaaagtctctgtaattctgattagtgacggtagagcaaaagtcaaccaagggtctctcgactatttagctgctttgggtgtttatcaagaagatatggccaaggcgtctgtgaatggtgatccggtaaaagcgcacatttttgaattgacaactcaggtctctatcaacgccgatctggattatgtttcaaaggacattgttcctgtgcaattggttttttgtctaaaagaagaaaataagaaaaagatcaattcccatcgttggctattcaacgcgttttgtcccgttttgcaacctactgtagttactttggttgatgtcggtacacgtttaaacaatacagcaatttacaggttatggaaggtttttgatatggattcaaatgtggctggtgcggctggtcagattaagactatgaaggggaagtggggactgaaacttttcaatccattagttgcatcgcaaaattttgaatataagatctcgaatattttagataaaccattagaaagtgtttttggttatatctctgttcttcctggtgccctgtccgcatataggtatagagccttaaaaaatcatgaagatggcactggccctctcagatcgtatttccttggtgaaactcaagaaggcagagaccatgatgttttcactgcaaatatgtacttggctgaagatagaattctttgttgggaattggtcgccaagcgagatgcaaaatgggttctaaaatatgttaaggaagccactggtgaaacggatgttcccgaagacgtttctgaatttatttctcaaagaagacgttggttaaatggtgcaatgtttgccgcaatttatgctcaattgcatttctaccaaatttggaaaactaaacattctgttgtacgcaagttctttcttcatgttgaattcctttatcaatttattcagatgcttttttcctggttttctattgcaaatttcgttcttaccttttattatttagcaggatcaatgaatttagttattaaacatggtgaggccttattcattttttttaaatacctgatcttttgtgacttggcaagtttattcattatttccatgggtaatagaccccagggcgcgaaacatttattcattacctccatggttatactgtctatatgtgccacatattctctaatttgtgggtttgtttttgctttcaagtcgttagcttctggaacggaatcccacaaaatatttgtcgacatcgttatctcattgctctccacctatggcctatactttttctcatcactgatgtacctagatccttggcacatgtttacatcatccatacaatactttttgacacttcccgcctttacgtgtactttacagatttttgccttctgtaatacacacgacgtttcctggggtactaaaggttccacacaggagtccaagcaattgtccaaggccattgtcgttcaaggtccagatgggaaacagattgtggaaacagattggcctcaggaagttgataagaagtttttggaaataaaaagtcgtttgaaagaaccagaatttgaagaatcaagcggcaatgaaaaacaatccaagaatgattattatagagatataagaaccagaattgtgatgatttggatgctatcaaatctaatactgatcatgtctataattcaagtctttacaccacaagatactgacaatggttatttgatattcattttatggtctgtggccgctttagctgcctttagggtggttggttccatggcctttttgttcatgaaatacttgcgtataatagtgagttacagaaataaagttgaaggtagcggctcatgggaagtctctaaattagacttaccaaatgttttccacaaaaagggctaatgccagtatttttcagctaatttctcgtcattccctcttttttctcattctgttttagaatgtacaggttatgacaaccattaccttctaggatattttgacatttctctcttagactttttatatataatatacatgtttcaataccaaatataatgtagcataacataatctatctttttactcatagacctttactttcctctctaaaagccgtgtcgcagtaactggtgacgctaaagttctccggcgtttttttatgtatgagctgggcatcaagaggcttttgagattttccaagtagtaactcatctttctgagtgtgctatcaaatacatactaaggagaataaactcttgttattacgtattcttcatccttatgggtagagagcgcactgttttagtacattttctagacgtcgaaacgtagagcaattgtcgataaaacaaaaaaaaagtaagaagatatatgaataggacgtgtcgctagaactagtaagtatatgatggagatataataagtgaattattcgatatttaatgaacgttctcatttatttggaagaaatgtttatcacgtgatggagaaccaatgagcggcgagtaactacgcgaggaacccggaccgcaataacgattaaagaaggcccggaagggagatgcttaaatgattatcactcagttaaaaaagacaaataagaaactattgagactgaaccgttttggttaatttcaggtggaaacaattgaagacgagcagtaaacattattttatttagtagtc

SEQ ID NO: 4 - protein, 963 aa, Saccharomyces cerevisiae
MTRNPFMVEPSNGSPNRRGASNLSKFYANANSNSRWANPSEESLEDSYDQSNVFQGLPASPSRAALRYSPDRRHRTQFYRDSAHNSPVAPNRYAANLQESPKRAGEAVIHLSEGSNLYPRDNADLPVDPYHLSPQQQPSNNLFGSGRLYSQSSKYTMSTTSTTAPSLAEADDEKEKYLTSTTSYDDQSTIFSADTFNETKFELNHPTRQQYVRRANSESKRRMVSDLPPPSKKKALLKLDNPIPKGLLDTLPRRNSPEFTEMRYTACTVEPDDFLREGYTLRFAEMNRECQIAICITMYNEDKYSLARTIHSIMKNVAHLCKREKSHVWGPNGWKKVSVILISDGRAKVNQGSLDYLAALGVYQEDMAKASVNGDPVKAHIFELTTQVSINADLDYVSKDIVPVQLVFCLKEENKKKINSHRWLFNAFCPVLQPTVVTLVDVGTRLNNTAIYRLWKVFDMDSNVAGAAGQIKTMKGKWGLKLFNPLVASQNFEYKISNILDKPLESVFGYISVLPGALSAYRYRALKNHEDGTGPLRSYFLGETQEGRDHDVFTANMYLAEDRILCWELVAKRDAKWVLKYVKEATGETDVPEDVSEFISQRRRWLNGAMFAAIYAQLHFYQIWKTKHSVVRKFFLHVEFLYQFIQMLFSWFSIANFVLTFYYLAGSMNLVIKHGEALFIFFKYLIFCDLASLFIISMGNRPQGAKHLFITSMVILSICATYSLICGFVFAFKSLASGTESHKIFVDIVISLLSTYGLYFFSSLMYLDPWHMFTSSIQYFLTLPAFTCTLQIFAFCNTHDVSWGTKGSTQESKQLSKAIVVQGPDGKQIVETDWPQEVDKKFLEIKSRLKEPEFEESSGNEKQSKNDYYRDIRTRIVMIWMLSNLILIMSIIQVFTPQDTDNGYLIFILWSVAALAAFRVVGSMAFLFMKYLRIIVSYRNKVEGSGSWEVSKLDLPNVFHKKG

SEQ ID NO: 5 - DNA, 5176 bp, Saccharomyces cerevisiae
taattgccttagccacgtaatatgtgcctttaaagatagagaaatatttatttattaagttttagttctaaggtgtgttaaaatacctctcaaataaataagttacacacaaccatatatcaacttgtaagtatcacagtaaaaatattttcatactgtctatgcaacgaaggagtcactttcctccttccgattgagaatatcttccctttcaaattccctccatgtcctcattttaatctttgagtgatcaaattcaccttcattctcgtcttgtgcctttttattacctcccgcaatagttctcgtatcaccccatgagaagtcatcaaatttccagtacgcatatgaaggtagtacgaaattccaaataggcaaagcacaaatatatacgcacatccaccataggtacgaccatctcgtagcagttataacaacaattaagccgggcagaccaagaataattgccagtaaaactaaagtgattacgggtgtaggttttgatacaatggcaaaaataatgacataaatagtaaagcaaatggctaacggcagtaccatagtaccaatcaattcaataccaatcacaaattgcatggaaaaacagaaagtgccacataagtctctgattagaactaattcaaaaaggttatgtaccgtagaattaatccatcttcgacgctgggaaagtaagactttgaatttatcaggggcaatagttttacaagcagcttttggaacaaatacttgctttctcttagggaaagtctttaacattaatgaagataaaaatctatcttcaccaagtaataataagttcttcttatgcaaagtgtttgtaacattatccgaatatctttcaacaatatctggatttgccaatacaggtacccaataaccatctgaacctttaggagattttatacgatacattgagaaacatcccggcaaacaagttaccgaaccgaagacagattcaaaagctttagcctgatgatgcgaaatatagtactcaaacacttgaattgcagttacccaagattgtgccttattagcgatcttggtctcaccacaaagacccataatcaaaggatctttaaccatttcagcgaccatatgagttaaagcatcgggaaagactttagtatcagcatcaaccataagtaccgtttcgtagaagtctgccattagccccgtaatctgccaaatattttttaaaagctgaaattccaattgagtcattctttcatcaaatgttattttttctaaaaaggacatcagaataatttgagaatcacgcttacctctgttaccgggtttggcggccccctgctctgcaggagtaccgcacttcacaattgtaatgattgggacacgttgttgattttctggtggaattgtagaatcgtcatatttgtaaaaacccgcatatatcttggccatattgtgtcttttagagcctgatgccactgccacataggagtaaggtttaacttcatcaggtggggtgacaaagtcgtccatcattcctaacgctatctctggagtagtcttatcgttgcccgagcccttaattaaaccatcacaaacaaccatcagtagtttatgggaatttggataatctgtggtagaaagagagtctaaagtggttcttaaaccctcttcatcctcagaataacaagtaacaaaacagatagtatgaatcaatgggaacccgtatggcataaaatccagtggtggttgttgaacgatatcgggatgtataatcgtcgaatcaagactctgaatcagcgatgatcctggtacaggagaggaagtcgctttattccaaaacatcgaggatgtaggcaacaaagtagatggattacgggaatgtacagctttgttttcattcgagagtttccaagcattttgagtggtcattgttgtcattcctctgtatgaaccagaagattgtagtgaactgctcatggaggtgtctagatctatcacagattcgttaaattgaaacattttggagctgtgttttttcagcaagtcaaaggttgaagcacgcttgtgtcccaacgacttttttgagtatttctttggcctcaaatgaggatctacttcctttagaggagcttttgtttgaatattattagaccaatcctcgatatcgtttgtgtgtttatccattgttttattgtccacgatatatgcaccttgtttcctagctacagtccaacggaagtagcaggcaattatgaatttaattatcaccactgaaagaataaataccagagaaacatacaaaacgacatcagaggcaatacaaccgacggttttggagtctacttcaccaactttaataatttcgctcaaacatctcgcaatttttctttcatgcccatttgacaaaaccaacgaaagatcataaccttgtaaatttgaagtcttcaagtcatcgaatacaacgggatagtcaacgtcatccttttccaaccaatcaagaagatctaaatccaaaacgtcgccattataaacaatcaagtttctagaagagttctttataccatcccaagtgaagtaaacatcagctttcgactttaaaccgtaaaatgcgtccctatcttctttagacgtatgacagttccatcctgcgtaattttcaactgtgaagtttggcttcgaagagccatcttgattctttaacttacaaggaaaataccatgctaaattattatcatcgtcatggggaatggaagaattactctttggagttataaggttatgacagttaccattcacattttgaaacaagaacgaagcatctttaccagcatctgaccagggcccataaagggtgtctgaatctacttcaacgtcttgtataccggaacgcgaggaagtatccaattcataagccttaccgttaattacgacaaattctgttgatacttcgttgtttttcaaacgtagtttcgaactactacaaacggttttagtgaaaccaaaagtcaggaaagccacaatcgcaccaatgtacaagatgacagaaattaaagcaaccttttctctccacgccatttgtctttccttctttggcatcccgcagaaagcaagaattggagcaggtgcccaaaacgtaatgaaataacagtacatctgccaaaatgacaatgtatcattcgtttcctttcttaatggtttgatagacttatttatgggctgcgtggagctttcttcgaaattgtccacatcaccatctttatcaaattcgtcttccgcaacaccttcatcacttattttaacggaagccttcttgtcagtagtattcatatcttgtaaaagatagctatccgtttcgcggccactaaatttgcttctcactgagcctttggagcgcagggagccactccgacgagttgcatttgggtttacaccggtacttgatggtaaaacatccaggtgattcatctgctcctgcgttttctgcgcataataaaaatgtggattatcaggattgttcagtcggcttctttcgggccgcactaaagagccttgtctatgaggtgctcctgagccgacactgtgtcttgacctaagtagagactcttcatcttgattaaggttcagatagtagtcatcaggatcatctccattcaagccggtcattctaatttctttcctgcggatagtctaaacaggacctttgaagaaaatgggacctacaccaaaaatactcgagaactttgcaaatcggtaaacgtttataaccgtctaccaatgtgattcttctagcaggatttactacaagtcgaaatgttggtactgaatgtaaatctgaaacacaggaaagaatatttttgatttctatttttatgctcttattttcatttttgattttttttttcgaggtctgcgccagcgccaataacgcgttttatccaaataggcagccaaagagggtcacctatgcaggtgctgaatgcaatacaactgcgggctaactgtccatcgacgctattgaatatgcttcaagaaatatccagtctcatgcacatgtcaaatatgtgggtgagggcgcaaatacaacgcacgattatatatacatttgtggctacgtagtacgtgcggagataggcaggtatttgttcaaggatgtaaataagaactacaatgcttagttctcgaatcctagaatgtgagtaaatctcacaagcaccgcttctataaatttgtctagggtaatttgtaatcttttcccatgcggattctcttaaatacggtacagccgtcgctgctgcggtgtgattctggtttcatagatctggaaaaagaaagcgagacacaatacaagagctttgtgacacagcgtacggagagaataagataaagaacagttcctggcgtttttaattgaacttttgtactcgtctctttattctcacgaatttttttaccgccgttttcatttccattgcaaaggagggaaaatataaatgtttttatagaggcagtatatctttaatttcatagcatatgcagtttagaaaaaagacgtaaattcgttgccctagtcacgctggttacataaattggtcgttgaattgacttttaagacatccagttggtgtgtttgctccaaatagtgctgacgcatcttgccaattctcgtcaatttagcacggaatgcttgttacttattcgataacgcttttaccttgattgcatgtagtcactatcctcaaaccttttctaaggttgccctattggcgacagaaaagcaggacgagaaatctaaataaccaacgtgtggtttattaactatgacggttggtttgcttgccgccccaagttatagaagactgcccttcatcccggggtagctatcatagaatctgtcgatagttacaattttgggtggggtgttttcaaaaatggacggatatgccggtctttgaaacttcgatgatgtagcctattgttttaattttctgcttgatcctttcgctctttttagggataaaaaagaaaacctaacaaagtatgaatttcgagaagtcccatcgatagtagtcaaacaattgctaaagagggctacggatcgtctacaataccttcgtaaaacagcagcttacaggatcttttataatatcaccatagaaaggggaatttgggaagcgagagtaaa

SEQ ID NO: 6 - protein, 1165 aa, Saccharomyces cerevisiae
MTGLNGDDPDDYYLNLNQDEESLLRSRHSVGSGAPHRQGSLVRPERSRLNNPDNPHFYYAQKTQEQMNHLDVLPSSTGVNPNATRRSGSLRSKGSVRSKFSGRETDSYLLQDMNTTDKKASVKISDEGVAEDEFDKDGDVDNFEESSTQPINKSIKPLRKETNDTLSFWQMYCYFITFWAPAPILAFCGMPKKERQMAWREKVALISVILYIGAIVAFLTFGFTKTVCSSSKLRLKNNEVSTEFVVINGKAYELDTSSRSGIQDVEVDSDTLYGPWSDAGKDASFLFQNVNGNCHNLITPKSNSSIPHDDDNNLAWYFPCKLKNQDGSSKPNFTVENYAGWNCHTSKEDRDAFYGLKSKADVYFTWDGIKNSSRNLIVYNGDVLDLDLLDWLEKDDVDYPVVFDDLKTSNLQGYDLSLVLSNGHERKIARCLSEIIKVGEVDSKTVGCIASDVVLYVSLVFILSVVIIKFIIACYFRWTVARKQGAYIVDNKTMDKHTNDIEDWSNNIQTKAPLKEVDPHLRPKKYSKKSLGHKRASTFDLLKKHSSKMFQFNESVIDLDTSMSSSLQSSGSYRGMTTMTTQNAWKLSNENKAVHSRNPSTLLPTSSMFWNKATSSPVPGSSLIQSLDSTIIHPDIVQQPPLDFMPYGFPLIHTICFVTCYSEDEEGLRTTLDSLSTTDYPNSHKLLMVVCDGLIKGSGNDKTTPEIALGMMDDFVTPPDEVKPYSYVAVASGSKRHNMAKIYAGFYKYDDSTIPPENQQRVPIITIVKCGTPAEQGAAKPGNRGKRDSQIILMSFLEKITFDERMTQLEFQLLKNIWQITGLMADFYETVLMVDADTKVFPDALTHMVAEMVKDPLIMGLCGETKIANKAQSWVTAIQVFEYYISHHQAKAFESVFGSVTCLPGCFSMYRIKSPKGSDGYWVPVLANPDIVERYSDNVTNTLHKKNLLLLGEDRFLSSLMLKTFPKRKQVFVPKAACKTIAPDKFKVLLSQRRRWINSTVHNLFELVLIRDLCGTFCFSMQFVIGIELIGTMVLPLAICFTIYVIIFAIVSKPTPVITLVLLAIILGLPGLIVVITATRWSYLWWMCVYICALPIWNFVLPSYAYWKFDDFSWGDTRTIAGGNKKAQDENEGEFDHSKIKMRTWREFEREDILNRKEESDSFVA

SEQ ID NO: 7 - DNA, 3554 bp, Saccharomyces cerevisiae
cccgctatttccaatgctcccactatttgttgatacttgcctctttcagagctatctgccttatgttgattattagggttaataaatgctatttcctcccgcaataattgtgtaatttgaacgaagaatatgaagaagatggaatggcgattttcactctaaattttaaaaattgcctctttacaatagcgaatttcctaacccttttttttttttgttgattgcctattgctcgttcacttcccatttattttctctcgaatttcaccaaaagttgatgtggataatcaatcatcgggcctattcctgcgggtaaaacgcagggcccaactcaggatagggtttaatattattttagaggacttacaagaaggaagttatatggtttaaaaattgtaacaaagttagaacacatttatttagcaggtctaatttagggctgcaactatctttttggttattcatataaaatataattttttatttatatagagaatacaagtggaatcatctttaacgccagcttgtagtgcgcattgcagaataatggaagttcaaaaattaaaagcgaaggagaagtgattgtagaaagacggatgggaggctgggggacgaagagaaagtaaaagggttaatttattcgacggtaacagatttagccaagtttcttggaaaatcaacatcaatccctttattaacagccaaccaatatgacattagttgtaatggaataatattaattagaccttgtaaacaatcaacagtttgtggaacttctaaggtttgcaggtcgattgatttagatttttgcgcccacacttcatcattttcgttacaaataataattggatggccctttcttgcagtaacttgctcaatagaggaaactactttagggaatagagagtctctggtaccaaaagcaatgattggcaagttttcgtccaccaaggccaagacaccgtgcttcaactcacctgccaaaacaccttcagaatgcatataagaaatttctttgatcttcaaagcaccttccagagcagcagcaaattggtaacctctacccaataacaatagagatttttgatcctttaattcagtcgcacagagcttttttattcttggttccagctttaatacctgcttaatttggcccgggattaacttcaagccttgaatgatttcaattcttctgtctattttcgatacacggtcatctgacagcgatagagcaaacatcactaaggcaatatactgggaagtataagcttttgtagaggcaacaccaatttcaggaccagcgttaatatgaacaccacagtgggtgacacgagagatagaagaaccaacactgttaacaattccgacagttaaggctcctctttctaaacaataatttagagccagcatggtatccgcagtttcaccactttgtgaaacaaacacgcatacatcgtctctgaagacagggcattttctgtccagaaagtcagacgctaattccacactaactgggatatctgataattcttcgaagatagcacgagtagccaaacatgaatgataagaagtaccgcatgcgatcatgatcagtctccgtgctcttctgacaactggtaaccatgcctttaaaccacccaatatcactttattattttcatagtcgattctacctctcatagtattgaaagtagattctggttgctcatagatttccttttgcataaaatggtcgtaagggcccttcatgatctgagctaactccatctctaaagtttgaatggaccttgtcattgatgcgcctacttctcttctagatctatgaatatgtaactcaccatcgtaaatatgagccaaatcgtcatcttctaaaaatagcaccttcttggtatgtttaacaacagatgccgcatccgaagaaacaaaaaattccaccggtgttggagatccatcttctgataggaaagccctggattgagaatgtctcaagttaaattcattggcggcaattggtagtaaattggcattttgggaaccagcttcaaattcacgagctttctttgggcccaagccaaatgatttgttgttagatttcaatggaatttccggttgaccagcgttttcttcgggaaattccacatccacgaagtcgacttttagttttttttcagatttgacaccaatcagtaaaggggacccttttctagtggcgataacctcattaggatagtgacaagatttacataataacccgtatgaaccttctagttctaaaagaactagcttggttaattcgtggaaatctaagtcatgcccattttgtaaatttgtattgtataaatgcaaatatagtttagcaatacactcggtatcggtatcactttcgaatttataacctttgttaattaaaagagtcttcagttctctaaaatttgtgatgataccattatgaacgaccacaaattggtcttctgggtcagatctttgagggtgacagttaacttgttctggtcgaccgtgagtagcccatctagtatgcgcaataccacaatgagagacaaaagtaacgtctctgttcggattttgcttagtaatctcctctttcaaagcactcactttaccgatttgcttatagatgaaagtagaatcagcttcgtcaccatcgatagcaataccggtggaatcatagcctctatattctaatctttgtaaaccatccactaaggtgtcgataatttctcctctggatctttccactagataattgcagtaaccaaagataccacacatttttttataaaagttgctgttgatttgctcgagaacttattgcttatttggccctgataactatataagaaaagaaatacagttattccttgtttatgctggctttttgtccactttttctcaactatataactatgatgttggaaaggacaccggttctgtaactttgcagtgaaaataagtgtgatggatgactgagaatgctttcttgtaagcgaaaagaagtacgtgttccaaaaataaagcagaaaggcgaaaagggtcgaatgtaagacactaaataaatattttaagaagaggaaaagtcgcctcagaaacgctaaaatgcatccgatttcccaaagaggaagtctaatgttttcgatttgtgaaaaaaagataaaaatcgaagaaaatgtagggagccgcgcgttacccggattgatatttgagtgatcgacggcgtcacaaaagaaagaatgcttggctaatcaagaaaagtatgtggtttgtttcatctaatacggctgtcaaggcccacattggtgttcaaatgcattttttatagttcgtggttactgttaatattctttcctagtagaatgtttggggtttaggtatttttgatgttttatttataaatagatatttatatcttgacgattgtggtgttttatttttttttttttatacatttttcttttcttcctgattcgaaataaaagtatttttagaaaaaggataaagaaacagaaatcaaaagaaataaaaagagtgtggcaaaa

SEQ ID NO: 8 - protein, 717 aa, Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 9 - DNA, 1830 bp, Escherichia coli
  CDS (1)...(1830)
atgtgtggaattgttggcgcgatcgcgcaacgtgatgtagcagaaatccttcttgaaggtttacgtcgtctggaataccgcggatatgactctgccggtctggccgttgttgatgcagaaggtcatatgacccgcctgcgtcgcctcggtaaagtccagatgctggcacaggcagcggaagaacatcctctgcatggcggcactggtattgctcacactcgctgggcgacccacggtgaaccttcagaagtgaatgcgcatccgcatgtttctgaacacattgtggtggtgcataacggcatcatcgaaaaccatgaaccgctgcgtgaagagctaaaagcgcgtggctataccttcgtttctgaaaccgacaccgaagtgattgcccatctggtgaactgggagctgaaacaaggcgggactctgcgtgaggccgttctgcgtgctatcccgcagctgcgtggtgcgtacggtacagtgatcatggactcccgtcacccggataccctgctggcggcacgttctggtagtccgctggtgattggcctggggatgggcgaaaactttatcgcttctgaccagctggcgctgttgccggtgacccgtcgctttatcttccttgaagagggcgatattgcggaaatcactcgccgttcggtaaacatcttcgataaaactggcgcggaagtaaaacgtcaggatatcgaatccaatctgcaatatgacgcgggcgataaaggcatttaccgtcactacatgcagaaagagatctacgaacagccgaacgcgatcaaaaacacccttaccggacgcatcagccacggtcaggttgatttaagcgagctgggaccgaacgccgacgaactgctgtcgaaggttgagcatattcagatcctcgcctgtggtacttcttataactccggtatggtttcccgctactggtttgaatcgctagcaggtattccgtgcgacgtcgaaatcgcctctgaattccgctatcgcaaatctgccgtgcgtcgtaacagcctgatgatcaccttgtcacagtctggcgaaaccgcggataccctggctggcctgcgtctgtcgaaagagctgggttaccttggttcactggcaatctgtaacgttccgggttcttctctggtgcgcgaatccgatctggcgctaatgaccaacgcgggtacagaaatcggcgtggcatccactaaagcattcaccactcagttaactgtgctgttgatgctggtggcgaagctgtctcgcctgaaaggtctggatgcctccattgaacatgacatcgtgcatggtctgcaggcgctgccgagccgtattgagcagatgctgtctcaggacaaacgcattgaagcgctggcagaagatttctctgacaaacatcacgcgctgttcctgggccgtggcgatcagtacccaatcgcgctggaaggcgcattgaagttgaaagagatctcttacattcacgctgaagcctacgctgctggcgaactgaaacacggtccgctggcgctaattgatgccgatatgccggttattgttgttgcaccgaacaacgaattgctggaaaaactgaaatccaacattgaagaagttcgcgcgcgtggcggtcagttgtatgtcttcgccgatcaggatgcgggttttgtaagtagcgataacatgcacatcatcgagatgccgcatgtggaagaggtgattgcaccgatcttctacaccgttccgctgcagctgctggcttaccatgtcgcgctgatcaaaggcaccgacgttgaccagccgcgtaacctggcaaaatcggttacggttgagtaa

SEQ ID NO: 10 - protein, 609 aa, Escherichia coli
MCGIVGAIAQRDVAEILLEGLRRLEYRGYDSAGLAVVDAEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYRHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLGRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 11 - DNA, 1803 bp, Bacillus subtilis
  CDS (1)...(1803)
atgtgtggaatcgtaggttatatcggtcagcttgatgcgaaggaaattttattaaaagggttagagaagcttgagtatcgcggttatgactctgctggtattgctgttgccaacgaacagggaatccatgtgttcaaagaaaaaggacgcattgcagatcttcgtgaagttgtggatgccaatgtagaagcgaaagccggaattgggcatactcgctgggcgacacacggcgaaccaagctatctgaacgctcacccgcatcaaagcgcactgggccgctttacacttgttcacaacggcgtgatcgagaactatgttcagctgaagcaagagtatttgcaagatgtagagctcaaaagtgacaccgatacagaagtagtcgttcaagtaatcgagcaattcgtcaatggaggacttgagacagaagaagcgttccgcaaaacacttacactgttaaaaggctcttatgcaattgctttattcgataacgacaacagagaaacgatttttgtagcgaaaaacaaaagccctctattagtaggtcttggagatacattcaacgtcgtagcatctgatgcgatggcgatgcttcaagtaaccaacgaatacgtagagctgatggataaagaaatggttatcgtcactgatgaccaagttgtcatcaaaaaccttgatggtgacgtgattacacgtgcgtcttatattgctgagcttgatgccagtgatatcgaaaaaggcacgtaccctcactacatgttgaaagaaacggatgagcagcctgttgttatgcgcaaaatcatccaaacgtatcaagatgaaaacggcaagctgtctgtgcctggcgatatcgctgccgctgtagcggaagcggaccgcatctatatcattggctgcggaacaagctaccatgcaggacttgtcggtaaacaatatattgaaatgtgggcaaacgtgccggttgaagtgcatgtagcgagtgaattctcctacaacatgccgcttctgtctaagaaaccgctcttcattttcctttctcaaagcggagaaacagcagacagccgcgcggtactcgttcaagtcaaagcgctcggacacaaagccctgacaatcacaaacgtacctggatcaacgctttctcgtgaagctgactatacattgctgcttcatgcaggccctgagatcgctgttgcgtcaacgaaagcatacactgcacaaatcgcagttctggcggttcttgcttctgtggctgctgacaaaaatggcatcaatatcggatttgacctcgtcaaagaactcggtatcgctgcaaacgcaatggaagctctatgcgaccagaaagacgaaatggaaatgatcgctcgtgaatacctgactgtatccagaaatgctttcttcatcggacgcggccttgactacttcgtatgtgtcgaaggcgcactgaagctgaaagagatttcttacatccaggcagaaggttttgccggcggtgagctaaagcacggaacgattgccttgatcgaacaaggaacaccagtattcgcactggcaactcaagagcatgtaaacctaagcatccgcggaaacgtcaaagaagttgctgctcgcggagcaaacacatgcatcatctcactgaaaggcctagacgatgcggatgacagattcgtattgccggaagtaaacccagcgcttgctccgttggtatctgttgttccattgcagctgatcgcttactatgctgcactgcatcgcggctgtgatgtggataaacctcgtaaccttgcgaagagtgttactgtggagtaa

SEQ ID NO: 12 - protein, 600 aa, Bacillus subtilis
MCGIVGYIGQLDAKEILLKGLEKLEYRGYDSAGIAVANEQGIHVFKEKGRIADLREVVDANVEAKAGIGHTRWATHGEPSYLNAHPHQSALGRFTLVHNGVIENYVQLKQEYLQDVELKSDTDTEVVVQVIEQFVNGGLETEEAFRKTLTLLKGSYAIALFDNDNRETIFVAKNKSPLLVGLGDTFNVVASDAMAMLQVTNEYVELMDKEMVIVTDDQVVIKNLDGDVITRASYIAELDASDIEKGTYPHYMLKETDEQPVVMRKIIQTYQDENGKLSVPGDIAAAVAEADRIYIIGCGTSYHAGLVGKQYIEMWANVPVEVHVASEFSYNMPLLSKKPLFIFLSQSGETADSRAVLVQVKALGHKALTITNVPGSTLSREADYTLLLHAGPEIAVASTKAYTAQIAVLAVLASVAADKNGINIGFDLVKELGIAANAMEALCDQKDEMEMIAREYLTVSRNAFFIGRGLDYFVCVEGALKLKEISYIQAEGFAGGELKHGTIALIEQGTPVFALATQEHVNLSIRGNVKEVAARGANTCIISLKGLDDADDRFVLPEVNPALAPLVSVVPLQLIAYYAALHRGCDVDKPRNLAKSVTVE

SEQ ID NO: 13 - DNA, 2142 bp, Candida albicans
  CDS (1)...(2142)
atgtgtggtatttttggttacgtcaatttcttggtcgacaagagtagaggtgaaatcattgataatttaattgaaggtttgcaacgattagaatatagaggttatgattcagcaggcattgctgttgatgggaaattaactaaagatccttctaatggtgatgaagaatatatggattctattattgttaaaactactggtaaagttaaagttttgaaacaaaaaatcattgatgatcaaatcgatagatcggccatttttgataatcatgttggtattgctcatactagatgggctacacatggtcaaccaaaaactgaaaattgtcatcctcataaatcagatccaaagggggaattcattgttgttcataatggtattattactaattatgctgctttaagaaaatatcttttatcaaaaggacatgtttttgaaagtgaaactgatactgaatgtattgctaaattatttaaacatttttatgatttgaatgttaaagctggtgttttccctgatcttaatgaattgactaaacaagttttgcatgaattagaaggttcttatgggttattagttaaatcttatcattatcctggagaagtttgtggtactagaaaaggttctccattattggttggtgttaaaactgataagaaattaaaagttgattttgttgacgttgaatttgaagctcaacagcaacatcgaccacaacaaccacaaatcaatcataatggtgccacttcagctgctgaattgggctttatcccagtggctccaggtgaacaaaatttaagaacttctcaatcaagagctttcctttctgaagatgatttacctatgccagttgaattctttttatcttctgatcctgcatcagtggttcaacacaccaaaaaagttttatttttagaagatgatgatattgctcatatctatgatggggaattacgtattcatagagcttcgactaaatctgctggggaatctactgttagaccaattcaaactttagaaatggaattgaatgaaattatgaaaggcccctataaacattttatgcaaaaagaaattttcgaacaaccagattctgcttttaatactatgagaggtagaattgattttgaaaattgtgttgttacccttggtggattaaaatcatggttatctacaattagaagatgtagaagaatcattatgattgcttgtggtacttcatatcattcatgtttagccacgagatcaatttttgaagaattgacagaaatccccgtttcggttgaattagcttctgatttcttggatagaagatctccagttttcagagatgatacttgtgtatttgtttctcaatcgggtgaaactgccgactccattttggctttacaatattgtttggaaagaggagctttaactgttggtatcgttaactctgttggttcttcaatgtctagacaaacccattgtggggttcatattaatgctgggccagaaattggtgttgcctcaactaaagcttacacatctcaatatattgccttggtgatgtttgccctttctttatctaatgattctatttccagaaagggaagacatgaagaaattattaaaggtttacaaaaaatccctgaacaaattaaacaagttttgaaattagaaaacaagatcaaagatttatgtaatagttcattgaatgatcaaaaatctttattattattaggtagaggttatcaatttgctactgctttagaaggggctttaaaaattaaagaaatttcttatatgcattctgaaggggtattagctggtgaattaaaacatggtatattagcattagtcgatgaagatttaccaattattgcctttgccactagagattcattatttcctaaagttatgtccgctattgaacaagtcactgctagagatggtagaccaattgttatttgtaatgaaggtgatgctattatttctaatgataaagttcatactactttagaagttccagaaaccgttgattgtttacaagggttattaaatgttattccattacaattgattagttattggttggctgtgaatagaggtattgatgttgatttccctcgtaacttggctaaatcagttactgttgagtaa

SEQ ID NO: 14 - protein, 713 aa, Candida albicans
MCGIFGYVNFLVDKSRGEIIDNLIEGLQRLEYRGYDSAGIAVDGKLTKDPSNGDEEYMDSIIVKTTGKVKVLKQKIIDDQIDRSAIFDNHVGIAHTRWATHGQPKTENCHPHKSDPKGEFIVVHNGIITNYAALRKYLLSKGHVFESETDTECIAKLFKHFYDLNVKAGVFPDLNELTKQVLHELEGSYGLLVKSYHYPGEVCGTRKGSPLLVGVKTDKKLKVDFVDVEFEAQQQHRPQQPQINHNGATSAAELGFIPVAPGEQNLRTSQSRAFLSEDDLPMPVEFFLSSDPASVVQHTKKVLFLEDDDIAHIYDGELRIHRASTKSAGESTVRPIQTLEMELNEIMKGPYKHFMQKEIFEQPDSAFNTMRGRIDFENCVVTLGGLKSWLSTIRRCRRIIMIACGTSYHSCLATRSIFEELTEIPVSVELASDFLDRRSPVFRDDTCVFVSQSGETADSILALQYCLERGALTVGIVNSVGSSMSRQTHCGVHINAGPEIGVASTKAYTSQYIALVMFALSLSNDSISRKGRHEEIIKGLQKIPEQIKQVLKLENKIKDLCNSSLNDQKSLLLLGRGYQFATALEGALKIKEISYMHSEGVLAGELKHGILALVDEDLPIIAFATRDSLFPKVMSAIEQVTARDGRPIVICNEGDAIISNDKVHTTLEVPETVDCLQGLLNVIPLQLISYWLAVNRGIDVDFPRNLAKSVTVE

SEQ ID NO: 15 - protein, 696 aa, Saccharomyces cerevisiae
MASSPQVHPYKKHLMQSQHINFDNRGLQFQNSSLKVGQDFSDNKENRENRDNEDFSTADLPKRSANQPLINEHLRAASVPLLSNDIGNSQEEDFVPVPPPQLHLNNSNNTSLSSLGSTPTNSPSPGALRQTNSSTSLTKEQIKKRTRSVDLSHMYLLNGSSDTQLTATNESVADLSHQMISRYLGGKNNTSLVPRLKTIEMYRQNVKKSKDPEVLFQYAQYMLQTALTIESSNALVQDSDKEGNVSQSDLKLQFLKEAQSYLKKLSIKGYSDAQYLLADGYSSGAFGKIENKEAFVLFQAAAKHGHIESAYRASHCLEEGLGTTRDSRKSVNFLKFAASRNHPSAMYKLGLYSFYGRMGLPTDVNTKLNGVKWLSRAAARANELTAAAPYELAKIYHEGFLDVVIPDEKYAMELYIQAASLGHVPSATLLAQIYETGNDTVGQDTSLSVHYYTQAALKGDSVAMLGLCAWYLLGAEPAFEKDENEAFQWALRAANAGLPKAQFTLGYFYEHGKGCDRNMEYAWKWYEKAAGNEDKRAINKLRSRDGGLASIGKKQHKKNKSISTLNLFSTVDSQTSNVGSNSRVSSKSETFFTGNPKRDREPQGLQINMNSNTNRNGIKTGSDTSIRKSSSSAKGMSREVAEQSMAAKQEVSLSNMGSSNMIRKDFPAVKTESKKPTSLKNKKDKQGKKKKDCVIM

SEQ ID NO: 16 - protein, 671 aa, Saccharomyces cerevisiae
MSSVDVLLTVGKLDASLALLTTQDHHVIEFPTVLLPENVKAGSIIKMQVSQNLEEEKKQRNHFKSIQAKILEKYGTHKPESPVLKIVNVTQTSCVLAWDPLKLGSAKLKSLILYRKGIRSMVIPNPFKVTTTKISGLSVDTPYEFQLKLITTSGTLWSEKVILRTHKMTDMSGITVCLGPLDPLKEISDLQISQCLSHIGARPLQRHVAIDTTHFVCNDLDNEESNEELIRAKHNNIPIVRPEWVRACEVEKRIVGVRGFYLDADQSILKNYTFPPVNEEELSYSKENEPVAEVADENKMPEDTTDVEQVASPNDNESNPSEAKEQGEKSGHETAPVSPVEDPLHASTALENETTIETVNPSVRSLKSEPVGTPNIEENKADSSAEAVVEEPNEAVAESSPNEEATGQKSEDTDTHSNEQADNGFVQTEEVAENNITTESAGENNEPADDAAMEFGRPEAEIETPEVNESIEDANEPAEDSNEPVEDSNKPVKDSNKPVEDSNKPVEDSNKPVEDSNKPVEDANEPVEDTSEPVEDAGEPVQETNEFTTDIASPRHQEEDIELEAEPKDATESVAVEPSNEDVKPEEKGSEAEDDINNVSKEAASGESTTHQKTEASASLESSAVTEEQETTEAEVNTDDVLSTKEAKKNTGNSNSNKKKNKKNKKKGKKK

SEQ ID NO: 17 - protein, 746 aa, Saccharomyces cerevisiae
MNLFWPSETKKQNEIPGGDYTPGNSPSVQKGYQFLNRDIFKSCPRIMERQFGECLHNRTHLIKDLISSGNVGLGPIEIVHMSYLNKHEKEEFGEYFYVTGIEVSGPAMPVEFLEVLKSSKRISKNISNNIILTYCCFNFFSNLDIRIRYDADDTFQTTAIDCNKETTDLTMTEKMWEETFASSVIRAIITNTNPELKPPGLVECPFYVGKDTISSCKKIIELLCRFLPRSLNCGWDSTKSMQATIVNNYLMYSLKSFIAITPSLVDFTIDYLKGLTKKDPIHDIYYKTAMITILDHIETKELDMITILNETLDPLLSLLNDLPPRDADSARLMNCMSDLLNIQTNFLLNRGDYELALGVSNTSTELALDSFESWYNLARCHIKKEEYEKALFAINSMPRLRKNDGHLETMYSRFLTSNYYKKPLNGTREHYDLTAMEFTNLSGTLRNWKEDELKRQIFGRIAMINEKKIGYTKEIWDDIAIKLGPICGPQSVNLINYVSPQEVKNIKNINLIARNTIGKQLGWFSGKIYGLLMEIVNKIGWNGLLNIRTEAFMMETEFYQASNNIIDENGHIPMESRKKRFCEGWLDDLFLDLYQDLKLSKISLSNKDEKHSGLEWELLGLIMLRTWHWEDAVACLRTSIVARFDPVSCQQLLKIYLQPPKNIQEVTLLDTDTIISLLIKKISYDCRYYNYCQIFNLQLLEKLCNELGTHILRNKILLQPSIGDEIMVMIDAMLAWIADLDHTVQP

SEQ ID NO: 18 - protein, 316 aa, Saccharomyces cerevisiae
MAFSDFAAICSKTPLPLCSVIKSKTHLILSNSTIIHDFDPLNLNVGVLPRCYARSIDLANTVIFDVGNAFINIGALGVILIILYNIRQKYTAIGRSEYLYFFQLTLLLIIFTLVVDCGVSPPGSGSYPYFVAIQIGLAGACCWALLIIGFLGFNLWEDGTTKSMLLVRGTSMLGFIANFLASILTFKAWITDHKVATMNASGMIVVVYIINAIFLFVFVICQLLVSLLVVRNLWVTGAIFLGLFFFVAGQVLVYAFSTQICEGFKHYLDGLFFGSICNVFTLMMVYKTWDMTTDDDLEFGVSVSKDGDVVYDNGFM

SEQ ID NO: 19 - DNA, 567 bp, Aspergillus niger
  CDS (1)...(567)
  misc_feature 441, 474, 527: n = A,T,C or G
gatttcctcttcgctcggacgatgatcggtgtgttcaagaacattgagtacatgtgctccaggacatcaagcaagacgtggggcaaagatgcctggaagaagattgtggtttgtgtcatcagtgacggtcgtgcgaagatcaatccgcgaacgcgcgctgtccttgctggtttgggatgctatcaggatggaattgccaagcagcaagttaacggcaaggatgtcacagcacacatctacgaatataccacgcaggtgggtctagaattgaagggaggccaagttagcctcaagcctcgaactggatgccccgttcagatgatcttttgtctgaaggaaaagaaccagaagaagatcaactcgcaccgctggttcttccaggcatttggtcgcgtgctggatcccaatatctgcgttctgttggacgcgggtacccggccnggaaaggactcgatctaccatctttggaaggcnttcgatgttgacccgatgtgtggaggtgcttgtggtgagatcaaggtcatgtngtcgcatggaaagaagctgctcaacccgttggttgccggg

SEQ ID NO: 20 - protein, 189 aa, Aspergillus niger
  VARIANT 176: Xaa = Any Amino Acid
DFLFARTMIGVFKNIEYMCSRTSSKTWGKDAWKKIVVCVISDGRAKINPRTRAVLAGLGCYQDGIAKQQVNGKDVTAHIYEYTTQVGLELKGGQVSLKPRTGCPVQMIFCLKEKNQKKINSHRWFFQAFGRVLDPNICVLLDAGTRPGKDSIYHLWKAFDVDPMCGGACGEIKVMXSHGKKLLNPLVAG

SEQ ID NO: 21 - DNA, 567 bp, Aspergillus niger
  CDS (1)...(567)
gagatcggcttcactcggactctgcatggcgtgatgcagaatatcacccacctttgctcgcgatccaagtcccgtacctggggtaaggatggttggaagaagattgtggtctgcatcatcgccgacggtcgtaagaaggtgcacccgcggaccctcaatgctttggcggctttgggtgtctaccaggaaggtatcgccaagaacgttgtcaaccagaagcaggtcaacgcgcacgtctacgaatacacgacgcaggtatcgctcgatcccgacttgaagttcaagggcgctgagaagggcatcatgccgtgccaggtgctcttctgtttgaaagaacacaacaagaagaaattgaactcacaccgttggttcttcaacgcttttggccgagcactacagccgaacatttgcatcctgctcgatgtgggtaccaaacctgcgcctaccgcactctaccacctctggaaagcgttcgaccagaactcgaacgtcgcgggagcggctggtgaaatcaaggcgggcaaaggcaagggtatgctcggtcttctgaaccctctggttgccagt

SEQ ID NO: 22 - protein, 189 aa, Aspergillus niger
EIGFTRTLHGVMQNITHLCSRSKSRTWGKDGWKKIVVCIIADGRKKVHPRTLNALAALGVYQEGIAKNVVNQKQVNAHVYEYTTQVSLDPDLKFKGAEKGIMPCQVLFCLKEHNKKKLNSHRWFFNAFGRALQPNICILLDVGTKPAPTALYHLWKAFDQNSNVAGAAGEIKAGKGKGMLGLLNPLVAS

SEQ ID NO: 23 - DNA, 2670 bp, Aspergillus fumigatus
  CDS (1)...(2670)
atgggcactccaaggccctattcggcgcattcaccgcaggaaagccgaagcagcttctactcgcagccctcccaatctcctacacagcctacctacggtcgcgatgacgcggaagatcaacaacaatccctccttcgacgcagtttagccagtcccaatggctggtcatacgatgatcccaacgtatctaccgactctctgaggcgatacacattgcatgatccggggataaccgcatttgctcccccgtacccggagtccgaggccgccgatgtacgcagcgcacggatgtcgggatacagtggaatcgagatggatgcctggcaaagacgacaaggggtgaaaccaagtgcgctacggcgatatggaaccaggaagatcaacctcgtccagggttcggtactcagcgttgactacccggtgccgagtgcgattcagaacgcgatccaggcggagtatcgggatgcggaggaagcgtttcatgaagaattcacgcatatgcggtatactgccgccacctgcgacccagatgaattcactctgcgcaacggctacaacctacgtcccgctatgtataaccgtcatacggaacgtctcatcgcgatcacttattataatgaggataaagttctgaccgcccgaactctgcatggagtgatgcaaaacgtccgggacattgtgaacctgaagaagtcagaattttggaacaagggaggaccggcatggcagaagattgtggtgtgtctcgtgtttgatggaattgagccttgcgataagaatactctggatgtcctcgccacgataggtgtgtatcaggatggggtgatgaaaaaagatgtggacgggcgcgagacagtcgcacatattttcgagtatacgacacaattatccgtcaccccgacacagcagctggtcagaccgcagcctaatgatcctagcaatctccccccggtccagatgctattctgcctcaagcagaagaacagcaagaaaatcaattcccaccggtggctgttcaacgcctttagtcgaatcctgaatccggaaatatgcatcctgcttgatgctggcacgaagcccgggagcaaatccttgcttgccctatgggaagcattctataatgacaaaacactgggcggagcatgcggcgaaatccatgccatgctgggcagggggtggcgcaatgtgctgaaccctctagtcgcagcgcagaattttgagtacaaaatttccaatattcttgataaaccgctggaaagcgcctttggctacgtgagtgtgctaccgggtgctttctcggcatatcgctaccgggcgatcatgggccgaccgttggagcaatactttcatggggatcatactttgtccaagcggctgggaaagaagggcattgaggggatgaatatctttaaaaagaacatgtttcttgcagaggatcgcatcctatgctttgaactggtggccaaagctgggtataaatggcatctcacgtatgtgaaagcatccaagggagagacagatgttcccgaggcggcgcccgaatacatcagtcagcggcgacgatggctcaatggctcctttgctgcgagtttgtattccatcatgcacttcggacgcatctataagagtggtcatagcttcgttcgaatgttcttcttgcatattcagatgatttacaactgctgccagctcatcatgacctggttctcgttggcatcctactggctgaccagctcggtcatcatggacctcgtagggacgcccagctcgcataacaagtacaaggcatggccattcggcaacgatgcctcccccattgtcaacttttttgtcaaatatggttatctcttggtgctgatgctccaatttgtgctggctctgggaaaccgccccaaagcctacaccatgtcgttcctctggttttctctggtgcagttctacgtgctgatcctgtccttctacctggtcgctaatgccttcatgggtggcatgatcgactttgatttcgaccaaggcgtgggcaacttcctctcctccttcttcagctcgactggtggagggattgtcctgatcgccttggtgtccacttacggcatttacattgtcgcgagcattctctacatggacccttggcacatcctgaccagttcctgggcatacttcctgggcatgaccacgtcgatcaatattctcatggtgtatgcgttctgcaactggcacgatgtgtcgtggggtacgaaggggtccgataaggcggacgctctgccctcagcgcagacgaagaaggccgacgcgagcaagagcaacttcatcgaggagatcgataaaccgcaggcagacatcgacagccagttcgaggcaacggtcaaacggcggctggccccgtatcaggagcccaaagaagactcgactatcagcctggatgattcctaccgcaactttcggaccagcctggtgttgctctggatcttgagtaatttgctggtctctctgctcattacgagcgacggcatcaggaagatgtgtctgacgaacacatccacaacgcggacgcagtattatttccaagtgattttgtgggcgacagctggactgtctatcttccgattcattgggtcgatctacttcctgggcaagtcgggaatcttatgttgcgtaacgcgtcgatga

SEQ ID NO: 24 - protein, 889 aa, Aspergillus fumigatus
MGTPRPYSAHSPQESRSSFYSQPSQSPTQPTYGRDDAEDQQQSLLRRSLASPNGWSYDDPNVSTDSLRRYTLHDPGITAFAPPYPESEAADVRSARMSGYSGIEMDAWQRRQGVKPSALRRYGTRKINLVQGSVLSVDYPVPSAIQNAIQAEYRDAEEAFHEEFTHMRYTAATCDPDEFTLRNGYNLRPAMYNRHTERLIAITYYNEDKVLTARTLHGVMQNVRDIVNLKKSEFWNKGGPAWQKIVVCLVFDGIEPCDKNTLDVLATIGVYQDGVMKKDVDGRETVAHIFEYTTQLSVTPTQQLVRPQPNDPSNLPPVQMLFCLKQKNSKKINSHRWLFNAFSRILNPEICILLDAGTKPGSKSLLALWEAFYNDKTLGGACGEIHAMLGRGWRNVLNPLVAAQNFEYKISNILDKPLESAFGYVSVLPGAFSAYRYRAIMGRPLEQYFHGDHTLSKRLGKKGIEGMNIFKKNMFLAEDRILCFELVAKAGYKWHLTYVKASKGETDVPEAAPEYISQRRRWLNGSFAASLYSIMHFGRIYKSGHSFVRMFFLHIQMIYNCCQLIMTWFSLASYWLTSSVIMDLVGTPSSHNKYKAWPFGNDASPIVNFFVKYGYLLVLMLQFVLALGNRPKAYTMSFLWFSLVQFYVLILSFYLVANAFMGGMIDFDFDQGVGNFLSSFFSSTGGGIVLIALVSTYGIYIVASILYMDPWHILTSSWAYFLGMTTSINILMVYAFCNWHDVSWGTKGSDKADALPSAQTKKADASKSNFIEEIDKPQADIDSQFEATVKRRLAPYQEPKEDSTISLDDSYRNFRTSLVLLWILSNLLVSLLITSDGIRKMCLTNTSTTRTQYYFQVILWATAGLSIFRFIGSIYFLGKSGILCCVTRR

SEQ ID NO: 25 - DNA, 2238 bp, Aspergillus fumigatus
  CDS (1)...(2238)
atgattgtcctcttcacgttactgaggtgggctccaatatccccagtcttctctatgaggaccatgcatgctaatttggcacacaggggaattttcttgcctgtcatgatcgtgaccctcccgctcccggttcacttgagacggcgattcccggctcagatggtacttatgcttcaatggttcgcgttcgggatgttttccgtgctgcttataatcccttggcttttgtgcgtctacagactggtgacacattcaccgggcagaaccaagcgtatcaagcaagttttggatgaccgaaccgctcccaaaacagttgttgttatgccagtctataaggaagccccggaaacactaataagggcaatcgattctgtcgttgactgtgattatccagccaactgtatccatgtgttcctctcttacgatggctgcctcattgacgaatcctatcttcggctgattgaacaccttggaattccgattacgctggagagctatccacagagcatagacgtgacgtacaaagacgccagaattacggtctctcgtttcaaacatggagggaaacgacattgccagaagcaaacgttcagactgattgacatggtatatgcggattacctggagcgccacgacaaccttttcgtgttattcattgactccgactgcatccttgaccgtgtatgtctgcaaaacttcatgtacgatatggagttgaagccagggagcaaacacgacatgttggcaatgacgggggtcattacgtcgactacggaccgaggctcgctcctcacacttctgcaggacatggagtatgtccatgggcaactgttcgagcgctctgtggaatctagctgcggcgctgtgacttgcctccccggggctctgacgatgctccggttctctgcgtttcgtaaaatggccaagtactacttcgcggacaaagccgagcaatgcgaggacttttttgactatggcaagtgtcatcttggagaagatcgctggctcacgcacctcttcatggtaggcgctcggaaacgttatcaaatccagatgtgcgcaggcgccttttgtaagaccgaggcagtgcagacattcagcagtcttttaaagcagcgtcggcgctggtttctgggtttcataaccaacgaagtgtgtatgctgactgatgtgcggctttggaagcgctaccccttgctctgcctggtccgttttatgcagaacacgatccgaacaactgcattgctgttttttatcatcgcgctgtcacttataacaacctcgagcagcatcaatgacctgcccgtgggttttattgccatatcgctgggactcaactacgtcctcatgttctacttgggtgcgaagctcaagcgctacaaagcttggctttttccgctgatgtttatcctgaatcccttcttcaactggctgtatatggtgtatggaatcctgactgcgggccagcgtacatggggaggaccgagagccgatgccgccaccgccgatgagcacacttcgcccgaggaagctgttgaactggctaaggctcaaggcgatgagctcaatgtcgatctgactactttccgttccagaggggatgagaagagcgttccaatccatccctcggagaagatcgatgggcgcttctctgcaccagagctcccagacggttatgactcgaacttgaacgactccaacgcagcccttaccgagctgatgacgcctcttccgagtgtgcctcggataggtatccatacatacccgtcctccgattcgatcctaacctcggactcgctgagctcgatccaccttccccttaaggttgaagagctgactggtgataatgacaatatgaagccctatcccgatcggcaaccaagggacacgtcgagtttgcaccagatgcagaggacttgttctaacggaatcgtggccagtgattcatgctcttcacaggacgatgcttcggagatggtaaacaagcctgagatactgtcaccatcagctcatatactgccgcatccatcacaagccacggagtcgtcatccggggaggatatatacccacttcatttgccatcgccacaccaacacgaggcacattttgctcctctcaatgcttcaacccgaggttcaatggaaggaaacaccccagaggtacagcgaccacgccgtaagcttccagggattccacggcctatcagagcccagaaagatcctgaaagtatggtctag

SEQ ID NO: 26 - protein, 745 aa, Aspergillus fumigatus
MIVLFTLLRWAPISPVFSMRTMHANLAHRGIFLPVMIVTLPLPVHLRRRFPAQMVLMLQWFAFGMFSVLLIIPWLLCVYRLVTHSPGRTKRIKQVLDDRTAPKTVVVMPVYKEAPETLIRAIDSVVDCDYPANCIHVFLSYDGCLIDESYLRLIEHLGIPITLESYPQSIDVTYKDARITVSRFKHGGKRHCQKQTFRLIDMVYADYLERHDNLFVLFIDSDCILDRVCLQNFMYDMELKPGSKHDMLAMTGVITSTTDRGSLLTLLQDMEYVHGQLFERSVESSCGAVTCLPGALTMLRFSAFRKMAKYYFADKAEQCEDFFDYGKCHLGEDRWLTHLFMVGARKRYQIQMCAGAFCKTEAVQTFSSLLKQRRRWFLGFITNEVCMLTDVRLWKRYPLLCLVRFMQNTIRTTALLFFIIALSLITTSSSINDLPVGFIAISLGLNYVLMFYLGAKLKRYKAWLFPLMFILNPFFNWLYMVYGILTAGQRTWGGPRADAATADEHTSPEEAVELAKAQGDELNVDLTTFRSRGDEKSVPIHPSEKIDGRFSAPELPDGYDSNLNDSNAALTELMTPLPSVPRIGIHTYPSSDSILTSDSLSSIHLPLKVEELTGDNDNMKPYPDRQPRDTSSLHQMQRTCSNGIVASDSCSSQDDASEMVNKPEILSPSAHILPHPSQATESSSGEDIYPLHLPSPHQHEAHFAPLNASTRGSMEGNTPEVQRPRRKLPGIPRPIRAQKDPESMV

SEQ ID NO: 27 - DNA, 2736 bp, Aspergillus fumigatus
  CDS (1)...(2736)
atggcctaccaaggctctggttctcattcgccgcctcactacgacgataacggtcaccgacttcaggatctgcctcatggttcgtacgaagaagaggcgtcgagaggattgctatcccaccagcagggtcccttcacagggccgtttgatgaccctcagcagcatggttcatctactaccagacccgtttctggatacagtttgagcgagacctatgcccccgaagccgcatatcatgatccctatactcaaccgagccctggctcggtctactcggctcaatctgcggagaatccggcggcggcttttggtgtccctggacgtgtcgcgtccccctatgctcgaagtgacacttcatccacagaggcatggcgccagagacaagctcctggaggcggccccggtgggttgcggcgttatgctacaagaaaggtcaagttggtgcagggttccgtcctgagtgtcgattatcctgttccgagcgcgatccagaatgctattcaggccaagtacagaaatgatctcgagggtggaagtgaggaatttacgcacatgcgatacaccgcggccacctgtgatcccaatgaatttacactgcacaatggttacaatcttcgacctgcgatgtacaacagacataccgaacttctcattgcgatcacctattataacgaagacaagacactcacttcacgtacactgcacggtgtcatgcagaacattcgcgacattgtgaatctgaaaaagtccgagttctggaacaagggtggtcctgcctggcagaagattgtggtctgcctggtttttgatggtatcgacccttgcgacaaggacactttggatgttcttgcgacaatcggtgtctaccaagacggcgtcatgaagcgtgacgtcgatggaaaagagactgtggctcacatttttgaatatacgactcaactgtctgtgactccgaatcagcagctgatccgccccaccgacgacggtcccagtacacttcttccgtccaagatgatgttctgtttgaagcagaagaacagcaagaagatcaactcccacagatggctcttcaatgcatttggtcgcattctcaaccccgaggtctgtattcttcttgacgccggtacaaagcctggcccgaagtcacttctgtcgctttgggaagccttctataacgataaagatttgggtggtgcttgcggtgagattcacgccatgttgggtaagggttggaagaacctgatcaacccgctcgttgccgctcagaacttcgaatataagattagtaacatcctggacaagcccttggagagttcatttggttatgtcagcgtcttgcctggtgccttctcagcttaccgattccgcgcaattatgggccgccctctcgagcagtatttccatggtgaccacactctttcaaagcaactgggcaagaagggtatcgagggaatgaacattttcaagaagaacatgttcttggccgaagatcgtattctttgttttgagttggttgctaaagctggttcaaaatggcacctgacctatgtcaaggcctccaaggctgaaaccgacgtgccagaaggcgctcccgaattcatttcgcagcgtcgtcgttggctgaacggttcgtttgctgccggtatttattcgctcatgcactttggtcggatgtacaagagtggacacaacattgtccgcatgttttttctgcacatccaaatgttgtacaatatcttctctacggttctgacatggttttccttggcttcttactggcttactaccaccgtcatcatggacttggtcggcacgccgagcgacaataacggtaacaaggctttcccgttcggcaagactgcaactcccattatcaataccatagtgaaatatgtctacttgggattcctactattgcaattcatcctcgcgctgggtaaccgtccaaaaggatcgaagttctcatacctcgcgtctttcgtggttttcggcatcattcaggtctacgtggttattgatgcactgtacctggtggtgcgtgccttcagtggaagtgctcctatggatttcactacggaccaaggcgttggcgagtttctgaaatcgttcttttcatccagcggcgccggtatcattatcattgctctcgctgctacattcggtctctactttgtggcatcttttatgtatcttgacccctggcacatgttcacgtccttccctgcctacatgtgtgtccaatcgtcatacatcaatattctgaacgtctacgctttcagcaattggcacgacgtctcgtggggtaccaagggttcagacaaggcagacgctctaccttccgccaagaccaccaaggatgagggcaaagaggttgtcatcgaggaaatcgacaagcctcaggctgatatcgacagtcagttcgaggcaacggtcaagcgtgccctaacaccctatgtgccaccagtcgagaaggaggaaaagactctggaagactcgtacaaaagcttccgaacgagactggtcacgttttggatctttagcaacgccttcttggccgtttgcatcaccagtgacggtgtggataaattcggcttcacgaattctgctaccgaccggacgcagcgtttcttccaggctttgctgtggtccaacgctgtcgttgccctgttccgtttcatcggagcctgctggttcctgggcaagacaggtttgatgtgctgctttgcccggcgttag

SEQ ID NO: 28 - protein, 911 aa, Aspergillus fumigatus
MAYQGSGSHSPPHYDDNGHRLQDLPHGSYEEEASRGLLSHQQGPFTGPFDDPQQHGSSTTRPVSGYSLSETYAPEAAYHDPYTQPSPGSVYSAQSAENPAAAFGVPGRVASPYARSDTSSTEAWRQRQAPGGGPGGLRRYATRKVKLVQGSVLSVDYPVPSAIQNAIQAKYRNDLEGGSEEFTHMRYTAATCDPNEFTLHNGYNLRPAMYNRHTELLIAITYYNEDKTLTSRTLHGVMQNIRDIVNLKKSEFWNKGGPAWQKIVVCLVFDGIDPCDKDTLDVLATIGVYQDGVMKRDVDGKETVAHIFEYTTQLSVTPNQQLIRPTDDGPSTLLPSKMMFCLKQKNSKKINSHRWLFNAFGRILNPEVCILLDAGTKPGPKSLLSLWEAFYNDKDLGGACGEIHAMLGKGWKNLINPLVAAQNFEYKISNILDKPLESSFGYVSVLPGAFSAYRFRAIMGRPLEQYFHGDHTLSKQLGKKGIEGMNIFKKNMFLAEDRILCFELVAKAGSKWHLTYVKASKAETDVPEGAPEFISQRRRWLNGSFAAGIYSLMHFGRMYKSGHNIVRMFFLHIQMLYNIFSTVLTWFSLASYWLTTTVIMDLVGTPSDNNGNKAFPFGKTATPIINTIVKYVYLGFLLLQFILALGNRPKGSKFSYLASFVVFGIIQVYVVIDALYLVVRAFSGSAPMDFTTDQGVGEFLKSFFSSSGAGIIIIALAATFGLYFVASFMYLDPWHMFTSFPAYMCVQSSYINILNVYAFSNWHDVSWGTKGSDKADALPSAKTTKDEGKEVVIEEIDKPQADIDSQFEATVKRALTPYVPPVEKEEKTLEDSYKSFRTRLVTFWIFSNAFLAVCITSDGVDKFGFTNSATDRTQRFFQALLWSNAVVALFRFIGACWFLGKTGLMCCFARR

SEQ ID NO: 29 - DNA, 2751 bp, Aspergillus orzae
  CDS (1)...(2751)
atggcctaccaaccgcctggtaaagataatggtgctcagtcaccaaactacaacgatagcggtcatcgactggaagacctgccccatggcgccacttatgaagaagaagcttcaacaggactgctttcccaccaacagggcggtcctttcggtggtcctttcgacgaccctcatcagcgtggcacctcgcctgttcgacctacgtcgggatacagcttgactgaaacatacgctccggacgcgggttttcatgacccttacagcacgacgggctcggtttactccggcaactcggcagaaaaccccgcggctgcctttggcgtcccgggtcgtgtagcttctccctacgctcgcagtgaaacatcctccacagaagcatggcgccagcgccaggctccaggaggtggtggcggtggtggcctccgtcgttacgctaccagaaaggtcaagctagttcagggttccgtcctcagtgtcgattaccccgtccccagtgctatccagaatgccatccaagccaagtaccgtaatgacctggagggtggcagcgaggagtttacccacatgcgatacaccgctgcgacatgtgatcccaatgacttcaccctgcacaatggttacaatctgcgtcccgccatgtataacagacacaccgagttgttgattgcgattacctattataacgaagataagacccttaccgctcgtaccttgcacggtgtgatgcagaacattcgcgacattgtgaacctcaagaagtccgagttctggaacaaaggtggacccgcctggcagaaaattgttgtcgctctggtctttgacggtatcgatccttgtgataaggacactttggatgtcctcgccaccatcggtatctatcaggacggtgtcatgaagcgtgacgtcgacggcaaggagaccgtggctcatattttcgaatacacgacccaattgtccgtgacaccgaaccagcaactcatccggcccactgatgatggcccgaccaccttgcccccggtgcagatgatgttctgcttaaagcagaagaacagcaagaagatcaactcacacagatggctgttcaatgccttcggtcgtattttgaatccagaggtttgcatccttctggatgccggtactaagcctggccagaagtccctcctggcgttgtgggagggcttctataacgacaaggatctgggaggtgcttgtggtgaaattcacgcaatgttgggtaaaggctggaagaatctgatcaaccccctcgtcgcggcccagaacttcgagtacaagatcagtaatatcctcgataaacccttggagagttctttcggttatgtcagtgtgttgcctggtgctttctccgcctatcgtttccgtgccatcatgggtcgacccctcgaacaatatttccatggtgatcacactctctcaaaacagctgggtaagaagggtattgagggcatgaacatcttcaagaagaacatgttcttggccgaagatcgtatcctttgtttcgaactggtcgccaaggctggctccaaatggcacttgtcctacatcaaagcctcgaagggtgaaactgacgtgccggaaggtgttgctgaattcatttcccagcgtcgtcgttggttgaacggttcttttgcggccggtctctattcgctcatgcatttcggtcggatgtacaagagtggacataacatcatccgtatgttcttcttgcacattcagatgttgtacaacgttttcaacactatccttacatggttctccctggcatcttactggttgaccaccaccgtcatcatggacttggtcggaacgcccagtgagagcaacggtaacaaaggattccccttcggtaaatcggcgacccctattatcaacacaattgtgaagtatgtctacctcggattgttgctcttgcagttcattctcgctctcggtaaccgccccaagggatcccgcttctcgtacctgacatctttcgtcgtattcggtatcattcaaatctacgttgtcgtcgacgctctgtacttggtggttcgtgcattcacaaacagtgatgcgatagatttcgtcaccgatcaaggtgttggcgagttcctcaagtcgttcttctcgtcttccggcgccggtatcattatcatcgccctggctgctactttcggtctctacttcgtcgcttcgttcatgtacctggacccttggcatatgttcacctcgttccccgcctacatgttcgttcagtcatcttacatcaacgttctcaacgtgtacgcgttcagcaactggcacgatgtctcgtggggtaccaagggttctgataaggccgatgcgctcccttctgcaacgactacgaaggaggatggcggcaaggaagctgtcattgaggaaatcgacaagccccaggctgatattgacagccaatttgaagccactgtcaagcgcgctctcaccccctacgtcccccctgtggagaaggatgagaagtccttggatgattcctacaagagtttccgtacccgtcttgtgacgttgtggatcttcagtaatgccttcttggctgtatgcattaccagtgacggtatggacaagtttggattcacgaacaccgctaccgaccgtacgtcgcgtttcttccaggctctcctgtggtccaacgctgctgtcgcacttgtccgtttcattggtgcctgttggttcctgggtaagacgggtctcatgtgctgcttcgctcgtcggtag

SEQ ID NO: 30 - protein, 916 aa, Aspergillus orzae
MAYQPPGKDNGAQSPNYNDSGHRLEDLPHGATYEEEASTGLLSHQQGGPFGGPFDDPHQRGTSPVRPTSGYSLTETYAPDAGFHDPYSTTGSVYSGNSAENPAAAFGVPGRVASPYARSETSSTEAWRQRQAPGGGGGGGLRRYATRKVKLVQGSVLSVDYPVPSAIQNAIQAKYRNDLEGGSEEFTHMRYTAATCDPNDFTLHNGYNLRPAMYNRHTELLIAITYYNEDKTLTARTLHGVMQNIRDIVNLKKSEFWNKGGPAWQKIVVALVFDGIDPCDKDTLDVLATIGIYQDGVMKRDVDGKETVAHIFEYTTQLSVTPNQQLIRPTDDGPTTLPPVQMMFCLKQKNSKKINSHRWLFNAFGRILNPEVCILLDAGTKPGQKSLLALWEGFYNDKDLGGACGEIHAMLGKGWKNLINPLVAAQNFEYKISNILDKPLESSFGYVSVLPGAFSAYRFRAIMGRPLEQYFHGDHTLSKQLGKKGIEGMNIFKKNMFLAEDRILCFELVAKAGSKWHLSYIKASKGETDVPEGVAEFISQRRRWLNGSFAAGLYSLMHFGRMYKSGHNIIRMFFLHIQMLYNVFNTILTWFSLASYWLTTTVIMDLVGTPSESNGNKGFPFGKSATPIINTIVKYVYLGLLLLQFILALGNRPKGSRFSYLTSFVVFGIIQIYVVVDALYLVVRAFTNSDAIDFVTDQGVGEFLKSFFSSSGAGIIIIALAATFGLYFVASFMYLDPWHMFTSFPAYMFVQSSYINVLNVYAFSNWHDVSWGTKGSDKADALPSATTTKEDGGKEAVIEEIDKPQADIDSQFEATVKRALTPYVPPVEKDEKSLDDSYKSFRTRLVTLWIFSNAFLAVCITSDGMDKFGFTNTATDRTSRFFQALLWSNAAVALVRFIGACWFLGKTGLMCCFARR

SEQ ID NO: 31 - DNA, 6321 bp, Aspergillus orzae
ttcctattctatccgccttccaattgtccgcctctgttgttgttgttgtttggatctctcttgcttgctttttccttcgttacgggagtagtgctttcgagtgtgtttgatctttctatcttatttcatttctttttcttttgataaccgaatcgtttagcccttcgctttcgcctcatcggttgtaattataatctctgggctttacaaacgtcgctttgcccaacactcgctgctcatacacattcccgtcactcgcttcataacctgaagaatactacgacatcgtttctaacgatcacagcttctgaatttactattaatcacttccccgctcagattgtattcgcatatccttcattcggctcacgaccaaagtgacgctaggaccgtgtgcgcatacctggatccttcacctaggggtgaagtcagaactgaaaacaggaaggctgtagtttcatgctctgaacgtataagattcggttcgtcgttcgttccatcgcttggttgacgttcattacgagagcacatcggtgatagaccgccgattaacctcggggttacacaaccatgtcaaatcgttattcagtttactccagccattcggctgctttctccaccggtggccgagcgccccaggcaggtgggcaggtgtcaacgacgacgttgctgaatgcgttgcatgcgcactatacgacgggacaaccctaccaactcgacgcgggaaccagtctagtcgttaatacattgcttactgcaacacaatcgtccccagaaggccataccgggcctacaatagatcacgaactagcagtccgagcctgggaacatgcaaggagaagagcagaggacggttgtattgttctatggttagcgttcatcccgagatccagcagtatcggaagatcaactaacattcaatcataaccagttctgcccaccactcagcaccatccattttagagccattcttagcagcactacctttgtccactcccagtatcgccttcacagcgttggccgctctccgtccctttttgacggctgtaaccaccttcaacccttcatactcgctctattccgcgctttcagcatgctataacttgaccctcaagggcgacatcaccgggctatctctagcactttcgacatcagggatcaatgtccgcaaaggattcctcgacattccttctgagccggggtatcgtgccttcgatgtgttctattatctcttaacgtctgcatcgacaccagctgagcgggagtttttggacctgagagacgcctcttcctacgctttgctccgcaagtctggtacatatacacccccatcatatcttcctactgccgacgacgctgccgctgccgaagactttagatctgctctcaaggccatcggcatcaaaggtgcatctcaacgaggtcttttgtcagtacttgctgggttactcaaacttggtaatgcggctggcttcctagttgaccaggaagacctggaggaggcgtgtgaagatgtaggtggtttactgggcatagatcccgaagttcttctacacaagtgctccaccgacgacagagaggtgttgatttctggaatctacgaggctctggttgattgggtgatcggaaaagccaatgaagccatagcgagccagcttcaggcgagcttggatgatagcagtcgtggttctggacaggcagctcagtggaccgacgatgacacggtgagcatcaccgtggttgacctccctaggcctgcgctgggaaaagctgtggcgatgagagggatattcgacgataccctcggtatcaacgctgaaatgaaagatgacggggtcgtcgttcctccagctggcccagccgtcctgaatgatatgactgcagcgattgcccaggttgaagtcgatctgggaataaccactggccctacgtggcgcgaacgagagtatgaacttgacaaaaaacacgaagtcctggagaaagttgggcttgaagtcgaaatggactcgtttcttcggcagattttgtttaccgctgaatccgaaggaatcacactgggtaagaaaggccgatttgatttggctaccacactaggcagcagccgtgtttggcatcatatttccattcatcccaccgatgacttacctgagaatctgagccctggtgtgccgactgcggcttggtctgcaggcgccgtttcccgtcaactacgggaatggcgactcgcggagtgggcaaatcgtcgccttaaacagatcgacttcacagccgactttgacatagaagaattcatcggtagatacttccgacttggttgcggggaaggaaaagacggtgttgagaactggttagttgaaagggggtggatcaacggcgatgccgtggttggccatcagcgaatctgggtgagagaaaatgcgtggtgggaagcagagacaatgctcgatttgaagcctgaagagccaccagcagcaagccctttcatgtatggaggtggcatgcttgatcctggcgttccgcattacgcagtgcctcccatcgcagaatctacgagccttctagggagccgggataatcttctcaacagacaaagtacactggttcccagtgttgctggtggtgccaagtcgatcgcgcccagcgctccgcataccttgcacacgggcggtgattacggcttaggcacaaagggagatgataaaaagtatgacagccacccctactacgatgacgagggccgctacttaggcgagcttgaccccgagtatgccgaccccaagcatattgagaaaaaggagattactctcggtcgacgtatatggactggctttgtctgggccttgactttctggattccatcgttcgtccttcgcttcgtgggccggatgaagcgtcctgatgtgcgaatggcgtggagagaaaagctcgtcctcgtgttcctcattcttctcttcaacgctattgtctgcttctatatcatcgccttcggtaacttgctttgtccaaacaaggacaaagtctggaacgagaaagaggtcagttaccatcaagggaataatgacttttacgtcagtattcacggcaaagtctacgacatcagtaagttctggaagattcaacacagtgatacaagcattgagacgaccacatcgaacatggagcctttcatgggtgaaaaccttgacgcttattttcctcctccgcttactcgactctgtggtgactttgtgaccgatgagtctatcacactgagaaacaacgacacaaacgcggtgttgtattcaaatgccaaacacagttgtggccctctccagcaaacggacccgaacacagcactgcacaagatcacctggtacgaagatgtctttctgccaaagattgacgagtactataaggggtcccttgtatggaagcggagtgaggtgtcaaaacaagcagacagcagctcccggtactgggtcatcaaggatgaaagcatttatgatttgacagactacttctataccctcaagcagatgaacaacatagacagttacaatttcctaccaagcagcatcacggaacttttcaaaaactatccaggcactgacgtgacagacaaatggccaaacagtgaaaatgccaccaaagcgcaaacatgtctcgattacgtgttctataaaggcaaagtcgatttccgtgatagtgcgagatgccaggtcaataattacattctgttagcattcacgtgtctcatttgtgcagtcattctcgtcaagttcctcgctgctctccagctaggatccaaacgtcgacctgccccgcaggacaagttcgtcatctgcttggtgccagcgtacactgagggcgaggattcattgcgaaaagggcttgattcattgacagctcttcagtatgataacaagaggaaactcatttatgtcatctgcgacggtatgattgtcggtggcggtaatgaccgcccaacgcctaagattgttctggacattctgggagtggatcccaaaattgaccctcctgcattaccgttcaagtcaatcggacaaggtagtgatcagctcaactacggaaaggtctactccggactttatgaatacgaaggcaatgttgtgccctacatcgttgtcgtgaaggtcggcaaagagtcagagcagagcaagtccaagcctggaaacaggggtaagcgtgactctcagattcaaattatgaacttcctcaatcgtgtacatcatcgcgccccaatgtcgcctcttgagctggagattttccaccagatcaacaatgtgattggtgttgaccctgagctctatgaatactgcctgatggtggatgcggatacaagtgtccgagaagattcactcaatcgcctggtcgctgcctgtgctaatgacgctcgtattgccggtatctgtggtgagacaagtttgcaaaatgaggaacgaagctggtggaccatgatccaggtttatgagtactacatttctcatcatctctcaaaagcattcgaatctctctttggcagtgtaacttgtcttcctggatggtaagtacaacacaacattgcaatatacatcacgcagatgctgactcgtacagtttctgtatgtatcgcttgaggacggcggacaaaggccggcccttgatcatatcagacaaggttatcaaagaatatgcagacaacgacgtggacacgctgcacaagaaaaatctgctttctttgggtgaggatcgttacttgactacattgatgacgaagcacttccctaccatgtcctacaaattcatcccggatgcctacgctagcaccgccgcccccgagacgttctccgtcctgctgtctcagcgacgtcgctggatcaactccactgtccataacttggtggaactcgctgctctgaaagacctctgcggcttctgctgcttcagtatgcgctttgtcgtattggtcgatcttcttggaactatcatcctcccagctacctgcgtctacttgggttacctaatctacagtgttgccagtggtgggccaattccaatcatatctatcgccatcttggctggtgtgtacggcctccaggcgattatctttattgtgaagcggcagtggcagcatattggttggatgatcatttatatctgtgcctatccgatctatagtttcgttctgccgatgtattccttctggaaacaggacgacttcagctggggtaacactcgtgttgttcttggagagaagggaaataagcgagttgttgcagtagaagatgaaccattcgaccctcgcagtattcctctccagcgctgggacgattacgctcttgccaataatctgcctggccgccgtggagattataacatgagccaggagaaattctacggaggtcaatatggagatatgggcatggagatggatgatatgcattcccagtattcctcggtcaagcctgcatccacaatcttaaccggatttccaggagcaggccggaatggtagtccttacatgccgccgcagtcgcccgccccgttcggtggaaataccccaggcaacaggcattcgcacctgtccagctttagtcggtataccgatatgccgcttcagccagggcaccagtctcgaaacctatcggtgggaaatctcagccaattccaggatccgtcgaaccggcatagcgtgggactcatgcagagcactgacaatctcctgggggttccccggccaaactctcggagccctgtaggcggttatacctcccggcctcaaagtgcgtttgacttccgcggaagtggtgggcctgatgaaatggccatcacggatgctattcgtagctgcctggccgaagtggacttggacactgtaacgaagaagcaaggtgagcaaatgaatacagccgtgtgatgagatacatgctaactaaactaaccacagttcgcgcattggtcgagcagcggcttcaagcgacattgaccggagacaagcgagcattcctcgatcgccagattgaccaggaattggcaaatatgtaaatttgtagagtgttttctcttccttgtttgttcttgcacccggtaccttaatacatatattcgtgcgggaaaatgttgatatttcggcttggcatgcatggcgtttgggatgatctgaagcattcagcgaatttgaacgagtgtaatagttgtttatgagagttggaatgtactctgggtgttcacctgtacatggagcatactaatgttgtcggacagcatctaatgatgaatttgcaacatgcatgtgttatacatggtagattatagagatctggaatattcaacgcccattca

SEQ ID NO: 32 - protein, 1760 aa, Aspergillus orzae
MSNRYSVYSSHSAAFSTGGRAPQAGGQVSTTTLLNALHAHYTTGQPYQLDAGTSLVVNTLLTATQSSPEGHTGPTIDHELAVRAWEHARRRAEDGCIVLCSAHHSAPSILEPFLAALPLSTPSIAFTALAALRPFLTAVTTFNPSYSLYSALSACYNLTLKGDITGLSLALSTSGINVRKGFLDIPSEPGYRAFDVFYYLLTSASTPAEREFLDLRDASSYALLRKSGTYTPPSYLPTADDAAAAEDFRSALKAIGIKGASQRGLLSVLAGLLKLGNAAGFLVDQEDLEEACEDVGGLLGIDPEVLLHKCSTDDREVLISGIYEALVDWVIGKANEAIASQLQASLDDSSRGSGQAAQWTDDDTVSITVVDLPRPALGKAVAMRGIFDDTLGINAEMKDDGVVVPPAGPAVLNDMTAAIAQVEVDLGITTGPTWREREYELDKKHEVLEKVGLEVEMDSFLRQILFTAESEGITLGKKGRFDLATTLGSSRVWHHISIHPTDDLPENLSPGVPTAAWSAGAVSRQLREWRLAEWANRRLKQIDFTADFDIEEFIGRYFRLGCGEGKDGVENWLVERGWINGDAVVGHQRIWVRENAWWEAETMLDLKPEEPPAASPFMYGGGMLDPGVPHYAVPPIAESTSLLGSRDNLLNRQSTLVPSVAGGAKSIAPSAPHTLHTGGDYGLGTKGDDKKYDSHPYYDDEGRYLGELDPEYADPKHIEKKEITLGRRIWTGFVWALTFWIPSFVLRFVGRMKRPDVRMAWREKLVLVFLILLFNAIVCFYIIAFGNLLCPNKDKVWNEKEVSYHQGNNDFYVSIHGKVYDISKFWKIQHSDTSIETTTSNMEPFMGENLDAYFPPPLTRLCGDFVTDESITLRNNDTNAVLYSNAKHSCGPLQQTDPNTALHKITWYEDVFLPKIDEYYKGSLVWKRSEVSKQADSSSRYWVIKDESIYDLTDYFYTLKQMNNIDSYNFLPSSITELFKNYPGTDVTDKWPNSENATKAQTCLDYVFYKGKVDFRDSARCQVNNYILLAFTCLICAVILVKFLAALQLGSKRRPAPQDKFVICLVPAYTEGEDSLRKGLDSLTALQYDNKRKLIYVICDGMIVGGGNDRPTPKIVLDILGVDPKIDPPALPFKSIGQGSDQLNYGKVYSGLYEYEGNVVPYIVVVKVGKESEQSKSKPGNRGKRDSQIQIMNFLNRVHHRAPMSPLELEIFHQINNVIGVDPELYEYCLMVDADTSVREDSLNRLVAACANDARIAGICGETSLQNEERSWWTMIQVYEYYISHHLSKAFESLFGSVTCLPGCFCMYRLRTADKGRPLIISDKVIKEYADNDVDTLHKKNLLSLGEDRYLTTLMTKHFPTMSYKFIPDAYASTAAPETFSVLLSQRRRWINSTVHNLVELAALKDLCGFCCFSMRFVVLVDLLGTIILPATCVYLGYLIYSVASGGPIPIISIAILAGVYGLQAIIFIVKRQWQHIGWMIIYICAYPIYSFVLPMYSFWKQDDFSWGNTRVVLGEKGNKRVVAVEDEPFDPRSIPLQRWDDYALANNLPGRRGDYNMSQEKFYGGQYGDMGMEMDDMHSQYSSVKPASTILTGFPGAGRNGSPYMPPQSPAPFGGNTPGNRHSHLSSFSRYTDMPLQPGHQSRNLSVGNLSQFQDPSNRHSVGLMQSTDNLLGVPRPNSRSPVGGYTSRPQSAFDFRGSGGPDEMAITDAIRSCLAEVDLDTVTKKQVRALVEQRLQATLTGDKRAFLDRQIDQELANM

SEQ ID NO: 33 - DNA, 6393 bp, Aspergillus orzae
cctgactggattcgaatatcctcccagagtaataatcataatcaccgcggtttcaaccatcgttccatccatctgcaagtcattctccagatacgatcgctacaattctcttccacgccactctcgctttgaaattcgccagctgttcatctcgcattcactgcaccaaatttccgcagaaaatcaacgagccgcgtgctgtttgaggacccgtttgccgtctaagcaatgtgcctatcttttctctattgataagacatattctccattccagggcgttcccacattccaagccatccagcacctcatctaaatcagcataactgaacggccttatccagttcgagaacacgccaggcaagatcgctttctgggctttttccctccgtatatccttcaccggcatccttaccgatcttccgtaagacgcccataccgatcgtaactattgtgaccggtgcttaagaaaccccagcgtcgaggtttcgagccattgataatatggtcgggccttcgccagctgggacagttccgtcccatgcgcagtcgtcgctaccctcgttaccagcccatctgcaatcagacacgcatttgactgcccatttagccagccggtatgattctgcgatctactgtatctccatttataactgacatcgtgggtgttcttctaggtttcatgtcggcttaccaacagctcgcctgtcttctcaggctctgatcagtctcaacacatacacttcatcttcaaagggccctgatggcggcaaggagggcagtgctatgggcgaggcggaagacctcgcccgtcgtgccttcactcgcctaggtgcccgtggagagaatcaggctattgtctttctgtaagcgcaatttggacatatacacacctacatgctctagattctaattgacgatttgcagcggggagagcggggctggcaaaactacccttcgagcgcatgtgttgtcgtcctttctctcgttctcgtcgacgccattgtcatccaaattgtcgtacgcctcctttatttttgacacattgacaacgaccaagtctttgaccacactcacggcatcgaaggcggggcttttcttggaactacagtatgacggttcgtcatcggtcaacccaacgctgatcggtggtaaaattatcgaccacagactcgaacgcagtcgtattgcgtcagtccccacaggtgaaagaagcttccatgtcctttattacctcttagctgggacaagtgcggcggaaaagagtcacttgggcttcgataactctatccatgtttcgacgaatgccggcaagctttcgtccgcatcaataggtcacaagaggtggagatatctgggccatccaactcagctaaaggtgggcatcaatgatgcggaggggttccaacatttcaagacagcgctcaggaaactcgagtttccacgcagtgagatagccgagatttgccaaattctcgctgtgatacttcatatcggccaactagacttcgccagcggacaagctactttgacatcagcggaggagtctggcggctattcccacgagggtggggagactgtcacggtagtgaagaataaggatgtcctatcaagcgttgccgcattcttgggtcttggagttgatgaacttgagaatagttttagctaccggaccaagaccatccaccgggagcgcgtcacggtgatgctggacccaaagggtgcccggcaaaatgctgatgaacttgcgcgcaccatttactccctactagtggcttatattctcgagaatgtcaatcagagaatatgcgcggcggaggatagcgttgccaacaccgtttctattgttgatttccctggtttctcccaagcatgctcaaccggatccacactggatcaacttcttagcaatgcagccaccgaatcgttgtacaatttctgcctacagtccttcttcgaccgcaaggctgatatgttagagcgcgaggaggttgtggtccctgcaacaagctactttgacaacactgatgctgtccgtgggctgttgaagcatggcaatgggcttctcagcatcctcgatgatcaaaccaggcgcggtagaacggaagctcaatttgccgagtcgttgaagaaacggttcgaaaacaaaaacccagctattgttgtcggaagctcaggatctactcacggaactggatatgtctcgcagcaagcccgctcggcatttactgtgaaacattttgctggtgaagttgattattccatctccggtctattggaggaaaatggagaagttatctccggtgacctgatgaacttgatgaagtccacccggagcgacttcgtgagagagctctttggtcaagaagcactacagacagtgacccacccaaaggagaagaccgctattatgcaagctcaggtaagctcaaagcccttgagaatgcctagcatggcaaggcgaaaagccagcccagcatctcgtcttactttcgatgcacctacggcggaagaacccgaagacaatgagagctatgggggtagtacagccaagagctccggaaggcggaagagcgcgatgtctatgaccggcatgcagggtgctgccggacaattcctgtcctcgcttgagatcgtcaataagtgtctcagctcccctagtttgaatccatatttcatcttttgtttgaagccaaacgaccggcgaatcgcgaatcaatttgacagcaaatgtgtgcgagcccaggtgcaaacatttggtattgctgagatcagccaacgtctgaggaacgcggatttcagcgttttccttccctttgaggaattccttggtttggccgaagtaggcaacgtcgtggtgggaagtgataaagaaaagtcagaggttgtgctagatgagaagcggtggccgggtaatgaagctcgtgttggcagcacgggtgttttcctgagcgaacgctgctgggcagacctcgcaaaggtgggcgagcgtgttgttcctgtctaccatgccgacggctcggacgaaggtggtgatggtctccttcatccacgtactgctgggtatggggattccaaggttcgtcttctcaacccggcggaccagtctctgggcaactttatatatggcgatgaaagcaagcaaggatatttcgggagtcgcgatattgatgggcgatctgacactggtggctctggcctgaactcgggtgatatgttccacaatctcgagacaagagagcagatgttggaaaaagggaatgagaagaagatggaagaggtggatgaagtacctgtttctggcagtcgcaaacgctggatggcaatcgtctggttgctcacattctacattccggactttgccatcaggctcttcggtcggatgaaacgcaaggacgtacgaacggcttggcgtgagaaatttgcgatcaatttgattatttggttcagttgtgccgtcgccattttctttatcgtcgctttccctggtttggtatgtccgacacagcatgtgtattcagccgcggaattggaatctcataacggcaaaaatggccatgattcttatattgccattcgcggcgtggtttttgaccttgataaattcatgccccgacactatcccgacattgtgccgcaatcttctttgaagaaatatgccggcatggacgctaccggtctctttcctgtgcaagtgtcagcgttatgccaaggtaaggatgggtcgatcgatcccactgttcttctggattacacccctacgaacatctccggatcggccacaaccatcagcacgggagatctcaacgcaaaataccatgatttccgctactacaccaacgactcccgtccagactggttcgcagagcagatgaaagaactgagggcgacttatctgaaaggatacattggttatacgccacaatacatctccacgttagccaaaaagtcccagaacattggaagcatagacggaaaagtctacgatttgacaacttatatcagtggaggtcgacgggtcgcagctcctaccggaaaagaggtcccggctaatgtcgaccgagagttcatggatcctttggttgtgtcgctctttcaagatcttcccggacaggatttgagtaaacactgggaacagctgcagatagatgcaggcatgcgtgatcggatgcagatgtgtctggataaccttttcttcgtcggcaaggttgatacgcgtaattcagctcaatgtcaattcgcgcgttacttcatccttgcaatctcaatcttgatttgtgctgttgttatcttcaaatttgctgccgctttacaatttggcaagaaaaatgtcccggagaatcttgataaattcatcatttgtcaggtgccggcatacactgaagatgaggagtcccttcgtcgtgcaatggattctatggcccgcatgcagtatgacgacaagcgcaaactccttgttgtcatctgtgatggtatgattattggacaaggaaacgatcgacctactccacggattgtattagatatcctgggcgtcccagaatcagtggatcccgagcccctcagctttgagagtttgggcgaaggcatgaagcagcacaacatgggtaaaatttattctggtctctatgaggtacagggacatatcgtacctttccttgttgtcgttaaagtcggaaagccttctgaagtctcccgacccggtaaccgtggaaaacgtgactcccagatggtactcatgcgctttttgaaccgcgtgcattacaaccttcccatgagtcccatggaactggaaatgtatcaccaaattcgtaacatcattggcgtcaatcccacgttctacgagttcattctgcaagtcgacgctgatacggtggttgcacccgattccggaactcgattcgtcgcttcttgccttgccgacacacgcattatcggtatctgtggcgaaacaggcttgacaaatgctaagcattcagcggtgaccatgatccaagtttacgagtacttcatctcccacaatctgatcaaggctttcgaaagtcttttcgggtcagtcacatgtttgccgggctgcttcaccatgtaccgtattcgttctgcagaaaccgcaaaaccgcttttcgttagcaaggaggttgtggaagcctactcggaaattcgcgtcgacacactccatatgaagaacctgctacatctgggtgaggatcggtacttaacaacccttctcttgaagcatcatcctagtttcaagacgaagttcctatttgcagctaaggcctggacgatcgcacctgaaagcttctcagtcttcttatcgcaacgtcgtagatggatcaactcaactgtacacaatctgattgaactgatccctcttcagcaactttgtggtttctgttgctttagtatgagattcatcgtcttcgttgaccttctcagtacctgtatccaacctgtttcgctcgcctatatcatctacttgattgtctggcttgccagagattcgtccaccattccatggacgtcttttgttctcatcgcagcgatctatggtcttcaggccctcatcttcatcttccgccgcaagtgggaaatgattggctggatgattgtgtatctcctggccatgcctatattctccgtggccttgcccttctactcgttctggcacatggatgacttctcttggggaaacacccgcgtcatcactggagaaaagggccgcaaagtcgtcatttcagatgaaggaaagtttgaccccgcctctatcccgaaaaagagatgggaggagtatcaagcggagctctgggaggcccagacgtcgcgagacgaccgctcagaagtttcaggcttctcatatggtaccaagtcgtatcaccctgcgcagtccgaatacgggttccctggagctagaccaatgtcgcagttcgatcttcctcgctatgggtccaggatgtctctagctccttccgagatgatgagccgtcatatggacatggaaatggaggatctctcacatctgcctagcgacgatgccattctcgcggaaatccgtgaaatcctgcggacagccgacctgatgaccgtaacgaagaagagtatcaaacaagagctagagaggcgttttggtgtgaatctcgatgccaagcgtccatatatcaactcaggtaagagatgttccccgttaagatattaccacagagtcttactaactgtttgtgacccttatagccaccgaagctgttctatcgggcgcgctctaatctgagggtcaagctcgatgtgtattgcatggaagaccgttaccttttccacggtgtataaaaatttcttatctgatttaatattcgcatatccattgtcgcaacttataaactcgcagctgtgattgtttgt

SEQ ID NO: 34 - protein, 1857 aa, Aspergillus orzae
MVGPSPAGTVPSHAQSSLPSLPAHLQSDTHLTAHLASRFHVGLPTARLSSQALISLNTYTSSSKGPDGGKEGSAMGEAEDLARRAFTRLGARGENQAIVFLGESGAGKTTLRAHVLSSFLSFSSTPLSSKLSYASFIFDTLTTTKSLTTLTASKAGLFLELQYDGSSSVNPTLIGGKIIDHRLERSRIASVPTGERSFHVLYYLLAGTSAAEKSHLGFDNSIHVSTNAGKLSSASIGHKRWRYLGHPTQLKVGINDAEGFQHFKTALRKLEFPRSEIAEICQILAVILHIGQLDFASGQATLTSAEESGGYSHEGGETVTVVKNKDVLSSVAAFLGLGVDELENSFSYRTKTIHRERVTVMLDPKGARQNADELARTIYSLLVAYILENVNQRICAAEDSVANTVSIVDFPGFSQACSTGSTLDQLLSNAATESLYNFCLQSFFDRKADMLEREEVVVPATSYFDNTDAVRGLLKHGNGLLSILDDQTRRGRTEAQFAESLKKRFENKNPAIVVGSSGSTHGTGYVSQQARSAFTVKHFAGEVDYSISGLLEENGEVISGDLMNLMKSTRSDFVRELFGQEALQTVTHPKEKTAIMQAQVSSKPLRMPSMARRKASPASRLTFDAPTAEEPEDNESYGGSTAKSSGRRKSAMSMTGMQGAAGQFLSSLEIVNKCLSSPSLNPYFIFCLKPNDRRIANQFDSKCVRAQVQTFGIAEISQRLRNADFSVFLPFEEFLGLAEVGNVVVGSDKEKSEVVLDEKRWPGNEARVGSTGVFLSERCWADLAKVGERVVPVYHADGSDEGGDGLLHPRTAGYGDSKVRLLNPADQSLGNFIYGDESKQGYFGSRDIDGRSDTGGSGLNSGDMFHNLETREQMLEKGNEKKMEEVDEVPVSGSRKRWMAIVWLLTFYIPDFAIRLFGRMKRKDVRTAWREKFAINLIIWFSCAVAIFFIVAFPGLVCPTQHVYSAAELESHNGKNGHDSYIAIRGVVFDLDKFMPRHYPDIVPQSSLKKYAGMDATGLFPVQVSALCQGKDGSIDPTVLLDYTPTNISGSATTISTGDLNAKYHDFRYYTNDSRPDWFAEQMKELRATYLKGYIGYTPQYISTLAKKSQNIGSIDGKVYDLTTYISGGRRVAAPTGKEVPANVDREFMDPLVVSLFQDLPGQDLSKHWEQLQIDAGMRDRMQMCLDNLFFVGKVDTRNSAQCQFARYFILAISILICAVVIFKFAAALQFGKKNVPENLDKFIICQVPAYTEDEESLRRAMDSMARMQYDDKRKLLVVICDGMIIGQGNDRPTPRIVLDILGVPESVDPEPLSFESLGEGMKQHNMGKIYSGLYEVQGHIVPFLVVVKVGKPSEVSRPGNRGKRDSQMVLMRFLNRVHYNLPMSPMELEMYHQIRNIIGVNPTFYEFILQVDADTVVAPDSGTRFVASCLADTRIIGICGETGLTNAKHSAVTMIQVYEYFISHNLIKAFESLFGSVTCLPGCFTMYRIRSAETAKPLFVSKEVVEAYSEIRVDTLHMKNLLHLGEDRYLTTLLLKHHPSFKTKFLFAAKAWTIAPESFSVFLSQRRRWINSTVHNLIELIPLQQLCGFCCFSMRFIVFVDLLSTCIQPVSLAYIIYLIVWLARDSSTIPWTSFVLIAAIYGLQALIFIFRRKWEMIGWMIVYLLAMPIFSVALPFYSFWHMDDFSWGNTRVITGEKGRKVVISDEGKFDPASIPKKRWEEYQAELWEAQTSRDDRSEVSGFSYGTKSYHPAQSEYGFPGARPMSQFDLPRYGSRMSLAPSEMMSRHMDMEMEDLSHLPSDDAILAEIREILRTADLMTVTKKSIKQELERRFGVNLDAKRPYINSATEAVLSGAL

SEQ ID NO: 35 - DNA, 906 bp, Saccharomyces cerevisiae
  CDS (1)...(906)
atgaaaattttcaatacaatacaatctgtgctgttcgcagcattttttctaaaacagggaaattgccttgcgtcaaatgggagtaccgcattgatgggggaagtagatatgcaaacgccctttccagagtggttaacagaatttactaatcttacacaatggcctggaattgacccaccttatattccgctagattacataaatcttactgaagtgccagaattagataggtactatcctggccagtgtcccaaaatttctagagagcagtgctcatttgactgctataactgcatcgatgttgatgatgtaacttcgtgtttcaaactttcccaaacatttgacgacggtccggccccggcgacagaggcattgctcaagaaattgagacaaagaaccactttttttgttctggggataaacactgttaattatcctgatatatatgagcatattttagagaggggtcatttgattggtacacacacgtggtcacatgaattcttgccaagtttatcaaacgaagaaattgtagcccaaattgaatggtcaatttgggctatgaatgccacaggcaaacatttccccaagtattttaggcctccatacggtgcaattgataatagggttagagctatagtaaaacagtttggcctaacggttgtcttgtgggatctcgatacttttgattggaaattaatcactaatgatgatttcagaacagaggaagaaatacttatggacataaatacttggaagggaaaacggaaaggtttgatcttagagcacgatggtgcacgaagaacagttgaggttgctattaaaatcaacgaacttattggtagtgaccaattgacaattgcagaatgtattggtgatacagactacatcgaacgctacgactag

SEQ ID NO: 36 - protein, 301 aa, Saccharomyces cerevisiae
MKIFNTIQSVLFAAFFLKQGNCLASNGSTALMGEVDMQTPFPEWLTEFTNLTQWPGIDPPYIPLDYINLTEVPELDRYYPGQCPKISREQCSFDCYNCIDVDDVTSCFKLSQTFDDGPAPATEALLKKLRQRTTFFVLGINTVNYPDIYEHILERGHLIGTHTWSHEFLPSLSNEEIVAQIEWSIWAMNATGKHFPKYFRPPYGAIDNRVRAIVKQFGLTVVLWDLDTFDWKLITNDDFRTEEEILMDINTWKGKRKGLILEHDGARRTVEVAIKINELIGSDQLTIAECIGDTDYIERYD

SEQ ID NO: 37 - DNA, 939 bp, Saccharomyces cerevisiae
  CDS (1)...(939)
atgagaatacaactaaatacaattgatttgcaatgtattattgcactttcctgtctggggcaatttgttcacgcggaagctaatagggaagatttaaagcagatagactttcaatttcctgtattggaaagggcagctacaaaaacgccttttccggattggcttagtgcatttaccgggttaaaagaatggcctgggttagatccaccttatatacctttagatttcattgatttcagtcaaattccagattataaggaatatgatcaaaaccattgcgacagtgttccaagggactcgtgctctttcgattgccatcactgcaccgaacacgatgatgtgtacacatgttccaaactttcccagacatttgacgatggtccttctgcttccactactaaattattggaccggttgaagcataattccaccttcttcaatttaggtgtcaatatagttcaacatccagatatctatcaaagaatgcaaaaggagggacacttaatcggctcacatacctggtctcacgtatatttgccaaatgtatcgaatgaaaaaattatagctcaaattgaatggtccatctgggcgatgaatgctactggcaaccatacccccaaatggttcagacctccatatggcggaatagataatagagtaagagcaataacaaggcaatttggcttacaagccgtcttatgggatcacgatacttttgattggagcctccttctcaatgattctgtcataactgaacaagaaattcttcaaaatgtaataaactggaacaagtcaggaaccggattaatattagaacacgattcaacggaaaaaactgtcgatcttgccattaaaataaataagttgataggtgatgatcaatcaacagtttctcattgtgtcggcggaattgattacataaaagaattcttgtcctaa

SEQ ID NO: 38 - protein, 312 aa, Saccharomyces cerevisiae
MRIQLNTIDLQCIIALSCLGQFVHAEANREDLKQIDFQFPVLERAATKTPFPDWLSAFTGLKEWPGLDPPYIPLDFIDFSQIPDYKEYDQNHCDSVPRDSCSFDCHHCTEHDDVYTCSKLSQTFDDGPSASTTKLLDRLKHNSTFFNLGVNIVQHPDIYQRMQKEGHLIGSHTWSHVYLPNVSNEKIIAQIEWSIWAMNATGNHTPKWFRPPYGGIDNRVRAITRQFGLQAVLWDHDTFDWSLLLNDSVITEQEILQNVINWNKSGTGLILEHDSTEKTVDLAIKINKLIGDDQSTVSHCVGGIDYIKEFLS

SEQ ID NO: 39 - DNA, 1375 bp, Mucor rouxii
tctacatccaaaaagaattgaaacgatgcaaatcaagacattcgccctttcagctgcaattgcacaagttgctactcttgctttagccgacacctccgcaaattactggcaatcatttacttctcaaattaatcccaagaacatctccattccctctattgagcaaacttcatccattgaccccactcaagaatgtgcttactacactcctgatgcttcattgttcacattcaacgcttccgaatggccctctatctgggaagtcgctactaccaatggtatgaatgagtctgccgagttcctcagtgtctacaattctattgactggaccaaggcacccaatatttctgtgcgtacccttgacgctaacggcaacttggataccactggttacaatactgctactgaccctgattgttggtggacagctaccacatgtacctctcccaagatttctgatatcaatgacgatatctccaagtgtcctgaacccgagacttggggtttgacttacgatgatggacctaactgctctcacaacgctttctatgactaccttcaagagcaaaagttgaaggcctccatgttttatattggttccaatgttgttgactggccatacggtgctatgcgtggtgttgttgatggccatcacattgcatcccacacatggtctcaccctcaaatgacaaccaagaccaatcaagaggtccttgctgaattctattatactcaaaaggccatcaagctcgctactggtttgacccctcgttactggcgtcctccttatggtgatatcgatgatcgtgttcgttggattgcctctcaattaggtttaactgctgttatttggaacctcgatactgatgattggtctgctggtgtcactactactgtcgaagctgttgagcaaagttattccgattatattgctatgggtaccaatggtacttttgccaacagtggtaacattgtattgacccatgaaatcaacacaactatgagtctcgctgtcgagaacttgcccaagatcatttctgcctataaacaagtcatcgatgtcgctacctgttacaacatttctcacccttactttgaagactacgaatggaccaatgtcttgaacggcacaaaatcttctgctaccgccagtggatctgctacttctgctagtgcttctggaggcgctactaccgctgccgctcatatccaagcttctactagcggcgccatgtctgtccttcccaacctcgccttgatctctgccttcattgctaccctgttgttttagtcaaacatcgtcatatatcaccttcctgtcataatttataatagtaaaacatcatatttagatttttctacatcttaaaaaaaa

SEQ ID NO: 40 - protein, 421 aa, Mucor rouxii
MQIKTFALSAAIAQVATLALADTSANYWQSFTSQINPKNISIPSIEQTSSIDPTQECAYYTPDASLFTFNASEWPSIWEVATTNGMNESAEFLSVYNSIDWTKAPNISVRTLDANGNLDTTGYNTATDPDCWWTATTCTSPKISDINDDISKCPEPETWGLTYDDGPNCSHNAFYDYLQEQKLKASMFYIGSNVVDWPYGAMRGVVDGHHIASHTWSHPQMTTKTNQEVLAEFYYTQKAIKLATGLTPRYWRPPYGDIDDRVRWIASQLGLTAVIWNLDTDDWSAGVTTTVEAVEQSYSDYIAMGTNGTFANSGNIVLTHEINTTMSLAVENLPKIISAYKQVIDVATCYNISHPYFEDYEWTNVLNGTKSSATASGSATSASASGGATTAAAHIQASTSGAMSVLPNLALISAFIATLLF

SEQ ID NO: 41 - DNA, 1293 bp, Gongronella butleri
  CDS (1)...(1293)
atgcgccgccttggacagggcatccaattggtgtccgctgactattggtccaacttcaattcgagcgtgaaccctctcaacgtgaaggtgccccaaatcacacagccacgtcgattgatcacgtcacggagtgcacggtactacacgcctgatccactcgttgatcaccatcacgcccaccggctggggcccaattggctcacggccaccaccaatggcatgaacaccagtgctgaattcaccgccctttacaactcgatcaattgggacaacggcggcccaaacatctcggtgcgcaccttcaacaccgatggctccatgaacactaatggctacgacgtggccaatgatcccgattgttggtggactgtctctggctgcacggtgcccaagctccaggatgtcaatgctgacatttacaagtgtcctgagcccgatacgtggggcttgttctatgacgacggccccaattgctcgcacaatgccttttacaactttttgcaggagcaaaatctgcgcgcttccatgttttacattggttccaacgtcatgaactggccctatggtgccatgcgtggtgtccaagatggccatcacattgctttccacacctggtcccatcagtcattgacgaccctgacgaaccaagaagggctcgccgagttctactacacgcaaaagatgattcacttggccactggtgtgacacctcgctactggcgcgctccctatggtgatgtcgatgatcgtgtgcgctggattgccacgcaattgaacctgacgacgatcctctgggactatgataccaacgactggcaggcaggcgacggtgtgcccgagtccacggtgcaaaacacgtacaatgaattcatccagatgggcaacaatggttcgatggccagcggtggcaacattgtactgacgcacgagatcaacaacacgacgatgcaattggccgtcgagaacatccccaacatgctcaagtcttacaagcacgtcgtcaacgttgccacctgcatgaacatcacgttcccgacatggagcagaccggtgcctttcccttctttagccaatttattgcgcaaaacagcttgggtgcgggcggtgccgccttcaacattaccacgggcgccggggctcagagctcaagctcgttttctcgttgggcgccatgtctttgcccaagccaatgccggtgtcgccgttttgatgacagtccgtgggccgttgcgctttgcatttaaatcttttccaatgggacccccttttctcccttccttatatgttttttga

SEQ ID NO: 42 - protein, 430 aa, Gongronella butleri
MRRLGQGIQLVSADYWSNFNSSVNPLNVKVPQITQPRRLITSRSARYYTPDPLVDHHHAHRLGPNWLTATTNGMNTSAEFTALYNSINWDNGGPNISVRTFNTDGSMNTNGYDVANDPDCWWTVSGCTVPKLQDVNADIYKCPEPDTWGLFYDDGPNCSHNAFYNFLQEQNLRASMFYIGSNVMNWPYGAMRGVQDGHHIAFHTWSHQSLTTLTNQEGLAEFYYTQKMIHLATGVTPRYWRAPYGDVDDRVRWIATQLNLTTILWDYDTNDWQAGDGVPESTVQNTYNEFIQMGNNGSMASGGNIVLTHEINNTTMQLAVENIPNMLKSYKHVVNVATCMNITFPTWSRPVPFPSLANLLRKTAWVRAVPPSTLPRAPGLRAQARFLVGRHVFAQANAGVAVLMTVRGPLRFAFKSFPMGPPFLPSLYVF

SEQ ID NO: 43 - DNA, 631 bp, Paramecium bursaria Chlorella virus CVK2
  CDS (1)...(631)
atgctgcctctcaaaatcaagagtagcatactgtatgcggttatcttggccattaactttggattgctttcgctcgtcattatatttcatgagtattggtatgcatttgcgcctattcttgtactcggcgctgcgtcttctctgtggtatattgcgtgggtgcttatgcatcgtgtatacttaggtttcaaaggaaaacccgtgctgaccgcccccaaagaacctatgatgttcctcgtcacagcgtatcgcgagacgaaggaagaacttgatagaaccgtggagtccgttacgatgcaaaaaatagaccccgaggttagcaagactgttgttgttattgttgatggtgagaaggaaactgcacacgaactacgaaagtataaccagtatgatgaaactttcgtcatcaaagatgcatatgaggattggcataataagccaaaggatgttacaattttcaagaaaatacataatggtattgacgtcgtatatctcataaaaagtgagaacgcgggaaaacgtgatagcgttgtgcttgcacgaactcttgcatacggaaatctgttcgaacatagtgaaaacagacatgctatgaaaatttcaggcgaattagacctcatatggtctcgtt

SEQ ID NO: 44 - protein, 210 aa, Paramecium bursaria Chlorella virus CVK2
MLPLKIKSSILYAVILAINFGLLSLVIIFHEYWYAFAPILVLGAASSLWYIAWVLMHRVYLGFKGKPVLTAPKEPMMFLVTAYRETKEELDRTVESVTMQKIDPEVSKTVVVIVDGEKETAHELRKYNQYDETFVIKDAYEDWHNKPKDVTIFKKIHNGIDVVYLIKSENAGKRDSVVLARTLAYGNLFEHSENRHAMKISGELDLIWSR